SEQUENCE LISTING (62 sequences)

SEQ ID NO: 1 - protein, 536 aa, Artificial sequence
  Synthetic polypeptide
MALRNVPFRSEVLGWDPDSLADYFKKLNYKDCEKAVKKYHIDGARFLNLTENDIQKFPKLRVPILSKLSQEINKNEERRSIFTRKPQVPRFPEETESHEEDNGGWSSFEEDDYESPNDDQDGEDDGDYESPNEEEEAPVEDDADYEPPPSNDEEALQNSILPAKPFPNSNSMYIDRPPSGKTPQQPPVPPQRPMAALPPPPAGRNHSPLPPPQTNHEEPSRSRNHKTAKLPAPSIDRSTKPPLDRSLAPFDREPFTLGKKPPFSDKPSIPAGRSLGEHLPKIQKPPLPPTTERHERSSPLPGKKPPVPKHGWGPDRRENDEDDVHQRPLPQPALLPMSSNTFPSRSTKPSPMNPLPSSHMPGAFSESNSSFPQSASLPPYFSQGPSNRPPIRAEGRNFPLPLPNKPRPPSPAEEENSLNEEWYVSYITRPEAEAALRKINQDGTFLVRDSSKKTTTNPYVLMVLYKDKVYNIQIRYQKESQVYLLGTGLRGKEDFLSVSDIIDYFRKMPLLLIDGKNRGSRYQCTLTHAAGYPGNS

SEQ ID NO: 2 - protein, 262 aa, Artificial sequence
  Synthetic polypeptide
MEEAILVPCVLGLLLLPILAMLMALCVHCHRLPGSYDSTSSDSLYPRGIQFKRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 3 - protein, 509 aa, Artificial sequence
  Synthetic polypeptide
MGCGCSSHPEDDWMENIDVCENCHYPIVPLDGKGTLLIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQYQPQP

SEQ ID NO: 4 - protein, 300 aa, Artificial sequence
  Synthetic polypeptide
RKRRGSGATNFSLLKQAGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 5 - protein, 301 aa, Artificial sequence
  Synthetic polypeptide
RRKRRGSGATNFSLLKQAGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 6 - protein, 18 aa, Artificial sequence
  Synthetic polypeptide
YPYDVPDYAYPYDVPDYA

SEQ ID NO: 7 - protein, 836 aa, Artificial sequence
  Synthetic polypeptide
MALRNVPFRSEVLGWDPDSLADYFKKLNYKDCEKAVKKYHIDGARFLNLTENDIQKFPKLRVPILSKLSQEINKNEERRSIFTRKPQVPRFPEETESHEEDNGGWSSFEEDDYESPNDDQDGEDDGDYESPNEEEEAPVEDDADYEPPPSNDEEALQNSILPAKPFPNSNSMYIDRPPSGKTPQQPPVPPQRPMAALPPPPAGRNHSPLPPPQTNHEEPSRSRNHKTAKLPAPSIDRSTKPPLDRSLAPFDREPFTLGKKPPFSDKPSIPAGRSLGEHLPKIQKPPLPPTTERHERSSPLPGKKPPVPKHGWGPDRRENDEDDVHQRPLPQPALLPMSSNTFPSRSTKPSPMNPLPSSHMPGAFSESNSSFPQSASLPPYFSQGPSNRPPIRAEGRNFPLPLPNKPRPPSPAEEENSLNEEWYVSYITRPEAEAALRKINQDGTFLVRDSSKKTTTNPYVLMVLYKDKVYNIQIRYQKESQVYLLGTGLRGKEDFLSVSDIIDYFRKMPLLLIDGKNRGSRYQCTLTHAAGYPGNSRKRRGSGATNFSLLKQAGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 8 - protein, 563 aa, Artificial sequence
  Synthetic polypeptide
MEEAILVPCVLGLLLLPILAMLMALCVHCHRLPGSYDSTSSDSLYPRGIQFKRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELNRRKRRGSGATNFSLLKQAGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 9 - protein, 527 aa, Artificial sequence
  Synthetic polypeptide
MGCGCSSHPEDDWMENIDVCENCHYPIVPLDGKGTLLIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQYQPQPYPYDVPDYAYPYDVPDYA

SEQ ID NO: 10 - protein, 32 aa, Artificial sequence
  Synthetic polypeptide
MARSVTLVFLVLVSLTGLYAAYTDIEMNRLGK

SEQ ID NO: 11 - protein, 264 aa, Artificial sequence
  Synthetic polypeptide
MARSVTLVFLVLVSLTGLYAADIQMTQSPSSLSASVGDRVTITCRASQDVNTAVAWYQQKPGKAPKLLIYSASFLESGVPSRFSGSRSGTDFTLTISSLQPEDFATYYCQQHYTTPPTFGQGTKVEIKGSTSGSGKPGSGEGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 12 - protein, 72 aa, Artificial sequence
  Synthetic polypeptide
AATTTPAPRPPTPAPTIASQPLSLRPEACRPAAGGAVHTRGLDFACDIYIWAPLAGTCGVLLLSLVITLYCK

SEQ ID NO: 13 - protein, 68 aa, Artificial sequence
  Synthetic polypeptide
AAIEVMYPPPYLDNEKSNGTIIHVKGKHLCPSPLFPGPSKPFWVLVVVGGVLACYSLLVTVAFIIFWV

SEQ ID NO: 14 - protein, 542 aa, Artificial sequence
  Synthetic polypeptide
GSGGGGSALRNVPFRSEVLGWDPDSLADYFKKLNYKDCEKAVKKYHIDGARFLNLTENDIQKFPKLRVPILSKLSQEINKNEERRSIFTRKPQVPRFPEETESHEEDNGGWSSFEEDDYESPNDDQDGEDDGDYESPNEEEEAPVEDDADYEPPPSNDEEALQNSILPAKPFPNSNSMYIDRPPSGKTPQQPPVPPQRPMAALPPPPAGRNHSPLPPPQTNHEEPSRSRNHKTAKLPAPSIDRSTKPPLDRSLAPFDREPFTLGKKPPFSDKPSIPAGRSLGEHLPKIQKPPLPPTTERHERSSPLPGKKPPVPKHGWGPDRRENDEDDVHQRPLPQPALLPMSSNTFPSRSTKPSPMNPLPSSHMPGAFSESNSSFPQSASLPPYFSQGPSNRPPIRAEGRNFPLPLPNKPRPPSPAEEENSLNEEWYVSYITRPEAEAALRKINQDGTFLVRDSSKKTTTNPYVLMVLYKDKVYNIQIRYQKESQVYLLGTGLRGKEDFLSVSDIIDYFRKMPLLLIDGKNRGSRYQCTLTHAAGYPGNS

SEQ ID NO: 15 - protein, 477 aa, Artificial sequence
  Synthetic polypeptide
GSIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQYQPQPGNS

SEQ ID NO: 16 - protein, 646 aa, Artificial sequence
  Synthetic polypeptide
MARSVTLVFLVLVSLTGLYAAYTDIEMNRLGKAATTTPAPRPPTPAPTIASQPLSLRPEACRPAAGGAVHTRGLDFACDIYIWAPLAGTCGVLLLSLVITLYCKGSGGGGSALRNVPFRSEVLGWDPDSLADYFKKLNYKDCEKAVKKYHIDGARFLNLTENDIQKFPKLRVPILSKLSQEINKNEERRSIFTRKPQVPRFPEETESHEEDNGGWSSFEEDDYESPNDDQDGEDDGDYESPNEEEEAPVEDDADYEPPPSNDEEALQNSILPAKPFPNSNSMYIDRPPSGKTPQQPPVPPQRPMAALPPPPAGRNHSPLPPPQTNHEEPSRSRNHKTAKLPAPSIDRSTKPPLDRSLAPFDREPFTLGKKPPFSDKPSIPAGRSLGEHLPKIQKPPLPPTTERHERSSPLPGKKPPVPKHGWGPDRRENDEDDVHQRPLPQPALLPMSSNTFPSRSTKPSPMNPLPSSHMPGAFSESNSSFPQSASLPPYFSQGPSNRPPIRAEGRNFPLPLPNKPRPPSPAEEENSLNEEWYVSYITRPEAEAALRKINQDGTFLVRDSSKKTTTNPYVLMVLYKDKVYNIQIRYQKESQVYLLGTGLRGKEDFLSVSDIIDYFRKMPLLLIDGKNRGSRYQCTLTHAAGYPGNS

SEQ ID NO: 17 - protein, 581 aa, Artificial sequence
  Synthetic polypeptide
MARSVTLVFLVLVSLTGLYAAYTDIEMNRLGKAATTTPAPRPPTPAPTIASQPLSLRPEACRPAAGGAVHTRGLDFACDIYIWAPLAGTCGVLLLSLVITLYCKGSIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQYQPQPGNS

SEQ ID NO: 18 - protein, 878 aa, Artificial sequence
  Synthetic polypeptide
MARSVTLVFLVLVSLTGLYAADIQMTQSPSSLSASVGDRVTITCRASQDVNTAVAWYQQKPGKAPKLLIYSASFLESGVPSRFSGSRSGTDFTLTISSLQPEDFATYYCQQHYTTPPTFGQGTKVEIKGSTSGSGKPGSGEGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSAATTTPAPRPPTPAPTIASQPLSLRPEACRPAAGGAVHTRGLDFACDIYIWAPLAGTCGVLLLSLVITLYCKGSGGGGSALRNVPFRSEVLGWDPDSLADYFKKLNYKDCEKAVKKYHIDGARFLNLTENDIQKFPKLRVPILSKLSQEINKNEERRSIFTRKPQVPRFPEETESHEEDNGGWSSFEEDDYESPNDDQDGEDDGDYESPNEEEEAPVEDDADYEPPPSNDEEALQNSILPAKPFPNSNSMYIDRPPSGKTPQQPPVPPQRPMAALPPPPAGRNHSPLPPPQTNHEEPSRSRNHKTAKLPAPSIDRSTKPPLDRSLAPFDREPFTLGKKPPFSDKPSIPAGRSLGEHLPKIQKPPLPPTTERHERSSPLPGKKPPVPKHGWGPDRRENDEDDVHQRPLPQPALLPMSSNTFPSRSTKPSPMNPLPSSHMPGAFSESNSSFPQSASLPPYFSQGPSNRPPIRAEGRNFPLPLPNKPRPPSPAEEENSLNEEWYVSYITRPEAEAALRKINQDGTFLVRDSSKKTTTNPYVLMVLYKDKVYNIQIRYQKESQVYLLGTGLRGKEDFLSVSDIIDYFRKMPLLLIDGKNRGSRYQCTLTHAAGYPGNS

SEQ ID NO: 19 - protein, 874 aa, Artificial sequence
  Synthetic polypeptide
MARSVTLVFLVLVSLTGLYAADIQMTQSPSSLSASVGDRVTITCRASQDVNTAVAWYQQKPGKAPKLLIYSASFLESGVPSRFSGSRSGTDFTLTISSLQPEDFATYYCQQHYTTPPTFGQGTKVEIKGSTSGSGKPGSGEGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSAAIEVMYPPPYLDNEKSNGTIIHVKGKHLCPSPLFPGPSKPFWVLVVVGGVLACYSLLVTVAFIIFWVGSGGGGSALRNVPFRSEVLGWDPDSLADYFKKLNYKDCEKAVKKYHIDGARFLNLTENDIQKFPKLRVPILSKLSQEINKNEERRSIFTRKPQVPRFPEETESHEEDNGGWSSFEEDDYESPNDDQDGEDDGDYESPNEEEEAPVEDDADYEPPPSNDEEALQNSILPAKPFPNSNSMYIDRPPSGKTPQQPPVPPQRPMAALPPPPAGRNHSPLPPPQTNHEEPSRSRNHKTAKLPAPSIDRSTKPPLDRSLAPFDREPFTLGKKPPFSDKPSIPAGRSLGEHLPKIQKPPLPPTTERHERSSPLPGKKPPVPKHGWGPDRRENDEDDVHQRPLPQPALLPMSSNTFPSRSTKPSPMNPLPSSHMPGAFSESNSSFPQSASLPPYFSQGPSNRPPIRAEGRNFPLPLPNKPRPPSPAEEENSLNEEWYVSYITRPEAEAALRKINQDGTFLVRDSSKKTTTNPYVLMVLYKDKVYNIQIRYQKESQVYLLGTGLRGKEDFLSVSDIIDYFRKMPLLLIDGKNRGSRYQCTLTHAAGYPGNS

SEQ ID NO: 20 - DNA, 1608 bp, Artificial sequence
  Synthetic polynucleotide
atggctctgagaaacgtgcccttcagatccgaggttctcggctgggaccctgatagcctggccgactacttcaagaagctgaactacaaggactgcgagaaggccgtgaagaagtaccatatcgacggcgccagattcctgaacctgaccgagaacgacatccagaagttccccaagctgagagtgcccatcctgagcaagctgagccaagagatcaacaagaacgaggaacggcggagcatcttcaccagaaagccccaggtgccaagattccccgaggaaacagagagccacgaggaagataacggcggctggtccagcttcgaagaggacgactacgagagccccaacgacgatcaggatggcgaggatgatggcgattacgagtcccctaacgaagaggaagaggcccctgttgaggacgacgccgattatgagcctcctcctagcaacgacgaagaagccctgcagaacagcatcctgcctgccaagccttttccaaacagcaacagcatgtacatcgaccggcctccaagcggcaaaacacctcagcaacctccagttcctcctcagcgacctatggctgcattgcctcctccaccagccggaagaaatcactctccactgcctccacctcagaccaatcacgaggaacccagcagaagcagaaaccacaagaccgccaagctgcccgctccatccatcgatagaagcaccaagcctccactggacagatctctggcccctttcgatagagagcccttcacactgggcaagaagcctccattcagcgacaagcctagcatccctgctggaagaagcctgggcgagcatctgcctaagatccagaaacctcctctgccacctaccaccgagagacacgagagatctagccctctgcctggcaagaaaccacctgtgcctaaacacggctggggcccagacagaagggaaaacgacgaggacgatgtgcaccagaggccattgcctcaacctgctctgctgcccatgtccagcaacacattccccagcaggtccaccaaaccttctccaatgaatcccctgcctagcagccacatgcctggcgcctttagcgagagcaatagcagctttcctcagagcgccagcctgcctccttactttagccagggacctagcaaccggccacctatcagagccgagggcagaaattttcccctgccactgcctaacaagcccagacctccatctcctgccgaggaagagaacagcctgaacgaagagtggtacgtgtcctacatcaccagacctgaagccgaggctgccctgagaaagatcaaccaggatggcacctttctcgtgcgggacagcagcaaaaagaccaccaccaatccttacgtgctgatggtgctgtacaaggacaaggtgtacaacatccagatccgctaccagaaagaaagccaggtgtacctgctcggcaccggcctgagaggcaaagaggattttctgagcgtcagcgacatcatcgactactttagaaagatgcccctgctgctgatcgacggcaagaacagaggcagcagataccagtgcacactgacacacgctgccggctatcccgggaattcc

SEQ ID NO: 21 - DNA, 786 bp, Artificial sequence
  Synthetic polynucleotide
atggaggaggccatcctggtcccctgcgtgctggggctcctgctgctgcccatcctggccatgttgatggcactgtgtgtgcactgccacagactgccaggctcctacgacagcacatcctcagatagtttgtatccaaggggcatccagttcaaacggcctcacacggttgccccctggccacctgcctacccacctgtcacctcctacccacccctgagccagccagacctgctccccatcccaagatccccgcagccccttgggggctcccaccggacgccatcttcccggcgggattctgatggtgccaacagtgtggcgagctacgagaacgagggtgcgtctggcatccgaggtgcccaggctgggtggggagtctggggtccgtcctggactaggctgacccctgtgtcgttacccccagaaccagcctgtgaggatgcggatgaggatgaggacgactatcacaacccaggctacctggtggtgcttcctgacagcaccccggccactagcactgctgccccatcagctcctgcactcagcacccctggcatccgagacagtgccttctccatggagtccattgatgattacgtgaacgttccggagagcggggagagcgcagaagcgtctctggatggcagccgggagtatgtgaatgtgtcccaggaactgcatcctggagcggctaagactgagcctgccgccctgagttcccaggaggcagaggaagtggaggaagagggggctccagattacgagaatctgcaggagctgaac

SEQ ID NO: 22 - DNA, 1527 bp, Artificial sequence
  Synthetic polynucleotide
atgggctgtggctgcagctcacacccggaagatgactggatggaaaacatcgatgtgtgtgagaactgccattatcccatagtcccactggatggcaagggcacgctgctcatccgaaatggctctgaggtgcgggacccactggttacctacgaaggctccaatccgccggcttccccactgcaagacaacctggttatcgctctgcacagctatgagccctctcacgacggagatctgggctttgagaagggggaacagctccgcatcctggagcagagcggcgagtggtggaaggcgcagtccctgaccacgggccaggaaggcttcatccccttcaattttgtggccaaagcgaacagcctggagcccgaaccctggttcttcaagaacctgagccgcaaggacgcggagcggcagctcctggcgcccgggaacactcacggctccttcctcatccgggagagcgagagcaccgcgggatcgttttcactgtcggtccgggacttcgaccagaaccagggagaggtggtgaaacattacaagatccgtaatctggacaacggtggcttctacatctcccctcgaatcacttttcccggcctgcatgaactggtccgccattacaccaatgcttcagatgggctgtgcacacggttgagccgcccctgccagacccagaagccccagaagccgtggtgggaggacgagtgggaggttcccagggagacgctgaagctggtggagcggctgggggctggacagttcggggaggtgtggatggggtactacaacgggcacacgaaggtggcggtgaagagcctgaagcagggcagcatgtccccggacgccttcctggccgaggccaacctcatgaagcagctgcaacaccagcggctggttcggctctacgctgtggtcacccaggagcccatctacatcatcactgaatacatggagaatgggagtctagtggattttctcaagaccccttcaggcatcaagttgaccatcaacaaactcctggacatggcagcccaaattgcagaaggcatggcattcattgaagagcggaattatattcatcgtgaccttcgggctgccaacattctggtgtctgacaccctgagctgcaagattgcagactttggcctagcacgcctcattgaggacaacgagtacacagccagggagggggccaagtttcccattaagtggacagcgccagaagccattaactacgggacattcaccatcaagtcagatgtgtggtcttttggcatcctgctgacggaaattgtcacccacggccgcatcccttacccagggatgaccaacccggaggtgattcagaacctggagcgaggctaccgcatggtgcgccctgacaactgtccagaggagctgtaccaactcatgaggctgtgctggaaggagcgcccagaggaccggcccacctttgactacctgcgcagtgtgctggaggacttcttcacggccacagagggccagtaccagcctcagccg

SEQ ID NO: 23 - DNA, 903 bp, Artificial sequence
  Synthetic polynucleotide
cggaagcggaggggctccggagctaccaacttcagtctgctgaaacaggccggcgacgtggaagagaaccctggccctatgggggcaggtgccaccggccgcgcaatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttcaagaggtga

SEQ ID NO: 24 - DNA, 906 bp, Artificial sequence
  Synthetic polynucleotide
agacggaagcggaggggctccggagctaccaacttcagtctgctgaaacaggccggcgacgtggaagagaaccctggccctatgggggcaggtgccaccggccgcgcaatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttcaagaggtga

SEQ ID NO: 25 - DNA, 57 bp, Artificial sequence
  Synthetic polynucleotide
tacccttatgatgtcccggattatgcctacccatacgatgttccagattacgcttaa

SEQ ID NO: 26 - DNA, 2511 bp, Artificial sequence
  Synthetic polynucleotide
atggctctgagaaacgtgcccttcagatccgaggttctcggctgggaccctgatagcctggccgactacttcaagaagctgaactacaaggactgcgagaaggccgtgaagaagtaccatatcgacggcgccagattcctgaacctgaccgagaacgacatccagaagttccccaagctgagagtgcccatcctgagcaagctgagccaagagatcaacaagaacgaggaacggcggagcatcttcaccagaaagccccaggtgccaagattccccgaggaaacagagagccacgaggaagataacggcggctggtccagcttcgaagaggacgactacgagagccccaacgacgatcaggatggcgaggatgatggcgattacgagtcccctaacgaagaggaagaggcccctgttgaggacgacgccgattatgagcctcctcctagcaacgacgaagaagccctgcagaacagcatcctgcctgccaagccttttccaaacagcaacagcatgtacatcgaccggcctccaagcggcaaaacacctcagcaacctccagttcctcctcagcgacctatggctgcattgcctcctccaccagccggaagaaatcactctccactgcctccacctcagaccaatcacgaggaacccagcagaagcagaaaccacaagaccgccaagctgcccgctccatccatcgatagaagcaccaagcctccactggacagatctctggcccctttcgatagagagcccttcacactgggcaagaagcctccattcagcgacaagcctagcatccctgctggaagaagcctgggcgagcatctgcctaagatccagaaacctcctctgccacctaccaccgagagacacgagagatctagccctctgcctggcaagaaaccacctgtgcctaaacacggctggggcccagacagaagggaaaacgacgaggacgatgtgcaccagaggccattgcctcaacctgctctgctgcccatgtccagcaacacattccccagcaggtccaccaaaccttctccaatgaatcccctgcctagcagccacatgcctggcgcctttagcgagagcaatagcagctttcctcagagcgccagcctgcctccttactttagccagggacctagcaaccggccacctatcagagccgagggcagaaattttcccctgccactgcctaacaagcccagacctccatctcctgccgaggaagagaacagcctgaacgaagagtggtacgtgtcctacatcaccagacctgaagccgaggctgccctgagaaagatcaaccaggatggcacctttctcgtgcgggacagcagcaaaaagaccaccaccaatccttacgtgctgatggtgctgtacaaggacaaggtgtacaacatccagatccgctaccagaaagaaagccaggtgtacctgctcggcaccggcctgagaggcaaagaggattttctgagcgtcagcgacatcatcgactactttagaaagatgcccctgctgctgatcgacggcaagaacagaggcagcagataccagtgcacactgacacacgctgccggctatcccgggaattcccggaagcggaggggctccggagctaccaacttcagtctgctgaaacaggccggcgacgtggaagagaaccctggccctatgggggcaggtgccaccggccgcgcaatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttcaagaggtga

SEQ ID NO: 27 - DNA, 1692 bp, Artificial sequence
  Synthetic polynucleotide
atggaggaggccatcctggtcccctgcgtgctggggctcctgctgctgcccatcctggccatgttgatggcactgtgtgtgcactgccacagactgccaggctcctacgacagcacatcctcagatagtttgtatccaaggggcatccagttcaaacggcctcacacggttgccccctggccacctgcctacccacctgtcacctcctacccacccctgagccagccagacctgctccccatcccaagatccccgcagccccttgggggctcccaccggacgccatcttcccggcgggattctgatggtgccaacagtgtggcgagctacgagaacgagggtgcgtctggcatccgaggtgcccaggctgggtggggagtctggggtccgtcctggactaggctgacccctgtgtcgttacccccagaaccagcctgtgaggatgcggatgaggatgaggacgactatcacaacccaggctacctggtggtgcttcctgacagcaccccggccactagcactgctgccccatcagctcctgcactcagcacccctggcatccgagacagtgccttctccatggagtccattgatgattacgtgaacgttccggagagcggggagagcgcagaagcgtctctggatggcagccgggagtatgtgaatgtgtcccaggaactgcatcctggagcggctaagactgagcctgccgccctgagttcccaggaggcagaggaagtggaggaagagggggctccagattacgagaatctgcaggagctgaacagacggaagcggaggggctccggagctaccaacttcagtctgctgaaacaggccggcgacgtggaagagaaccctggccctatgggggcaggtgccaccggccgcgcaatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttcaagaggtga

SEQ ID NO: 28 - DNA, 1584 bp, Artificial sequence
  Synthetic polynucleotide
atgggctgtggctgcagctcacacccggaagatgactggatggaaaacatcgatgtgtgtgagaactgccattatcccatagtcccactggatggcaagggcacgctgctcatccgaaatggctctgaggtgcgggacccactggttacctacgaaggctccaatccgccggcttccccactgcaagacaacctggttatcgctctgcacagctatgagccctctcacgacggagatctgggctttgagaagggggaacagctccgcatcctggagcagagcggcgagtggtggaaggcgcagtccctgaccacgggccaggaaggcttcatccccttcaattttgtggccaaagcgaacagcctggagcccgaaccctggttcttcaagaacctgagccgcaaggacgcggagcggcagctcctggcgcccgggaacactcacggctccttcctcatccgggagagcgagagcaccgcgggatcgttttcactgtcggtccgggacttcgaccagaaccagggagaggtggtgaaacattacaagatccgtaatctggacaacggtggcttctacatctcccctcgaatcacttttcccggcctgcatgaactggtccgccattacaccaatgcttcagatgggctgtgcacacggttgagccgcccctgccagacccagaagccccagaagccgtggtgggaggacgagtgggaggttcccagggagacgctgaagctggtggagcggctgggggctggacagttcggggaggtgtggatggggtactacaacgggcacacgaaggtggcggtgaagagcctgaagcagggcagcatgtccccggacgccttcctggccgaggccaacctcatgaagcagctgcaacaccagcggctggttcggctctacgctgtggtcacccaggagcccatctacatcatcactgaatacatggagaatgggagtctagtggattttctcaagaccccttcaggcatcaagttgaccatcaacaaactcctggacatggcagcccaaattgcagaaggcatggcattcattgaagagcggaattatattcatcgtgaccttcgggctgccaacattctggtgtctgacaccctgagctgcaagattgcagactttggcctagcacgcctcattgaggacaacgagtacacagccagggagggggccaagtttcccattaagtggacagcgccagaagccattaactacgggacattcaccatcaagtcagatgtgtggtcttttggcatcctgctgacggaaattgtcacccacggccgcatcccttacccagggatgaccaacccggaggtgattcagaacctggagcgaggctaccgcatggtgcgccctgacaactgtccagaggagctgtaccaactcatgaggctgtgctggaaggagcgcccagaggaccggcccacctttgactacctgcgcagtgtgctggaggacttcttcacggccacagagggccagtaccagcctcagccgtacccttatgatgtcccggattatgcctacccatacgatgttccagattacgcttaa

SEQ ID NO: 29 - DNA, 96 bp, Artificial sequence
  Synthetic polynucleotide
atggctcgctcggtgaccctggtctttctggtgcttgtctcactgaccggtttgtatgctgcttacactgatatcgaaatgaaccgcctgggtaag

SEQ ID NO: 30 - DNA, 792 bp, Artificial sequence
  Synthetic polynucleotide
atggctcgctcggtgaccctggtctttctggtgcttgtctcactgaccggtttgtatgctgctgatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttccttgagtctggagtcccttctcgcttctctggatctagatctgggacggatttcactctgaccatcagcagtctgcagccggaagacttcgcaacttattactgtcagcaacattatactactcctcccacgttcggacagggtaccaaggtggagatcaaagggtctacatctggatctgggaagccgggttctggtgagggttctggtgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggacgtgtggggtcaaggaaccctggtcaccgtctcctcg

SEQ ID NO: 31 - DNA, 216 bp, Artificial sequence
  Synthetic polynucleotide
gccgcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaa

SEQ ID NO: 32 - DNA, 204 bp, Artificial sequence
  Synthetic polynucleotide
gccgcaatcgaagtgatgtaccctccaccttacctggacaacgagaagtccaacggcaccatcatccacgtgaagggcaagcacctgtgtccttctccactgttccctggacctagcaagcctttctgggtgctcgtggttgttggcggcgtgctggcctgttattctctgctggtcaccgtggccttcatcatcttttgggtc

SEQ ID NO: 33 - DNA, 1629 bp, Artificial sequence
  Synthetic polynucleotide
ggatccggcggtggtggatctgctctgagaaacgtgcccttcagatccgaggttctcggctgggaccctgatagcctggccgactacttcaagaagctgaactacaaggactgcgagaaggccgtgaagaagtaccatatcgacggcgccagattcctgaacctgaccgagaacgacatccagaagttccccaagctgagagtgcccatcctgagcaagctgagccaagagatcaacaagaacgaggaacggcggagcatcttcaccagaaagccccaggtgccaagattccccgaggaaacagagagccacgaggaagataacggcggctggtccagcttcgaagaggacgactacgagagccccaacgacgatcaggatggcgaggatgatggcgattacgagtcccctaacgaagaggaagaggcccctgttgaggacgacgccgattatgagcctcctcctagcaacgacgaagaagccctgcagaacagcatcctgcctgccaagccttttccaaacagcaacagcatgtacatcgaccggcctccaagcggcaaaacacctcagcaacctccagttcctcctcagcgacctatggctgcattgcctcctccaccagccggaagaaatcactctccactgcctccacctcagaccaatcacgaggaacccagcagaagcagaaaccacaagaccgccaagctgcccgctccatccatcgatagaagcaccaagcctccactggacagatctctggcccctttcgatagagagcccttcacactgggcaagaagcctccattcagcgacaagcctagcatccctgctggaagaagcctgggcgagcatctgcctaagatccagaaacctcctctgccacctaccaccgagagacacgagagatctagccctctgcctggcaagaaaccacctgtgcctaaacacggctggggcccagacagaagggaaaacgacgaggacgatgtgcaccagaggccattgcctcaacctgctctgctgcccatgtccagcaacacattccccagcaggtccaccaaaccttctccaatgaatcccctgcctagcagccacatgcctggcgcctttagcgagagcaatagcagctttcctcagagcgccagcctgcctccttactttagccagggacctagcaaccggccacctatcagagccgagggcagaaattttcccctgccactgcctaacaagcccagacctccatctcctgccgaggaagagaacagcctgaacgaagagtggtacgtgtcctacatcaccagacctgaagccgaggctgccctgagaaagatcaaccaggatggcacctttctcgtgcgggacagcagcaaaaagaccaccaccaatccttacgtgctgatggtgctgtacaaggacaaggtgtacaacatccagatccgctaccagaaagaaagccaggtgtacctgctcggcaccggcctgagaggcaaagaggattttctgagcgtcagcgacatcatcgactactttagaaagatgcccctgctgctgatcgacggcaagaacagaggcagcagataccagtgcacactgacacacgctgccggctatcccgggaattcctga

SEQ ID NO: 34 - DNA, 1434 bp, Artificial sequence
  Synthetic polynucleotide
ggatccatccggaacggctctgaagttcgcgatcctctggtcacctacgagggcagcaatcctcctgccagtcctctgcaggataacctcgtgatcgccctgcacagctacgagccttctcatgatggcgacctgggctttgagaagggcgagcagctgagaatcctggaacagtctggcgagtggtggaaggcccagtctctgacaacaggccaagagggcttcatccccttcaacttcgtggccaaggccaacagcctggaacctgagccttggtttttcaagaacctgtccagaaaggacgccgagagacagctgctggcccctggaaatacccacggcagctttctgattagagagagcgagagcacagccggcagcttcagcctgagtgtgcgggactttgaccagaatcaaggcgaggtcgtgaagcactacaagatccggaacctggataacggcggcttctacatcagccccagaatcacctttcctggcctgcacgaactcgtgcggcactacacaaatgcctccgatggcctgtgcaccagactgagcagaccctgtcagacccagaagcctcagaaaccttggtgggaagatgagtgggaagtgcccagagaaaccctgaagctggtggaaagactcggagccggccagtttggagaagtgtggatgggctactacaatggccacaccaaggtggccgtgaagtctctgaagcagggctctatgagcccagatgcttttctggccgaggccaatctgatgaagcagctgcagcaccagagacttgtgcggctgtatgccgtggtcacccaagagcctatctacatcatcaccgagtacatggaaaacggcagcctggtggacttcctgaaaacccctagcggcatcaagctgaccattaacaagctgctggacatggccgctcagatcgccgaaggcatggcctttatcgaggaacggaactacatccacagggacctgagagccgccaatatcctggtgtctgacaccctgagctgcaagattgccgatttcggcctggccagactgatcgaggacaatgagtataccgccagagagggcgccaagtttcccatcaaatggacagcccctgaggccatcaactacggcaccttcaccatcaagagcgacgtgtggtccttcggcatcctgctgacagagatcgtgacacacggcagaatcccatatcctggcatgacaaaccccgaagtgatccagaatctggaacggggctacagaatggtccgacctgacaactgccccgaggaactgtaccagctgatgagactgtgctggaaagagaggcccgaggacagacccaccttcgactacctgagaagcgtgctggaagatttcttcaccgccaccgagggccagtaccagcctcagccagggaattcctga

SEQ ID NO: 35 - DNA, 1941 bp, Artificial sequence
  Synthetic polynucleotide
atggctcgctcggtgaccctggtctttctggtgcttgtctcactgaccggtttgtatgctgcttacactgatatcgaaatgaaccgcctgggtaaggccgcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaaggatccggcggtggtggatctgctctgagaaacgtgcccttcagatccgaggttctcggctgggaccctgatagcctggccgactacttcaagaagctgaactacaaggactgcgagaaggccgtgaagaagtaccatatcgacggcgccagattcctgaacctgaccgagaacgacatccagaagttccccaagctgagagtgcccatcctgagcaagctgagccaagagatcaacaagaacgaggaacggcggagcatcttcaccagaaagccccaggtgccaagattccccgaggaaacagagagccacgaggaagataacggcggctggtccagcttcgaagaggacgactacgagagccccaacgacgatcaggatggcgaggatgatggcgattacgagtcccctaacgaagaggaagaggcccctgttgaggacgacgccgattatgagcctcctcctagcaacgacgaagaagccctgcagaacagcatcctgcctgccaagccttttccaaacagcaacagcatgtacatcgaccggcctccaagcggcaaaacacctcagcaacctccagttcctcctcagcgacctatggctgcattgcctcctccaccagccggaagaaatcactctccactgcctccacctcagaccaatcacgaggaacccagcagaagcagaaaccacaagaccgccaagctgcccgctccatccatcgatagaagcaccaagcctccactggacagatctctggcccctttcgatagagagcccttcacactgggcaagaagcctccattcagcgacaagcctagcatccctgctggaagaagcctgggcgagcatctgcctaagatccagaaacctcctctgccacctaccaccgagagacacgagagatctagccctctgcctggcaagaaaccacctgtgcctaaacacggctggggcccagacagaagggaaaacgacgaggacgatgtgcaccagaggccattgcctcaacctgctctgctgcccatgtccagcaacacattccccagcaggtccaccaaaccttctccaatgaatcccctgcctagcagccacatgcctggcgcctttagcgagagcaatagcagctttcctcagagcgccagcctgcctccttactttagccagggacctagcaaccggccacctatcagagccgagggcagaaattttcccctgccactgcctaacaagcccagacctccatctcctgccgaggaagagaacagcctgaacgaagagtggtacgtgtcctacatcaccagacctgaagccgaggctgccctgagaaagatcaaccaggatggcacctttctcgtgcgggacagcagcaaaaagaccaccaccaatccttacgtgctgatggtgctgtacaaggacaaggtgtacaacatccagatccgctaccagaaagaaagccaggtgtacctgctcggcaccggcctgagaggcaaagaggattttctgagcgtcagcgacatcatcgactactttagaaagatgcccctgctgctgatcgacggcaagaacagaggcagcagataccagtgcacactgacacacgctgccggctatcccgggaattcctga

SEQ ID NO: 36 - DNA, 1746 bp, Artificial sequence
  Synthetic polynucleotide
atggctcgctcggtgaccctggtctttctggtgcttgtctcactgaccggtttgtatgctgcttacactgatatcgaaatgaaccgcctgggtaaggccgcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaaggatccatccggaacggctctgaagttcgcgatcctctggtcacctacgagggcagcaatcctcctgccagtcctctgcaggataacctcgtgatcgccctgcacagctacgagccttctcatgatggcgacctgggctttgagaagggcgagcagctgagaatcctggaacagtctggcgagtggtggaaggcccagtctctgacaacaggccaagagggcttcatccccttcaacttcgtggccaaggccaacagcctggaacctgagccttggtttttcaagaacctgtccagaaaggacgccgagagacagctgctggcccctggaaatacccacggcagctttctgattagagagagcgagagcacagccggcagcttcagcctgagtgtgcgggactttgaccagaatcaaggcgaggtcgtgaagcactacaagatccggaacctggataacggcggcttctacatcagccccagaatcacctttcctggcctgcacgaactcgtgcggcactacacaaatgcctccgatggcctgtgcaccagactgagcagaccctgtcagacccagaagcctcagaaaccttggtgggaagatgagtgggaagtgcccagagaaaccctgaagctggtggaaagactcggagccggccagtttggagaagtgtggatgggctactacaatggccacaccaaggtggccgtgaagtctctgaagcagggctctatgagcccagatgcttttctggccgaggccaatctgatgaagcagctgcagcaccagagacttgtgcggctgtatgccgtggtcacccaagagcctatctacatcatcaccgagtacatggaaaacggcagcctggtggacttcctgaaaacccctagcggcatcaagctgaccattaacaagctgctggacatggccgctcagatcgccgaaggcatggcctttatcgaggaacggaactacatccacagggacctgagagccgccaatatcctggtgtctgacaccctgagctgcaagattgccgatttcggcctggccagactgatcgaggacaatgagtataccgccagagagggcgccaagtttcccatcaaatggacagcccctgaggccatcaactacggcaccttcaccatcaagagcgacgtgtggtccttcggcatcctgctgacagagatcgtgacacacggcagaatcccatatcctggcatgacaaaccccgaagtgatccagaatctggaacggggctacagaatggtccgacctgacaactgccccgaggaactgtaccagctgatgagactgtgctggaaagagaggcccgaggacagacccaccttcgactacctgagaagcgtgctggaagatttcttcaccgccaccgagggccagtaccagcctcagccagggaattcctga

SEQ ID NO: 37 - DNA, 2637 bp, Artificial sequence
  Synthetic polynucleotide
atggctcgctcggtgaccctggtctttctggtgcttgtctcactgaccggtttgtatgctgctgatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttccttgagtctggagtcccttctcgcttctctggatctagatctgggacggatttcactctgaccatcagcagtctgcagccggaagacttcgcaacttattactgtcagcaacattatactactcctcccacgttcggacagggtaccaaggtggagatcaaagggtctacatctggatctgggaagccgggttctggtgagggttctggtgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggacgtgtggggtcaaggaaccctggtcaccgtctcctcggccgcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaaggatccggcggtggtggatctgctctgagaaacgtgcccttcagatccgaggttctcggctgggaccctgatagcctggccgactacttcaagaagctgaactacaaggactgcgagaaggccgtgaagaagtaccatatcgacggcgccagattcctgaacctgaccgagaacgacatccagaagttccccaagctgagagtgcccatcctgagcaagctgagccaagagatcaacaagaacgaggaacggcggagcatcttcaccagaaagccccaggtgccaagattccccgaggaaacagagagccacgaggaagataacggcggctggtccagcttcgaagaggacgactacgagagccccaacgacgatcaggatggcgaggatgatggcgattacgagtcccctaacgaagaggaagaggcccctgttgaggacgacgccgattatgagcctcctcctagcaacgacgaagaagccctgcagaacagcatcctgcctgccaagccttttccaaacagcaacagcatgtacatcgaccggcctccaagcggcaaaacacctcagcaacctccagttcctcctcagcgacctatggctgcattgcctcctccaccagccggaagaaatcactctccactgcctccacctcagaccaatcacgaggaacccagcagaagcagaaaccacaagaccgccaagctgcccgctccatccatcgatagaagcaccaagcctccactggacagatctctggcccctttcgatagagagcccttcacactgggcaagaagcctccattcagcgacaagcctagcatccctgctggaagaagcctgggcgagcatctgcctaagatccagaaacctcctctgccacctaccaccgagagacacgagagatctagccctctgcctggcaagaaaccacctgtgcctaaacacggctggggcccagacagaagggaaaacgacgaggacgatgtgcaccagaggccattgcctcaacctgctctgctgcccatgtccagcaacacattccccagcaggtccaccaaaccttctccaatgaatcccctgcctagcagccacatgcctggcgcctttagcgagagcaatagcagctttcctcagagcgccagcctgcctccttactttagccagggacctagcaaccggccacctatcagagccgagggcagaaattttcccctgccactgcctaacaagcccagacctccatctcctgccgaggaagagaacagcctgaacgaagagtggtacgtgtcctacatcaccagacctgaagccgaggctgccctgagaaagatcaaccaggatggcacctttctcgtgcgggacagcagcaaaaagaccaccaccaatccttacgtgctgatggtgctgtacaaggacaaggtgtacaacatccagatccgctaccagaaagaaagccaggtgtacctgctcggcaccggcctgagaggcaaagaggattttctgagcgtcagcgacatcatcgactactttagaaagatgcccctgctgctgatcgacggcaagaacagaggcagcagataccagtgcacactgacacacgctgccggctatcccgggaattcctga

SEQ ID NO: 38 - DNA, 2625 bp, Artificial sequence
  Synthetic polynucleotide
atggctcgctcggtgaccctggtctttctggtgcttgtctcactgaccggtttgtatgctgctgatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttccttgagtctggagtcccttctcgcttctctggatctagatctgggacggatttcactctgaccatcagcagtctgcagccggaagacttcgcaacttattactgtcagcaacattatactactcctcccacgttcggacagggtaccaaggtggagatcaaagggtctacatctggatctgggaagccgggttctggtgagggttctggtgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggacgtgtggggtcaaggaaccctggtcaccgtctcctcggccgcaatcgaagtgatgtaccctccaccttacctggacaacgagaagtccaacggcaccatcatccacgtgaagggcaagcacctgtgtccttctccactgttccctggacctagcaagcctttctgggtgctcgtggttgttggcggcgtgctggcctgttattctctgctggtcaccgtggccttcatcatcttttgggtcggatccggcggtggtggatctgctctgagaaacgtgcccttcagatccgaggttctcggctgggaccctgatagcctggccgactacttcaagaagctgaactacaaggactgcgagaaggccgtgaagaagtaccatatcgacggcgccagattcctgaacctgaccgagaacgacatccagaagttccccaagctgagagtgcccatcctgagcaagctgagccaagagatcaacaagaacgaggaacggcggagcatcttcaccagaaagccccaggtgccaagattccccgaggaaacagagagccacgaggaagataacggcggctggtccagcttcgaagaggacgactacgagagccccaacgacgatcaggatggcgaggatgatggcgattacgagtcccctaacgaagaggaagaggcccctgttgaggacgacgccgattatgagcctcctcctagcaacgacgaagaagccctgcagaacagcatcctgcctgccaagccttttccaaacagcaacagcatgtacatcgaccggcctccaagcggcaaaacacctcagcaacctccagttcctcctcagcgacctatggctgcattgcctcctccaccagccggaagaaatcactctccactgcctccacctcagaccaatcacgaggaacccagcagaagcagaaaccacaagaccgccaagctgcccgctccatccatcgatagaagcaccaagcctccactggacagatctctggcccctttcgatagagagcccttcacactgggcaagaagcctccattcagcgacaagcctagcatccctgctggaagaagcctgggcgagcatctgcctaagatccagaaacctcctctgccacctaccaccgagagacacgagagatctagccctctgcctggcaagaaaccacctgtgcctaaacacggctggggcccagacagaagggaaaacgacgaggacgatgtgcaccagaggccattgcctcaacctgctctgctgcccatgtccagcaacacattccccagcaggtccaccaaaccttctccaatgaatcccctgcctagcagccacatgcctggcgcctttagcgagagcaatagcagctttcctcagagcgccagcctgcctccttactttagccagggacctagcaaccggccacctatcagagccgagggcagaaattttcccctgccactgcctaacaagcccagacctccatctcctgccgaggaagagaacagcctgaacgaagagtggtacgtgtcctacatcaccagacctgaagccgaggctgccctgagaaagatcaaccaggatggcacctttctcgtgcgggacagcagcaaaaagaccaccaccaatccttacgtgctgatggtgctgtacaaggacaaggtgtacaacatccagatccgctaccagaaagaaagccaggtgtacctgctcggcaccggcctgagaggcaaagaggattttctgagcgtcagcgacatcatcgactactttagaaagatgcccctgctgctgatcgacggcaagaacagaggcagcagataccagtgcacactgacacacgctgccggctatcccgggaattcctga

SEQ ID NO: 39 - protein, 267 aa, Artificial sequence
  Synthetic polypeptide
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 40 - protein, 881 aa, Artificial sequence
  Synthetic polypeptide
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAATTTPAPRPPTPAPTIASQPLSLRPEACRPAAGGAVHTRGLDFACDIYIWAPLAGTCGVLLLSLVITLYCKGSGGGGSALRNVPFRSEVLGWDPDSLADYFKKLNYKDCEKAVKKYHIDGARFLNLTENDIQKFPKLRVPILSKLSQEINKNEERRSIFTRKPQVPRFPEETESHEEDNGGWSSFEEDDYESPNDDQDGEDDGDYESPNEEEEAPVEDDADYEPPPSNDEEALQNSILPAKPFPNSNSMYIDRPPSGKTPQQPPVPPQRPMAALPPPPAGRNHSPLPPPQTNHEEPSRSRNHKTAKLPAPSIDRSTKPPLDRSLAPFDREPFTLGKKPPFSDKPSIPAGRSLGEHLPKIQKPPLPPTTERHERSSPLPGKKPPVPKHGWGPDRRENDEDDVHQRPLPQPALLPMSSNTFPSRSTKPSPMNPLPSSHMPGAFSESNSSFPQSASLPPYFSQGPSNRPPIRAEGRNFPLPLPNKPRPPSPAEEENSLNEEWYVSYITRPEAEAALRKINQDGTFLVRDSSKKTTTNPYVLMVLYKDKVYNIQIRYQKESQVYLLGTGLRGKEDFLSVSDIIDYFRKMPLLLIDGKNRGSRYQCTLTHAAGYPGNS

SEQ ID NO: 41 - DNA, 801 bp, Artificial sequence
  Synthetic polynucleotide
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctca

SEQ ID NO: 42 - DNA, 2646 bp, Artificial sequence
  Synthetic polynucleotide
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagccgcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaaggatccggcggtggtggatctgctctgagaaacgtgcccttcagatccgaggttctcggctgggaccctgatagcctggccgactacttcaagaagctgaactacaaggactgcgagaaggccgtgaagaagtaccatatcgacggcgccagattcctgaacctgaccgagaacgacatccagaagttccccaagctgagagtgcccatcctgagcaagctgagccaagagatcaacaagaacgaggaacggcggagcatcttcaccagaaagccccaggtgccaagattccccgaggaaacagagagccacgaggaagataacggcggctggtccagcttcgaagaggacgactacgagagccccaacgacgatcaggatggcgaggatgatggcgattacgagtcccctaacgaagaggaagaggcccctgttgaggacgacgccgattatgagcctcctcctagcaacgacgaagaagccctgcagaacagcatcctgcctgccaagccttttccaaacagcaacagcatgtacatcgaccggcctccaagcggcaaaacacctcagcaacctccagttcctcctcagcgacctatggctgcattgcctcctccaccagccggaagaaatcactctccactgcctccacctcagaccaatcacgaggaacccagcagaagcagaaaccacaagaccgccaagctgcccgctccatccatcgatagaagcaccaagcctccactggacagatctctggcccctttcgatagagagcccttcacactgggcaagaagcctccattcagcgacaagcctagcatccctgctggaagaagcctgggcgagcatctgcctaagatccagaaacctcctctgccacctaccaccgagagacacgagagatctagccctctgcctggcaagaaaccacctgtgcctaaacacggctggggcccagacagaagggaaaacgacgaggacgatgtgcaccagaggccattgcctcaacctgctctgctgcccatgtccagcaacacattccccagcaggtccaccaaaccttctccaatgaatcccctgcctagcagccacatgcctggcgcctttagcgagagcaatagcagctttcctcagagcgccagcctgcctccttactttagccagggacctagcaaccggccacctatcagagccgagggcagaaattttcccctgccactgcctaacaagcccagacctccatctcctgccgaggaagagaacagcctgaacgaagagtggtacgtgtcctacatcaccagacctgaagccgaggctgccctgagaaagatcaaccaggatggcacctttctcgtgcgggacagcagcaaaaagaccaccaccaatccttacgtgctgatggtgctgtacaaggacaaggtgtacaacatccagatccgctaccagaaagaaagccaggtgtacctgctcggcaccggcctgagaggcaaagaggattttctgagcgtcagcgacatcatcgactactttagaaagatgcccctgctgctgatcgacggcaagaacagaggcagcagataccagtgcacactgacacacgctgccggctatcccgggaattcctga

SEQ ID NO: 43 - protein, 619 aa, Artificial sequence
  Synthetic polypeptide
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 44 - protein, 919 aa, Artificial sequence
  Synthetic polypeptide
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACARKRRGSGATNFSLLKQAGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 45 - protein, 351 aa, Artificial sequence
  Synthetic polypeptide
GSPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLGNS

SEQ ID NO: 46 - protein, 455 aa, Artificial sequence
  Synthetic polypeptide
MARSVTLVFLVLVSLTGLYAAYTDIEMNRLGKAATTTPAPRPPTPAPTIASQPLSLRPEACRPAAGGAVHTRGLDFACDIYIWAPLAGTCGVLLLSLVITLYCKGSPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLGNS

SEQ ID NO: 47 - protein, 1290 aa, Artificial sequence
  Synthetic polypeptide
MAGAASPCANGCGPGAPSDAEVLHLCRSLEVGTVMTLFYSKKSQRPERKTFQVKLETRQITWSRGADKIEGAIDIREIKEIRPGKTSRDFDRYQEDPAFRPDQSHCFVILYGMEFRLKTLSLQATSEDEVNMWIKGLTWLMEDTLQAPTPLQIERWLRKQFYSVDRNREDRISAKDLKNMLSQVNYRVPNMRFLRERLTDLEQRSGDITYGQFAQLYRSLMYSAQKTMDLPFLEASTLRAGERPELCRVSLPEFQQFLLDYQGELWAVDRLQVQEFMLSFLRDPLREIEEPYFFLDEFVTFLFSKENSVWNSQLDAVCPDTMNNPLSHYWISSSHNTYLTGDQFSSESSLEAYARCLRMGCRCIELDCWDGPDGMPVIYHGHTLTTKIKFSDVLHTIKEHAFVASEYPVILSIEDHCSIAQQRNMAQYFKKVLGDTLLTKPVEISADGLPSPNQLKRKILIKHKKLAEGSAYEEVPTSMMYSENDISNSIKNGILYLEDPVNHEWYPHYFVLTSSKIYYSEETSSDQGNEDEEEPKEVSSSTELHSNEKWFHGKLGAGRDGRHIAERLLTEYCIETGAPDGSFLVRESETFVGDYTLSFWRNGKVQHCRIHSRQDAGTPKFFLTDNLVFDSLYDLITHYQQVPLRCNEFEMRLSEPVPQTNAHESKEWYHASLTRAQAEHMLMRVPRDGAFLVRKRNEPNSYAISFRAEGKIKHCRVQQEGQTVMLGNSEFDSLVDLISYYEKHPLYRKMKLRYPINEEALEKIGTAEPDYGALYEGRNPGFYVEANPMPTFKCAVKALFDYKAQREDELTFIKSAIIQNVEKQEGGWWRGDYGGKKQLWFPSNYVEEMVNPVALEPEREHLDENSPLGDLLRGVLDVPACQIAIRPEGKNNRLFVFSISMASVAHWSLDVAADSQEELQDWVKKIREVAQTADARLTEGKIMERRKKIALELSELVVYCRPVPFDEEKIGTERACYRDMSSFPETKAEKYVNKAKGKKFLQYNRLQLSRIYPKGQRLDSSNYDPLPMWICGSQLVALNFQTPDKPMQMNQALFMTGRHCGYVLQPSTMRDEAFDPFDKSSLRGLEPCAISIEVLGARHLPKNGRGIVCPFVEIEVAGAEYDSTKQKTEFVVDNGLNPVWPAKPFHFQISNPEFAFLRFVVYEEDMFSDQNFLAQATFPVKGLKTGYRAVPLKNNYSEDLELASLLIKIDIFPAKENGDLSPFSGTSLRERGSDASGQLFHGRAREGSFESRYQQPFEDFRISQEHLADHFDSRERRAPRRTRVNGDNRL

SEQ ID NO: 48 - protein, 1590 aa, Artificial sequence
  Synthetic polypeptide
MAGAASPCANGCGPGAPSDAEVLHLCRSLEVGTVMTLFYSKKSQRPERKTFQVKLETRQITWSRGADKIEGAIDIREIKEIRPGKTSRDFDRYQEDPAFRPDQSHCFVILYGMEFRLKTLSLQATSEDEVNMWIKGLTWLMEDTLQAPTPLQIERWLRKQFYSVDRNREDRISAKDLKNMLSQVNYRVPNMRFLRERLTDLEQRSGDITYGQFAQLYRSLMYSAQKTMDLPFLEASTLRAGERPELCRVSLPEFQQFLLDYQGELWAVDRLQVQEFMLSFLRDPLREIEEPYFFLDEFVTFLFSKENSVWNSQLDAVCPDTMNNPLSHYWISSSHNTYLTGDQFSSESSLEAYARCLRMGCRCIELDCWDGPDGMPVIYHGHTLTTKIKFSDVLHTIKEHAFVASEYPVILSIEDHCSIAQQRNMAQYFKKVLGDTLLTKPVEISADGLPSPNQLKRKILIKHKKLAEGSAYEEVPTSMMYSENDISNSIKNGILYLEDPVNHEWYPHYFVLTSSKIYYSEETSSDQGNEDEEEPKEVSSSTELHSNEKWFHGKLGAGRDGRHIAERLLTEYCIETGAPDGSFLVRESETFVGDYTLSFWRNGKVQHCRIHSRQDAGTPKFFLTDNLVFDSLYDLITHYQQVPLRCNEFEMRLSEPVPQTNAHESKEWYHASLTRAQAEHMLMRVPRDGAFLVRKRNEPNSYAISFRAEGKIKHCRVQQEGQTVMLGNSEFDSLVDLISYYEKHPLYRKMKLRYPINEEALEKIGTAEPDYGALYEGRNPGFYVEANPMPTFKCAVKALFDYKAQREDELTFIKSAIIQNVEKQEGGWWRGDYGGKKQLWFPSNYVEEMVNPVALEPEREHLDENSPLGDLLRGVLDVPACQIAIRPEGKNNRLFVFSISMASVAHWSLDVAADSQEELQDWVKKIREVAQTADARLTEGKIMERRKKIALELSELVVYCRPVPFDEEKIGTERACYRDMSSFPETKAEKYVNKAKGKKFLQYNRLQLSRIYPKGQRLDSSNYDPLPMWICGSQLVALNFQTPDKPMQMNQALFMTGRHCGYVLQPSTMRDEAFDPFDKSSLRGLEPCAISIEVLGARHLPKNGRGIVCPFVEIEVAGAEYDSTKQKTEFVVDNGLNPVWPAKPFHFQISNPEFAFLRFVVYEEDMFSDQNFLAQATFPVKGLKTGYRAVPLKNNYSEDLELASLLIKIDIFPAKENGDLSPFSGTSLRERGSDASGQLFHGRAREGSFESRYQQPFEDFRISQEHLADHFDSRERRAPRRTRVNGDNRLRKRRGSGATNFSLLKQAGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 49 - protein, 1296 aa, Artificial sequence
  Synthetic polypeptide
GSGGGGSAGAASPCANGCGPGAPSDAEVLHLCRSLEVGTVMTLFYSKKSQRPERKTFQVKLETRQITWSRGADKIEGAIDIREIKEIRPGKTSRDFDRYQEDPAFRPDQSHCFVILYGMEFRLKTLSLQATSEDEVNMWIKGLTWLMEDTLQAPTPLQIERWLRKQFYSVDRNREDRISAKDLKNMLSQVNYRVPNMRFLRERLTDLEQRSGDITYGQFAQLYRSLMYSAQKTMDLPFLEASTLRAGERPELCRVSLPEFQQFLLDYQGELWAVDRLQVQEFMLSFLRDPLREIEEPYFFLDEFVTFLFSKENSVWNSQLDAVCPDTMNNPLSHYWISSSHNTYLTGDQFSSESSLEAYARCLRMGCRCIELDCWDGPDGMPVIYHGHTLTTKIKFSDVLHTIKEHAFVASEYPVILSIEDHCSIAQQRNMAQYFKKVLGDTLLTKPVEISADGLPSPNQLKRKILIKHKKLAEGSAYEEVPTSMMYSENDISNSIKNGILYLEDPVNHEWYPHYFVLTSSKIYYSEETSSDQGNEDEEEPKEVSSSTELHSNEKWFHGKLGAGRDGRHIAERLLTEYCIETGAPDGSFLVRESETFVGDYTLSFWRNGKVQHCRIHSRQDAGTPKFFLTDNLVFDSLYDLITHYQQVPLRCNEFEMRLSEPVPQTNAHESKEWYHASLTRAQAEHMLMRVPRDGAFLVRKRNEPNSYAISFRAEGKIKHCRVQQEGQTVMLGNSEFDSLVDLISYYEKHPLYRKMKLRYPINEEALEKIGTAEPDYGALYEGRNPGFYVEANPMPTFKCAVKALFDYKAQREDELTFIKSAIIQNVEKQEGGWWRGDYGGKKQLWFPSNYVEEMVNPVALEPEREHLDENSPLGDLLRGVLDVPACQIAIRPEGKNNRLFVFSISMASVAHWSLDVAADSQEELQDWVKKIREVAQTADARLTEGKIMERRKKIALELSELVVYCRPVPFDEEKIGTERACYRDMSSFPETKAEKYVNKAKGKKFLQYNRLQLSRIYPKGQRLDSSNYDPLPMWICGSQLVALNFQTPDKPMQMNQALFMTGRHCGYVLQPSTMRDEAFDPFDKSSLRGLEPCAISIEVLGARHLPKNGRGIVCPFVEIEVAGAEYDSTKQKTEFVVDNGLNPVWPAKPFHFQISNPEFAFLRFVVYEEDMFSDQNFLAQATFPVKGLKTGYRAVPLKNNYSEDLELASLLIKIDIFPAKENGDLSPFSGTSLRERGSDASGQLFHGRAREGSFESRYQQPFEDFRISQEHLADHFDSRERRAPRRTRVNGDNRL

SEQ ID NO: 50 - protein, 1400 aa, Artificial sequence
  Synthetic polypeptide
MARSVTLVFLVLVSLTGLYAAYTDIEMNRLGKAATTTPAPRPPTPAPTIASQPLSLRPEACRPAAGGAVHTRGLDFACDIYIWAPLAGTCGVLLLSLVITLYCKGSGGGGSAGAASPCANGCGPGAPSDAEVLHLCRSLEVGTVMTLFYSKKSQRPERKTFQVKLETRQITWSRGADKIEGAIDIREIKEIRPGKTSRDFDRYQEDPAFRPDQSHCFVILYGMEFRLKTLSLQATSEDEVNMWIKGLTWLMEDTLQAPTPLQIERWLRKQFYSVDRNREDRISAKDLKNMLSQVNYRVPNMRFLRERLTDLEQRSGDITYGQFAQLYRSLMYSAQKTMDLPFLEASTLRAGERPELCRVSLPEFQQFLLDYQGELWAVDRLQVQEFMLSFLRDPLREIEEPYFFLDEFVTFLFSKENSVWNSQLDAVCPDTMNNPLSHYWISSSHNTYLTGDQFSSESSLEAYARCLRMGCRCIELDCWDGPDGMPVIYHGHTLTTKIKFSDVLHTIKEHAFVASEYPVILSIEDHCSIAQQRNMAQYFKKVLGDTLLTKPVEISADGLPSPNQLKRKILIKHKKLAEGSAYEEVPTSMMYSENDISNSIKNGILYLEDPVNHEWYPHYFVLTSSKIYYSEETSSDQGNEDEEEPKEVSSSTELHSNEKWFHGKLGAGRDGRHIAERLLTEYCIETGAPDGSFLVRESETFVGDYTLSFWRNGKVQHCRIHSRQDAGTPKFFLTDNLVFDSLYDLITHYQQVPLRCNEFEMRLSEPVPQTNAHESKEWYHASLTRAQAEHMLMRVPRDGAFLVRKRNEPNSYAISFRAEGKIKHCRVQQEGQTVMLGNSEFDSLVDLISYYEKHPLYRKMKLRYPINEEALEKIGTAEPDYGALYEGRNPGFYVEANPMPTFKCAVKALFDYKAQREDELTFIKSAIIQNVEKQEGGWWRGDYGGKKQLWFPSNYVEEMVNPVALEPEREHLDENSPLGDLLRGVLDVPACQIAIRPEGKNNRLFVFSISMASVAHWSLDVAADSQEELQDWVKKIREVAQTADARLTEGKIMERRKKIALELSELVVYCRPVPFDEEKIGTERACYRDMSSFPETKAEKYVNKAKGKKFLQYNRLQLSRIYPKGQRLDSSNYDPLPMWICGSQLVALNFQTPDKPMQMNQALFMTGRHCGYVLQPSTMRDEAFDPFDKSSLRGLEPCAISIEVLGARHLPKNGRGIVCPFVEIEVAGAEYDSTKQKTEFVVDNGLNPVWPAKPFHFQISNPEFAFLRFVVYEEDMFSDQNFLAQATFPVKGLKTGYRAVPLKNNYSEDLELASLLIKIDIFPAKENGDLSPFSGTSLRERGSDASGQLFHGRAREGSFESRYQQPFEDFRISQEHLADHFDSRERRAPRRTRVNGDNRL

SEQ ID NO: 51 - DNA, 1857 bp, Artificial sequence
  Synthetic polynucleotide
atgcctgatcctgctgctcatctgccattcttctacggcagcatctctagagccgaggccgaggaacatctgaaactggccggaatggccgatggcctgttcctgctgagacagtgtctgagatctctcggcggctacgtgctgtctctggtgcacgatgtgcggttccatcacttccccatcgagagacagctgaacggcacatatgccattgccggcggaaaagcccattgtggacctgccgaactgtgcgagttctacagcagagatcctgacggcctgccttgcaacctgagaaagccctgcaacagacctagcggccttgaacctcagcctggcgtgttcgattgcctgagagatgctatggtccgagactacgtgcggcagacatggaaactggaaggcgaggccctggaacaggcaatcatttctcaggcccctcaggtggaaaagctgatcgccacaacagcccacgagcggatgccttggtatcacagcagcctgacaagagaggaagccgagagaaagctgtactctggcgcccagaccgacggcaaatttctgctgaggcctcggaaagagcagggcacatacgccctgagcctgatctacggcaagaccgtgtaccactacctgatcagccaggacaaggccggcaagtactgtatccctgagggcaccaagttcgacaccctgtggcagctggtggaatacctgaagctgaaggccgacggactgatctactgcctgaaagaggcctgtcctaacagcagcgcctctaatgctagcggagctgctgctcctacactgcctgctcacccttctacactgacacaccctcagcggcggatcgatacactgaactctgatggctacacccctgagccagccagaatcacaagccccgacaagcccagacctatgcctatggataccagcgtgtacgagagcccctacagcgaccctgaggaactgaaggacaagaagctgttcctgaagcgggacaacctgctgattgccgacatcgaactcggctgcggcaattttggctctgtcagacagggcgtgtaccggatgcggaagaaacagatcgacgtggccatcaaggtgctgaagcagggaaccgagaaggccgataccgaggaaatgatgcgcgaggcccagatcatgcaccagctggacaatccttacatcgtgcggctgatcggcgtgtgtcaagccgaagctctgatgctggttatggaaatggcaggcggcggacccctgcacaagtttctcgttggcaagagagaggaaatccccgtcagcaacgtggccgaactgctgcaccaagtgtctatgggcatgaagtacctggaagagaagaacttcgtgcaccgcgacctggccgccagaaatgtgctgctggtcaacagacactacgccaagatcagcgacttcggcctgtctaaagccctgggcgccgacgatagctactacacagccagatctgccggaaagtggcccctgaagtggtacgcccctgagtgcatcaacttcagaaagttcagcagccgcagcgacgtgtggtcttacggcgttacaatgtgggaagccctgagctacggccagaaaccttacaagaagatgaagggccccgaagtcatggccttcatcgaacagggcaagagaatggaatgccctcctgagtgccctccagagctgtatgccctgatgagcgactgctggatctataagtgggaagatcggcccgacttcctgaccgtggaacagagaatgagagcctgctactacagcctggcctccaaggttgaaggccctcctggatctacacagaaggctgaagctgcctgcgct

SEQ ID NO: 52 - DNA, 2760 bp, Artificial sequence
  Synthetic polynucleotide
atgcctgatcctgctgctcatctgccattcttctacggcagcatctctagagccgaggccgaggaacatctgaaactggccggaatggccgatggcctgttcctgctgagacagtgtctgagatctctcggcggctacgtgctgtctctggtgcacgatgtgcggttccatcacttccccatcgagagacagctgaacggcacatatgccattgccggcggaaaagcccattgtggacctgccgaactgtgcgagttctacagcagagatcctgacggcctgccttgcaacctgagaaagccctgcaacagacctagcggccttgaacctcagcctggcgtgttcgattgcctgagagatgctatggtccgagactacgtgcggcagacatggaaactggaaggcgaggccctggaacaggcaatcatttctcaggcccctcaggtggaaaagctgatcgccacaacagcccacgagcggatgccttggtatcacagcagcctgacaagagaggaagccgagagaaagctgtactctggcgcccagaccgacggcaaatttctgctgaggcctcggaaagagcagggcacatacgccctgagcctgatctacggcaagaccgtgtaccactacctgatcagccaggacaaggccggcaagtactgtatccctgagggcaccaagttcgacaccctgtggcagctggtggaatacctgaagctgaaggccgacggactgatctactgcctgaaagaggcctgtcctaacagcagcgcctctaatgctagcggagctgctgctcctacactgcctgctcacccttctacactgacacaccctcagcggcggatcgatacactgaactctgatggctacacccctgagccagccagaatcacaagccccgacaagcccagacctatgcctatggataccagcgtgtacgagagcccctacagcgaccctgaggaactgaaggacaagaagctgttcctgaagcgggacaacctgctgattgccgacatcgaactcggctgcggcaattttggctctgtcagacagggcgtgtaccggatgcggaagaaacagatcgacgtggccatcaaggtgctgaagcagggaaccgagaaggccgataccgaggaaatgatgcgcgaggcccagatcatgcaccagctggacaatccttacatcgtgcggctgatcggcgtgtgtcaagccgaagctctgatgctggttatggaaatggcaggcggcggacccctgcacaagtttctcgttggcaagagagaggaaatccccgtcagcaacgtggccgaactgctgcaccaagtgtctatgggcatgaagtacctggaagagaagaacttcgtgcaccgcgacctggccgccagaaatgtgctgctggtcaacagacactacgccaagatcagcgacttcggcctgtctaaagccctgggcgccgacgatagctactacacagccagatctgccggaaagtggcccctgaagtggtacgcccctgagtgcatcaacttcagaaagttcagcagccgcagcgacgtgtggtcttacggcgttacaatgtgggaagccctgagctacggccagaaaccttacaagaagatgaagggccccgaagtcatggccttcatcgaacagggcaagagaatggaatgccctcctgagtgccctccagagctgtatgccctgatgagcgactgctggatctataagtgggaagatcggcccgacttcctgaccgtggaacagagaatgagagcctgctactacagcctggcctccaaggttgaaggccctcctggatctacacagaaggctgaagctgcctgcgctcggaagcggaggggctccggagctaccaacttcagtctgctgaaacaggccggcgacgtggaagagaaccctggccctatgggggcaggtgccaccggccgcgcaatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttcaagaggtga

SEQ ID NO: 53 - DNA, 1056 bp, Artificial sequence
  Synthetic polynucleotide
ggatcccctaacagcagcgcctctaatgctagcggagctgctgctcctacactgcctgctcacccttctacactgacacaccctcagcggcggatcgatacactgaactctgatggctacacccctgagccagccagaatcacaagccccgacaagcccagacctatgcctatggataccagcgtgtacgagagcccctacagcgaccctgaggaactgaaggacaagaagctgttcctgaagcgggacaacctgctgattgccgacatcgaactcggctgcggcaattttggctctgtcagacagggcgtgtaccggatgcggaagaaacagatcgacgtggccatcaaggtgctgaagcagggaaccgagaaggccgataccgaggaaatgatgcgcgaggcccagatcatgcaccagctggacaatccttacatcgtgcggctgatcggcgtgtgtcaagccgaagctctgatgctggttatggaaatggcaggcggcggacccctgcacaagtttctcgttggcaagagagaggaaatccccgtcagcaacgtggccgaactgctgcaccaagtgtctatgggcatgaagtacctggaagagaagaacttcgtgcaccgcgacctggccgccagaaatgtgctgctggtcaacagacactacgccaagatcagcgacttcggcctgtctaaagccctgggcgccgacgatagctactacacagccagatctgccggaaagtggcccctgaagtggtacgcccctgagtgcatcaacttcagaaagttcagcagccgcagcgacgtgtggtcttacggcgttacaatgtgggaagccctgagctacggccagaaaccttacaagaagatgaagggccccgaagtcatggccttcatcgaacagggcaagagaatggaatgccctcctgagtgccctccagagctgtatgccctgatgagcgactgctggatctataagtgggaagatcggcccgacttcctgaccgtggaacagagaatgagagcctgctactacagcctggggaattcctga

SEQ ID NO: 54 - DNA, 1368 bp, Artificial sequence
  Synthetic polynucleotide
atggctcgctcggtgaccctggtctttctggtgcttgtctcactgaccggtttgtatgctgcttacactgatatcgaaatgaaccgcctgggtaaggccgcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaaggatcccctaacagcagcgcctctaatgctagcggagctgctgctcctacactgcctgctcacccttctacactgacacaccctcagcggcggatcgatacactgaactctgatggctacacccctgagccagccagaatcacaagccccgacaagcccagacctatgcctatggataccagcgtgtacgagagcccctacagcgaccctgaggaactgaaggacaagaagctgttcctgaagcgggacaacctgctgattgccgacatcgaactcggctgcggcaattttggctctgtcagacagggcgtgtaccggatgcggaagaaacagatcgacgtggccatcaaggtgctgaagcagggaaccgagaaggccgataccgaggaaatgatgcgcgaggcccagatcatgcaccagctggacaatccttacatcgtgcggctgatcggcgtgtgtcaagccgaagctctgatgctggttatggaaatggcaggcggcggacccctgcacaagtttctcgttggcaagagagaggaaatccccgtcagcaacgtggccgaactgctgcaccaagtgtctatgggcatgaagtacctggaagagaagaacttcgtgcaccgcgacctggccgccagaaatgtgctgctggtcaacagacactacgccaagatcagcgacttcggcctgtctaaagccctgggcgccgacgatagctactacacagccagatctgccggaaagtggcccctgaagtggtacgcccctgagtgcatcaacttcagaaagttcagcagccgcagcgacgtgtggtcttacggcgttacaatgtgggaagccctgagctacggccagaaaccttacaagaagatgaagggccccgaagtcatggccttcatcgaacagggcaagagaatggaatgccctcctgagtgccctccagagctgtatgccctgatgagcgactgctggatctataagtgggaagatcggcccgacttcctgaccgtggaacagagaatgagagcctgctactacagcctggggaattcctga

SEQ ID NO: 55 - DNA, 3870 bp, Artificial sequence
  Synthetic polynucleotide
atggctggcgctgcttctccttgtgctaatggatgtggacctggcgctccatctgatgccgaagtgctgcacctgtgcagatctctggaagtgggcaccgtgatgaccctgttctacagcaagaagtcccagcggcctgagagaaagaccttccaagtgaagctggaaacccggcagatcacctggtctagaggcgccgataagatcgagggcgccatcgacatcagagagatcaaagagatcagacccggcaagaccagccgggacttcgacagatatcaagaggaccccgccttcagacccgaccagagccactgttttgtgatcctgtacggcatggaatttcggctgaaaacactgagtctgcaggccaccagcgaggacgaagtgaacatgtggatcaagggcctgacctggctgatggaagataccctgcaagcccctacaccactgcagatcgagagatggctgcggaagcagttctactccgtggaccggaacagagaggacagaatcagcgccaaggacctgaagaacatgctgagccaagtgaactacagagtgcccaatatgcggttcctgagagagcggctgacagacctcgaacagagaagcggcgatatcacctacggccagttcgcccagctgtacagatccctgatgtacagcgcccagaaaacaatggacctgcctttcctggaagccagcacactgagagccggcgaaagacctgagctgtgtagagtgtccctgcctgagttccagcagttcctgctggattatcagggcgagctgtgggccgtcgatagactgcaggtccaagagtttatgctgagcttcctgcgggaccctctgagagagatcgaggaaccctacttcttcctggacgaatttgtgaccttcctgttcagcaaagagaacagcgtctggaacagccagctggacgccgtgtgtcccgacaccatgaacaatcctctgagccactactggatcagcagcagccacaatacctacctgaccggcgatcagttcagcagcgagagcagtctggaagcctacgccagatgcctgagaatgggctgcagatgcatcgagctggattgctgggatggccctgatggcatgcctgtgatctaccacggccacacactgaccaccaagatcaagttcagcgacgtgctgcacaccatcaaagaacacgccttcgtggccagcgagtaccccgtgattctgagcatcgaggaccactgctctatcgcccagcagaggaacatggcccagtacttcaagaaggtgctgggagacaccctgctgaccaagcctgtggaaatctctgccgatggcctgccttctccaaaccagctgaagcggaagatcctgatcaagcacaagaagctggccgagggcagcgcctatgaagaggtgccaaccagcatgatgtactccgagaacgacatcagcaacagcatcaagaacggcatcctgtacctggaagatcccgtgaaccacgagtggtatccccactacttcgtgctgaccagctccaagatctactacagcgaggaaaccagcagcgaccagggcaacgaggatgaagaggaacccaaagaggtgtccagcagcaccgagctgcacagcaatgagaagtggtttcacggcaagctcggcgctggcagagatggcagacatattgccgagagactgctgaccgagtactgcattgaaacaggcgcccctgacggcagctttctcgtgcgggaatctgagacattcgtgggcgactacaccctgagcttttggagaaacggcaaggtccagcactgcagaatccacagcagacaggacgccggcacacccaagttctttctgaccgacaacctggtgttcgacagcctgtacgacctgatcacccactaccagcaggtcccactgagatgcaacgagttcgagatgagactgagcgagcccgtgcctcagacaaacgcccacgagagcaaagaatggtatcacgccagcctgacaagagcccaggccgagcacatgctgatgagagttcctagagatggcgccttcctcgtgcggaagagaaacgagcctaacagctacgccatcagcttcagagccgagggaaagattaagcactgccgggtgcagcaagagggccagacagtgatgctgggcaacagcgagttcgattccctggtggatctgatcagctactacgagaagcaccctctctaccggaagatgaagctgagataccccatcaacgaggaagccctggaaaagatcggcaccgccgaacctgattacggcgccctgtatgagggcagaaaccccggcttctacgtggaagctaaccccatgcctaccttcaagtgtgccgtgaaggccctgttcgactacaaggcccaaagagaggacgagctgaccttcatcaagagcgccatcattcagaacgtcgagaagcaagaaggcggctggtggcggggagattatggcggaaagaaacagctgtggttccccagcaactatgtggaagagatggtcaaccccgtggcactggaacccgagagagaacaccttgacgagaacagccctctgggcgatctgctgagaggtgttctggatgtgcctgcctgccagatcgccattagacccgagggcaagaacaaccggctgttcgtgttcagcatctctatggcctctgtggcccattggtcactggatgtggccgccgattctcaagaggaactgcaggactgggtcaagaaaatcagagaggtggcccagaccgccgatgccagactgacagagggcaaaatcatggaacggcggaagaagatcgccctggaactgtctgagctggtggtgtactgcagacccgtgcctttcgacgaagagaagattggcaccgagcgggcctgctacagagacatgagcagctttccagagacaaaggccgagaaatacgtgaacaaggccaagggaaagaagttcctgcagtataaccggctgcagctgagcagaatctaccccaagggccagagactggacagctccaactacgaccctctgcctatgtggatctgtggctctcagctggtggctctgaactttcagacccctgacaagcccatgcagatgaaccaggctctgttcatgaccggcagacactgcggatatgtgctgcagccctccaccatgagagatgaggccttcgatcccttcgacaagagcagcctgagaggcctggaaccttgtgccatcagcattgaggtcctgggcgccagacatctgcctaagaatggcagaggcatcgtgtgccccttcgtggaaattgaagtggctggcgccgagtacgacagcaccaagcagaaaaccgagttcgtggtggacaacggactgaaccctgtgtggcctgccaagccattccacttccaaatcagcaaccccgagttcgccttcctgagattcgtggtgtacgaagaggacatgttctccgaccagaacttcctggcacaggccacctttccagtgaagggactgaaaaccggctaccgggccgtgcctctgaaaaacaactactccgaggatctggaactggccagcctgctgatcaagatcgacatcttccccgccaaagaaaacggcgatctgagcccttttagcggcaccagcctcagagagagaggatctgacgcttctggccagctgttccacggtagagccagagagggcagcttcgagagcagataccagcagcctttcgaggacttccggatctctcaagagcacctggccgaccacttcgactctagagaaagaagggcccctcggagaaccagagtgaacggcgacaacagactc

SEQ ID NO: 56 - DNA, 4773 bp, Artificial sequence
  Synthetic polynucleotide
atggctggcgctgcttctccttgtgctaatggatgtggacctggcgctccatctgatgccgaagtgctgcacctgtgcagatctctggaagtgggcaccgtgatgaccctgttctacagcaagaagtcccagcggcctgagagaaagaccttccaagtgaagctggaaacccggcagatcacctggtctagaggcgccgataagatcgagggcgccatcgacatcagagagatcaaagagatcagacccggcaagaccagccgggacttcgacagatatcaagaggaccccgccttcagacccgaccagagccactgttttgtgatcctgtacggcatggaatttcggctgaaaacactgagtctgcaggccaccagcgaggacgaagtgaacatgtggatcaagggcctgacctggctgatggaagataccctgcaagcccctacaccactgcagatcgagagatggctgcggaagcagttctactccgtggaccggaacagagaggacagaatcagcgccaaggacctgaagaacatgctgagccaagtgaactacagagtgcccaatatgcggttcctgagagagcggctgacagacctcgaacagagaagcggcgatatcacctacggccagttcgcccagctgtacagatccctgatgtacagcgcccagaaaacaatggacctgcctttcctggaagccagcacactgagagccggcgaaagacctgagctgtgtagagtgtccctgcctgagttccagcagttcctgctggattatcagggcgagctgtgggccgtcgatagactgcaggtccaagagtttatgctgagcttcctgcgggaccctctgagagagatcgaggaaccctacttcttcctggacgaatttgtgaccttcctgttcagcaaagagaacagcgtctggaacagccagctggacgccgtgtgtcccgacaccatgaacaatcctctgagccactactggatcagcagcagccacaatacctacctgaccggcgatcagttcagcagcgagagcagtctggaagcctacgccagatgcctgagaatgggctgcagatgcatcgagctggattgctgggatggccctgatggcatgcctgtgatctaccacggccacacactgaccaccaagatcaagttcagcgacgtgctgcacaccatcaaagaacacgccttcgtggccagcgagtaccccgtgattctgagcatcgaggaccactgctctatcgcccagcagaggaacatggcccagtacttcaagaaggtgctgggagacaccctgctgaccaagcctgtggaaatctctgccgatggcctgccttctccaaaccagctgaagcggaagatcctgatcaagcacaagaagctggccgagggcagcgcctatgaagaggtgccaaccagcatgatgtactccgagaacgacatcagcaacagcatcaagaacggcatcctgtacctggaagatcccgtgaaccacgagtggtatccccactacttcgtgctgaccagctccaagatctactacagcgaggaaaccagcagcgaccagggcaacgaggatgaagaggaacccaaagaggtgtccagcagcaccgagctgcacagcaatgagaagtggtttcacggcaagctcggcgctggcagagatggcagacatattgccgagagactgctgaccgagtactgcattgaaacaggcgcccctgacggcagctttctcgtgcgggaatctgagacattcgtgggcgactacaccctgagcttttggagaaacggcaaggtccagcactgcagaatccacagcagacaggacgccggcacacccaagttctttctgaccgacaacctggtgttcgacagcctgtacgacctgatcacccactaccagcaggtcccactgagatgcaacgagttcgagatgagactgagcgagcccgtgcctcagacaaacgcccacgagagcaaagaatggtatcacgccagcctgacaagagcccaggccgagcacatgctgatgagagttcctagagatggcgccttcctcgtgcggaagagaaacgagcctaacagctacgccatcagcttcagagccgagggaaagattaagcactgccgggtgcagcaagagggccagacagtgatgctgggcaacagcgagttcgattccctggtggatctgatcagctactacgagaagcaccctctctaccggaagatgaagctgagataccccatcaacgaggaagccctggaaaagatcggcaccgccgaacctgattacggcgccctgtatgagggcagaaaccccggcttctacgtggaagctaaccccatgcctaccttcaagtgtgccgtgaaggccctgttcgactacaaggcccaaagagaggacgagctgaccttcatcaagagcgccatcattcagaacgtcgagaagcaagaaggcggctggtggcggggagattatggcggaaagaaacagctgtggttccccagcaactatgtggaagagatggtcaaccccgtggcactggaacccgagagagaacaccttgacgagaacagccctctgggcgatctgctgagaggtgttctggatgtgcctgcctgccagatcgccattagacccgagggcaagaacaaccggctgttcgtgttcagcatctctatggcctctgtggcccattggtcactggatgtggccgccgattctcaagaggaactgcaggactgggtcaagaaaatcagagaggtggcccagaccgccgatgccagactgacagagggcaaaatcatggaacggcggaagaagatcgccctggaactgtctgagctggtggtgtactgcagacccgtgcctttcgacgaagagaagattggcaccgagcgggcctgctacagagacatgagcagctttccagagacaaaggccgagaaatacgtgaacaaggccaagggaaagaagttcctgcagtataaccggctgcagctgagcagaatctaccccaagggccagagactggacagctccaactacgaccctctgcctatgtggatctgtggctctcagctggtggctctgaactttcagacccctgacaagcccatgcagatgaaccaggctctgttcatgaccggcagacactgcggatatgtgctgcagccctccaccatgagagatgaggccttcgatcccttcgacaagagcagcctgagaggcctggaaccttgtgccatcagcattgaggtcctgggcgccagacatctgcctaagaatggcagaggcatcgtgtgccccttcgtggaaattgaagtggctggcgccgagtacgacagcaccaagcagaaaaccgagttcgtggtggacaacggactgaaccctgtgtggcctgccaagccattccacttccaaatcagcaaccccgagttcgccttcctgagattcgtggtgtacgaagaggacatgttctccgaccagaacttcctggcacaggccacctttccagtgaagggactgaaaaccggctaccgggccgtgcctctgaaaaacaactactccgaggatctggaactggccagcctgctgatcaagatcgacatcttccccgccaaagaaaacggcgatctgagcccttttagcggcaccagcctcagagagagaggatctgacgcttctggccagctgttccacggtagagccagagagggcagcttcgagagcagataccagcagcctttcgaggacttccggatctctcaagagcacctggccgaccacttcgactctagagaaagaagggcccctcggagaaccagagtgaacggcgacaacagactccggaagcggaggggctccggagctaccaacttcagtctgctgaaacaggccggcgacgtggaagagaaccctggccctatgggggcaggtgccaccggccgcgcaatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttcaagaggtga

SEQ ID NO: 57 - DNA, 3891 bp, Artificial sequence
  Synthetic polynucleotide
ggatccggaggcggaggaagcgctggcgctgcttctccttgtgctaatggatgtggacctggcgctccatctgatgccgaagtgctgcacctgtgcagatctctggaagtgggcaccgtgatgaccctgttctacagcaagaagtcccagcggcctgagagaaagaccttccaagtgaagctggaaacccggcagatcacctggtctagaggcgccgataagatcgagggcgccatcgacatcagagagatcaaagagatcagacccggcaagaccagccgggacttcgacagatatcaagaggaccccgccttcagacccgaccagagccactgttttgtgatcctgtacggcatggaatttcggctgaaaacactgagtctgcaggccaccagcgaggacgaagtgaacatgtggatcaagggcctgacctggctgatggaagataccctgcaagcccctacaccactgcagatcgagagatggctgcggaagcagttctactccgtggaccggaacagagaggacagaatcagcgccaaggacctgaagaacatgctgagccaagtgaactacagagtgcccaatatgcggttcctgagagagcggctgacagacctcgaacagagaagcggcgatatcacctacggccagttcgcccagctgtacagatccctgatgtacagcgcccagaaaacaatggacctgcctttcctggaagccagcacactgagagccggcgaaagacctgagctgtgtagagtgtccctgcctgagttccagcagttcctgctggattatcagggcgagctgtgggccgtcgatagactgcaggtccaagagtttatgctgagcttcctgcgggaccctctgagagagatcgaggaaccctacttcttcctggacgaatttgtgaccttcctgttcagcaaagagaacagcgtctggaacagccagctggacgccgtgtgtcccgacaccatgaacaatcctctgagccactactggatcagcagcagccacaatacctacctgaccggcgatcagttcagcagcgagagcagtctggaagcctacgccagatgcctgagaatgggctgcagatgcatcgagctggattgctgggatggccctgatggcatgcctgtgatctaccacggccacacactgaccaccaagatcaagttcagcgacgtgctgcacaccatcaaagaacacgccttcgtggccagcgagtaccccgtgattctgagcatcgaggaccactgctctatcgcccagcagaggaacatggcccagtacttcaagaaggtgctgggagacaccctgctgaccaagcctgtggaaatctctgccgatggcctgccttctccaaaccagctgaagcggaagatcctgatcaagcacaagaagctggccgagggcagcgcctatgaagaggtgccaaccagcatgatgtactccgagaacgacatcagcaacagcatcaagaacggcatcctgtacctggaagatcccgtgaaccacgagtggtatccccactacttcgtgctgaccagctccaagatctactacagcgaggaaaccagcagcgaccagggcaacgaggatgaagaggaacccaaagaggtgtccagcagcaccgagctgcacagcaatgagaagtggtttcacggcaagctcggcgctggcagagatggcagacatattgccgagagactgctgaccgagtactgcattgaaacaggcgcccctgacggcagctttctcgtgcgggaatctgagacattcgtgggcgactacaccctgagcttttggagaaacggcaaggtccagcactgcagaatccacagcagacaggacgccggcacacccaagttctttctgaccgacaacctggtgttcgacagcctgtacgacctgatcacccactaccagcaggtcccactgagatgcaacgagttcgagatgagactgagcgagcccgtgcctcagacaaacgcccacgagagcaaagaatggtatcacgccagcctgacaagagcccaggccgagcacatgctgatgagagttcctagagatggcgccttcctcgtgcggaagagaaacgagcctaacagctacgccatcagcttcagagccgagggaaagattaagcactgccgggtgcagcaagagggccagacagtgatgctgggcaacagcgagttcgattccctggtggatctgatcagctactacgagaagcaccctctctaccggaagatgaagctgagataccccatcaacgaggaagccctggaaaagatcggcaccgccgaacctgattacggcgccctgtatgagggcagaaaccccggcttctacgtggaagctaaccccatgcctaccttcaagtgtgccgtgaaggccctgttcgactacaaggcccaaagagaggacgagctgaccttcatcaagagcgccatcattcagaacgtcgagaagcaagaaggcggctggtggcggggagattatggcggaaagaaacagctgtggttccccagcaactatgtggaagagatggtcaaccccgtggcactggaacccgagagagaacaccttgacgagaacagccctctgggcgatctgctgagaggtgttctggatgtgcctgcctgccagatcgccattagacccgagggcaagaacaaccggctgttcgtgttcagcatctctatggcctctgtggcccattggtcactggatgtggccgccgattctcaagaggaactgcaggactgggtcaagaaaatcagagaggtggcccagaccgccgatgccagactgacagagggcaaaatcatggaacggcggaagaagatcgccctggaactgtctgagctggtggtgtactgcagacccgtgcctttcgacgaagagaagattggcaccgagcgggcctgctacagagacatgagcagctttccagagacaaaggccgagaaatacgtgaacaaggccaagggaaagaagttcctgcagtataaccggctgcagctgagcagaatctaccccaagggccagagactggacagctccaactacgaccctctgcctatgtggatctgtggctctcagctggtggctctgaactttcagacccctgacaagcccatgcagatgaaccaggctctgttcatgaccggcagacactgcggatatgtgctgcagccctccaccatgagagatgaggccttcgatcccttcgacaagagcagcctgagaggcctggaaccttgtgccatcagcattgaggtcctgggcgccagacatctgcctaagaatggcagaggcatcgtgtgccccttcgtggaaattgaagtggctggcgccgagtacgacagcaccaagcagaaaaccgagttcgtggtggacaacggactgaaccctgtgtggcctgccaagccattccacttccaaatcagcaaccccgagttcgccttcctgagattcgtggtgtacgaagaggacatgttctccgaccagaacttcctggcacaggccacctttccagtgaagggactgaaaaccggctaccgggccgtgcctctgaaaaacaactactccgaggatctggaactggccagcctgctgatcaagatcgacatcttccccgccaaagaaaacggcgatctgagcccttttagcggcaccagcctcagagagagaggatctgacgcttctggccagctgttccacggtagagccagagagggcagcttcgagagcagataccagcagcctttcgaggacttccggatctctcaagagcacctggccgaccacttcgactctagagaaagaagggcccctcggagaaccagagtgaacggcgacaacagactctga

SEQ ID NO: 58 - DNA, 4203 bp, Artificial sequence
  Synthetic polynucleotide
atggctcgctcggtgaccctggtctttctggtgcttgtctcactgaccggtttgtatgctgcttacactgatatcgaaatgaaccgcctgggtaaggccgcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaaggatccggaggcggaggaagcgctggcgctgcttctccttgtgctaatggatgtggacctggcgctccatctgatgccgaagtgctgcacctgtgcagatctctggaagtgggcaccgtgatgaccctgttctacagcaagaagtcccagcggcctgagagaaagaccttccaagtgaagctggaaacccggcagatcacctggtctagaggcgccgataagatcgagggcgccatcgacatcagagagatcaaagagatcagacccggcaagaccagccgggacttcgacagatatcaagaggaccccgccttcagacccgaccagagccactgttttgtgatcctgtacggcatggaatttcggctgaaaacactgagtctgcaggccaccagcgaggacgaagtgaacatgtggatcaagggcctgacctggctgatggaagataccctgcaagcccctacaccactgcagatcgagagatggctgcggaagcagttctactccgtggaccggaacagagaggacagaatcagcgccaaggacctgaagaacatgctgagccaagtgaactacagagtgcccaatatgcggttcctgagagagcggctgacagacctcgaacagagaagcggcgatatcacctacggccagttcgcccagctgtacagatccctgatgtacagcgcccagaaaacaatggacctgcctttcctggaagccagcacactgagagccggcgaaagacctgagctgtgtagagtgtccctgcctgagttccagcagttcctgctggattatcagggcgagctgtgggccgtcgatagactgcaggtccaagagtttatgctgagcttcctgcgggaccctctgagagagatcgaggaaccctacttcttcctggacgaatttgtgaccttcctgttcagcaaagagaacagcgtctggaacagccagctggacgccgtgtgtcccgacaccatgaacaatcctctgagccactactggatcagcagcagccacaatacctacctgaccggcgatcagttcagcagcgagagcagtctggaagcctacgccagatgcctgagaatgggctgcagatgcatcgagctggattgctgggatggccctgatggcatgcctgtgatctaccacggccacacactgaccaccaagatcaagttcagcgacgtgctgcacaccatcaaagaacacgccttcgtggccagcgagtaccccgtgattctgagcatcgaggaccactgctctatcgcccagcagaggaacatggcccagtacttcaagaaggtgctgggagacaccctgctgaccaagcctgtggaaatctctgccgatggcctgccttctccaaaccagctgaagcggaagatcctgatcaagcacaagaagctggccgagggcagcgcctatgaagaggtgccaaccagcatgatgtactccgagaacgacatcagcaacagcatcaagaacggcatcctgtacctggaagatcccgtgaaccacgagtggtatccccactacttcgtgctgaccagctccaagatctactacagcgaggaaaccagcagcgaccagggcaacgaggatgaagaggaacccaaagaggtgtccagcagcaccgagctgcacagcaatgagaagtggtttcacggcaagctcggcgctggcagagatggcagacatattgccgagagactgctgaccgagtactgcattgaaacaggcgcccctgacggcagctttctcgtgcgggaatctgagacattcgtgggcgactacaccctgagcttttggagaaacggcaaggtccagcactgcagaatccacagcagacaggacgccggcacacccaagttctttctgaccgacaacctggtgttcgacagcctgtacgacctgatcacccactaccagcaggtcccactgagatgcaacgagttcgagatgagactgagcgagcccgtgcctcagacaaacgcccacgagagcaaagaatggtatcacgccagcctgacaagagcccaggccgagcacatgctgatgagagttcctagagatggcgccttcctcgtgcggaagagaaacgagcctaacagctacgccatcagcttcagagccgagggaaagattaagcactgccgggtgcagcaagagggccagacagtgatgctgggcaacagcgagttcgattccctggtggatctgatcagctactacgagaagcaccctctctaccggaagatgaagctgagataccccatcaacgaggaagccctggaaaagatcggcaccgccgaacctgattacggcgccctgtatgagggcagaaaccccggcttctacgtggaagctaaccccatgcctaccttcaagtgtgccgtgaaggccctgttcgactacaaggcccaaagagaggacgagctgaccttcatcaagagcgccatcattcagaacgtcgagaagcaagaaggcggctggtggcggggagattatggcggaaagaaacagctgtggttccccagcaactatgtggaagagatggtcaaccccgtggcactggaacccgagagagaacaccttgacgagaacagccctctgggcgatctgctgagaggtgttctggatgtgcctgcctgccagatcgccattagacccgagggcaagaacaaccggctgttcgtgttcagcatctctatggcctctgtggcccattggtcactggatgtggccgccgattctcaagaggaactgcaggactgggtcaagaaaatcagagaggtggcccagaccgccgatgccagactgacagagggcaaaatcatggaacggcggaagaagatcgccctggaactgtctgagctggtggtgtactgcagacccgtgcctttcgacgaagagaagattggcaccgagcgggcctgctacagagacatgagcagctttccagagacaaaggccgagaaatacgtgaacaaggccaagggaaagaagttcctgcagtataaccggctgcagctgagcagaatctaccccaagggccagagactggacagctccaactacgaccctctgcctatgtggatctgtggctctcagctggtggctctgaactttcagacccctgacaagcccatgcagatgaaccaggctctgttcatgaccggcagacactgcggatatgtgctgcagccctccaccatgagagatgaggccttcgatcccttcgacaagagcagcctgagaggcctggaaccttgtgccatcagcattgaggtcctgggcgccagacatctgcctaagaatggcagaggcatcgtgtgccccttcgtggaaattgaagtggctggcgccgagtacgacagcaccaagcagaaaaccgagttcgtggtggacaacggactgaaccctgtgtggcctgccaagccattccacttccaaatcagcaaccccgagttcgccttcctgagattcgtggtgtacgaagaggacatgttctccgaccagaacttcctggcacaggccacctttccagtgaagggactgaaaaccggctaccgggccgtgcctctgaaaaacaactactccgaggatctggaactggccagcctgctgatcaagatcgacatcttccccgccaaagaaaacggcgatctgagcccttttagcggcaccagcctcagagagagaggatctgacgcttctggccagctgttccacggtagagccagagagggcagcttcgagagcagataccagcagcctttcgaggacttccggatctctcaagagcacctggccgaccacttcgactctagagaaagaagggcccctcggagaaccagagtgaacggcgacaacagactctga

SEQ ID NO: 59 - protein, 542 aa, Artificial sequence
  Synthetic polypeptide
GSGGGGSALRNVPFRSEVLGWDPDSLADYFKKLNYRDCEKAVKKYHIDGARFLNLTENDIQKFPKLRVPILSKLSQEINKNEERRSIFTRKPQVPRFPEETESHEEDNGGWSSFEEDDYESPNDDQDGEDDGDYESPNEEEEAPVEDDADYEPPPSNDEEALQNSILPAKPFPNSNSMYIDRPPSGKTPQQPPVPPQRPMAALPPPPAGRNHSPLPPPQTNHEEPSRSRNHKTAKLPAPSIDRSTKPPLDRSLAPFDREPFTLGKKPPFSDKPSIPAGRSLGEHLPKIQKPPLPPTTERHERSSPLPGKKPPVPKHGWGPDRRENDEDDVHQRPLPQPALLPMSSNTFPSRSTKPSPMNPLPSSHMPGAFSESNSSFPQSASLPPYFSQGPSNRPPIRAEGRNFPLPLPNKPRPPSPAEEENSLNEEWYVSYITRPEAEAALRKINQDGTFLVRDSSKKTTTNPYVLMVLYKDKVYNIQIRYQKESQVYLLGTGLRGKEDFLSVSDIIDYFRKMPLLLIDGKNRGSRYQCTLTHAAGYPGNS

SEQ ID NO: 60 - protein, 646 aa, Artificial sequence
  Synthetic polypeptide
MARSVTLVFLVLVSLTGLYAAYTDIEMNRLGKAATTTPAPRPPTPAPTIASQPLSLRPEACRPAAGGAVHTRGLDFACDIYIWAPLAGTCGVLLLSLVITLYCKGSGGGGSALRNVPFRSEVLGWDPDSLADYFKKLNYRDCEKAVKKYHIDGARFLNLTENDIQKFPKLRVPILSKLSQEINKNEERRSIFTRKPQVPRFPEETESHEEDNGGWSSFEEDDYESPNDDQDGEDDGDYESPNEEEEAPVEDDADYEPPPSNDEEALQNSILPAKPFPNSNSMYIDRPPSGKTPQQPPVPPQRPMAALPPPPAGRNHSPLPPPQTNHEEPSRSRNHKTAKLPAPSIDRSTKPPLDRSLAPFDREPFTLGKKPPFSDKPSIPAGRSLGEHLPKIQKPPLPPTTERHERSSPLPGKKPPVPKHGWGPDRRENDEDDVHQRPLPQPALLPMSSNTFPSRSTKPSPMNPLPSSHMPGAFSESNSSFPQSASLPPYFSQGPSNRPPIRAEGRNFPLPLPNKPRPPSPAEEENSLNEEWYVSYITRPEAEAALRKINQDGTFLVRDSSKKTTTNPYVLMVLYKDKVYNIQIRYQKESQVYLLGTGLRGKEDFLSVSDIIDYFRKMPLLLIDGKNRGSRYQCTLTHAAGYPGNS

SEQ ID NO: 61 - DNA, 1629 bp, Artificial sequence
  Synthetic polynucleotide
ggatccggcggtggtggatctgctctgagaaacgtgcccttcagatccgaggttctcggctgggaccctgatagcctggccgactacttcaagaagctgaactacagggactgcgagaaggccgtgaagaagtaccatatcgacggcgccagattcctgaacctgaccgagaacgacatccagaagttccccaagctgagagtgcccatcctgagcaagctgagccaagagatcaacaagaacgaggaacggcggagcatcttcaccagaaagccccaggtgccaagattccccgaggaaacagagagccacgaggaagataacggcggctggtccagcttcgaagaggacgactacgagagccccaacgacgatcaggatggcgaggatgatggcgattacgagtcccctaacgaagaggaagaggcccctgttgaggacgacgccgattatgagcctcctcctagcaacgacgaagaagccctgcagaacagcatcctgcctgccaagccttttccaaacagcaacagcatgtacatcgaccggcctccaagcggcaaaacacctcagcaacctccagttcctcctcagcgacctatggctgcattgcctcctccaccagccggaagaaatcactctccactgcctccacctcagaccaatcacgaggaacccagcagaagcagaaaccacaagaccgccaagctgcccgctccatccatcgatagaagcaccaagcctccactggacagatctctggcccctttcgatagagagcccttcacactgggcaagaagcctccattcagcgacaagcctagcatccctgctggaagaagcctgggcgagcatctgcctaagatccagaaacctcctctgccacctaccaccgagagacacgagagatctagccctctgcctggcaagaaaccacctgtgcctaaacacggctggggcccagacagaagggaaaacgacgaggacgatgtgcaccagaggccattgcctcaacctgctctgctgcccatgtccagcaacacattccccagcaggtccaccaaaccttctccaatgaatcccctgcctagcagccacatgcctggcgcctttagcgagagcaatagcagctttcctcagagcgccagcctgcctccttactttagccagggacctagcaaccggccacctatcagagccgagggcagaaattttcccctgccactgcctaacaagcccagacctccatctcctgccgaggaagagaacagcctgaacgaagagtggtacgtgtcctacatcaccagacctgaagccgaggctgccctgagaaagatcaaccaggatggcacctttctcgtgcgggacagcagcaaaaagaccaccaccaatccttacgtgctgatggtgctgtacaaggacaaggtgtacaacatccagatccgctaccagaaagaaagccaggtgtacctgctcggcaccggcctgagaggcaaagaggattttctgagcgtcagcgacatcatcgactactttagaaagatgcccctgctgctgatcgacggcaagaacagaggcagcagataccagtgcacactgacacacgctgccggctatcccgggaattcctga

SEQ ID NO: 62 - DNA, 1941 bp, Artificial sequence
  Synthetic polynucleotide
atggctcgctcggtgaccctggtctttctggtgcttgtctcactgaccggtttgtatgctgcttacactgatatcgaaatgaaccgcctgggtaaggccgcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaaggatccggcggtggtggatctgctctgagaaacgtgcccttcagatccgaggttctcggctgggaccctgatagcctggccgactacttcaagaagctgaactacagggactgcgagaaggccgtgaagaagtaccatatcgacggcgccagattcctgaacctgaccgagaacgacatccagaagttccccaagctgagagtgcccatcctgagcaagctgagccaagagatcaacaagaacgaggaacggcggagcatcttcaccagaaagccccaggtgccaagattccccgaggaaacagagagccacgaggaagataacggcggctggtccagcttcgaagaggacgactacgagagccccaacgacgatcaggatggcgaggatgatggcgattacgagtcccctaacgaagaggaagaggcccctgttgaggacgacgccgattatgagcctcctcctagcaacgacgaagaagccctgcagaacagcatcctgcctgccaagccttttccaaacagcaacagcatgtacatcgaccggcctccaagcggcaaaacacctcagcaacctccagttcctcctcagcgacctatggctgcattgcctcctccaccagccggaagaaatcactctccactgcctccacctcagaccaatcacgaggaacccagcagaagcagaaaccacaagaccgccaagctgcccgctccatccatcgatagaagcaccaagcctccactggacagatctctggcccctttcgatagagagcccttcacactgggcaagaagcctccattcagcgacaagcctagcatccctgctggaagaagcctgggcgagcatctgcctaagatccagaaacctcctctgccacctaccaccgagagacacgagagatctagccctctgcctggcaagaaaccacctgtgcctaaacacggctggggcccagacagaagggaaaacgacgaggacgatgtgcaccagaggccattgcctcaacctgctctgctgcccatgtccagcaacacattccccagcaggtccaccaaaccttctccaatgaatcccctgcctagcagccacatgcctggcgcctttagcgagagcaatagcagctttcctcagagcgccagcctgcctccttactttagccagggacctagcaaccggccacctatcagagccgagggcagaaattttcccctgccactgcctaacaagcccagacctccatctcctgccgaggaagagaacagcctgaacgaagagtggtacgtgtcctacatcaccagacctgaagccgaggctgccctgagaaagatcaaccaggatggcacctttctcgtgcgggacagcagcaaaaagaccaccaccaatccttacgtgctgatggtgctgtacaaggacaaggtgtacaacatccagatccgctaccagaaagaaagccaggtgtacctgctcggcaccggcctgagaggcaaagaggattttctgagcgtcagcgacatcatcgactactttagaaagatgcccctgctgctgatcgacggcaagaacagaggcagcagataccagtgcacactgacacacgctgccggctatcccgggaattcctga

(19) United States
(12) Patent Application Publication
(10) Pub. No.: US 2024/0173409 A1
(43) Pub. Date: May 30, 2024

(54) ENHANCING T CELL FUNCTION THROUGH THE USE OF PROXIMAL SIGNALING MOLECULES

(71) Applicant: The Board of Trustees of the Leland Stanford Junior University, Stanford, CA (US)

(72) Inventors: Robbie G. Majzner, Palo Alto, CA (US); Aidan Tousley, Stanford, CA (US); Maria Caterina Rotiroti, Stanford, CA (US)

(21) Appl. No.: 18/552,478

(22) PCT Filed: Mar. 30, 2022
(86) PCT No.: PCT/US22/22538

§ 371 Date: Sep. 26, 2023

Related U.S. Application Data
(60) Provisional application No. 63/168,624, filed on Mar. 31, 2021.

(51) Int. Cl. A61K 39/00; A61P 35/00; C07K 14/47; C12N 5/0783

(52) U.S. Cl. CPC A61K 39/4631; A61K 39/4611; A61K 39/4632; A61K 39/4644; A61P 35/00; C07K 14/47; C12N 5/0636; A61K 2239/21; A61K 2239/22; C07K 2319/03

(57) ABSTRACT

The present disclosure generally relates to, inter alia, an isolated recombinant cell overexpressing a SLP-76 polypeptide. The disclosure also provides compositions and methods useful for producing the isolated recombinant cells and the corresponding SLP-76 molecules, as well as methods for treatment of diseases, such as cancer, with such compositions.

### RELATED APPLICATIONS

This application claims priority to and benefit of U.S. Provisional Application No. 63/168,624, filed on Mar. 31, 2021; the contents of which are hereby incorporated in its entirety.

### INCORPORATION BY REFERENCE

This application contains a Sequence Listing, which is hereby incorporated herein by reference in its entirety. The accompanying Sequence Listing text file, named “078430-533001WOSequenceListing_ST25.txt,” was created on Mar. 30, 2022 and is 239,808 bytes.

### FIELD

The present disclosure relates generally to the fields of oncology and immuno-therapeutics, and particularly relates to compositions of polypeptides and polynucleotide encoding the polypeptides, e.g., SLP-76, that are capable of enhancing activation and functions of cells, such as T cells expressing chimeric antigen receptors (CARs) and/or T-cell receptors (TCR) in response to target antigens. The disclosure also provides compositions of recombinant cells containing the polypeptides and methods useful for producing such recombinant cells, as well as methods for enhancing T cell activity for treatment of conditions, such as diseases (e.g., cancer or autoimmune disease).

### BACKGROUND

Current T cell therapies, such as involving T cells expressing chimeric antigen receptors (CARs) and/or T-cell receptors (TCRs), have limits when the target antigen recognized by the CARs or TCRs is expressed at a low density. When tumor cells down-regulate the amount of tumor antigens expressed on their surface (usually designed as the target antigen for CARs or TCRs), current CARs or TCRs may be unable to activate T cells to inhibit or kill the tumor cells. In other scenarios, CARs or TCRs may disrupt the T cells even when the antigen density is normal. For example, some CARs or TCRs may have low affinities to their target antigens, or may lead to T cell exhaustion, thus attenuating or terminating the T cell therapeutic effects eventually. Consequently, there remains a need for modulating T cell activation to overcome these obstacles to enhance and/or extend the reach of these therapies.

### SUMMARY

The present disclosure relates generally to the development of immuno-therapeutics, including recombinant cells or lysates of the recombinant cells, containing polypeptides, or polynucleotides encoding such polypeptides, such as lymphocyte cytosolic protein 2 (SH2 domain containing leukocyte protein of 76 kDa), also known as LCP2 or SLP-76, either in a cytosolic form or a membrane-bound form, as well as pharmaceutical compositions containing the same for use in enhancing immune therapies in, e.g., treating various conditions, such as diseases (e.g., cancer). As described in greater detail below, different SLP-76 constructs have been prepared and found to have dramatic effects to enhance activation and increase potency of immune cells (e.g., T cells) expressing CARs and/or TCRs in response to antigens, even when the antigen is expressed at a low density. In some embodiments, the SLP-76 construct is bound to the membrane of T cells expressing CARs and/or TCRs.

In one aspect, the present disclosure provides a composition containing an isolated recombinant cell modified to overexpress and/or contain elevated levels of a SLP-76 polypeptide. In some embodiments, the isolated recombinant cell of the composition described herein is capable of being activated by a target antigen expressed at a low density.

In some embodiments, the isolated recombinant cell further contains a T-cell receptor (TCR) polypeptide, a chimeric antigen receptor (CAR) polypeptide, and/or another receptor polypeptide. In some embodiments, the TCR or the CAR polypeptide has a binding affinity for the target antigen or for an adaptor molecule specifically recognizing the target antigen. In some embodiments, the isolated recombinant cell contains a TCR polypeptide. In some embodiments, the target antigen for the TCR polypeptide is expressed by a target cell and presented to the TCR polypeptide by an antigen-presenting cell (APC). In some embodiments, the isolated recombinant cell comprises an endogenous or native TCR polypeptide. In some embodiments, the isolated recombinant cell comprises a CAR polypeptide. In some embodiments, the target antigen for the CAR polypeptide is expressed by a target cell.

In some embodiments, the target cell described herein is a cell correlated to a condition, including, e.g., a proliferative disease (such as a cancer or a tumor), a hematological malignancy, a solid tumor, an autoimmune disease, an inflammation, an allergic disease, an infection, and/or a senescence/aging. In some embodiments, the target cell is a cancer cell.

In some embodiments, the CAR polypeptide described herein contains
a) an extracellular ligand-binding domain having a binding affinity for the target antigen or for an adaptor molecule specifically recognizing the target antigen;
b) a transmembrane domain;
c) an intracellular signaling domain comprising a proximal signaling molecule; and
d) optionally, a hinge domain and/or a costimulatory domain.

In some embodiments, the intracellular signaling domain of the CAR polypeptide described herein contains a full-length or biologically active fragment of a protein kinase, a G protein, a GTP-binding protein, an adaptor signaling protein, or a scaffold protein capable of inducing host cell activation. In some embodiments, the intracellular signaling domain of the CAR polypeptide contains CD3ζ, CD3-epsilon, CD3-gamma, DAP12, ZAP70, PLCG1, PKC, ITK, NCK, VAV1, GRB2, GADS, SOS1, ADAP, SYK, LYN, PI3K, or BLNK, or a biologically active fragment, mutant, or variant thereof.

In some embodiments, the TCR polypeptide and/or the CAR polypeptide induce exhaustion of the recombinant cell. In some embodiments, overexpression of the SLP-76 polypeptide described herein in the isolated recombinant cell does not enhance exhaustion of the recombinant cell. In some embodiments, overexpression of the SLP-76 polypeptide described herein in the isolated recombinant cell overcomes exhaustion of the recombinant cell.

In some embodiments, the isolated recombinant cell further expresses a normal density of the TCR polypeptide and/or the CAR polypeptide. In some embodiments, the isolated recombinant cell further expresses a high density of the TCR polypeptide and/or the CAR polypeptide. In some embodiments, the isolated recombinant cell further expresses a low density of the TCR polypeptide and/or the CAR polypeptide.

In some embodiments, the SLP-76 polypeptide is in a free cytosolic form. In some embodiments, the SLP-76 polypeptide is bound to the recombinant cell membrane. In some embodiments, the SLP-76 polypeptide is bound to the cell membrane via a transmembrane domain. In some embodiments, the SLP-76 polypeptide is bound to the cell membrane via:
i) an interaction between the SLP-76 polypeptide and a membrane protein;
ii) a covalent bond between the SLP-76 polypeptide and a fatty acid in the membrane; and/or
iii) a binding between the SLP-76 polypeptide and a lipid polar head group in the membrane.

In some embodiments, the SLP-76 polypeptide contains a full-length SLP-76. In some embodiments, the SLP-76 polypeptide contains a biologically active fragment, mutant, or variant of SLP-76. In some embodiments, the SLP-76 polypeptide contains at least one mutation (e.g., a K30R substitution) to a wild-type SLP-76 polypeptide. In some embodiments, the SLP-76 polypeptide contains an amino acid sequence having at least 30%, 40%, 50%, 60%, 70%, 75%, 80%, 85%, 90%, 91%, 92%, 93%, 94%, 95%, 96%, 97%, 98%, 99%, or more identity to SEQ ID NO: 1, 7, 14, 16, 18, 19, 40, 59 or 60. In some embodiments, the SLP-76 polypeptide contains an amino acid sequence having at least 70% or more identity to SEQ ID NO: 1, 7, 14, 16, 18, 19, 40, 59 or 60. In some embodiments, the SLP-76 polypeptide described herein contains the amino acid sequence of SEQ ID NO: 1, 7, 14, 16, 18, 19, 40, 59 or 60. In some embodiments, the SLP-76 polypeptide described herein consists of the amino acid sequence of SEQ ID NO: 1, 7, 14, 16, 18, 19, 40, 59 or 60.

In some embodiments, the isolated recombinant cell is an immune cell. In some embodiments, the isolated recombinant cell is a non-immune cell. In some embodiments, the isolated recombinant cell is a T cell, a regulatory T cell (Treg), a tumor-infiltrating lymphocyte (TIL), a natural killer (NK) cell, a macrophage, a monocyte, a gamma delta T cell, a stem cell, a natural killer T (NKT) cell, an induced pluripotent stem cell (iPSC)-derived NK cell, or an induced pluripotent stem cell (iPSC)-derived T cell.

In some embodiments, the isolated recombinant cell is capable of being activated by less than about 10, 100, 200, 300, 400, 500, 1000, 2000, 3000, 4000, 5000, 6000, 7000, 8000, 9000, or 10000, 20000, 30000, 40000, 50000, 60000, 70000, 80000, 90000, 100000, 200000, 300000, 400000, 500000, 600000, 700000, 800000, 900000, 1 million, 2 million, 3 million, 4 million, 5 million, 6 million, 7 million, 8 million, 9 million, or 10 million molecules of the target antigen.

In some embodiments, activation of the isolated recombinant cell in response to the target antigen enhances cell proliferation, differentiation, cytokine production and/or cytotoxicity.

In another aspect, the present disclosure provides an isolated polypeptide containing a SLP-76 polypeptide containing an amino acid sequence having at least 30%, 40%, 50%, 60%, 70%, 75%, 80%, 85%, 90%, 91%, 92%, 93%, 94%, 95%, 96%, 97%, 98%, 99%, or more identity sequence identity to SEQ ID NO: 1, 7, 14, 16, 18, 19, 40, 59 or 60. In some embodiments, the isolated polypeptide contains a SLP-76 polypeptide containing an amino acid sequence having at least 70%, 75%, 90%, 95%, or more identity sequence identity to SEQ ID NO: 1, 7, 14, 16, 18, 19, 40, 59 or 60. In some embodiments, the isolated polypeptide contains a SLP-76 polypeptide as described herein and a transmembrane domain. In some non-limiting embodiments, the transmembrane domain of the isolated polypeptide tethers the isolated polypeptide to a membrane (e.g., the cell membrane) of the isolated recombinant cell.

In another aspect, the present disclosure provides an isolated polynucleotide encoding an isolated SLP-76 polypeptide described herein. In some embodiments, the isolated polynucleotide encodes an isolated membrane-bound SLP-76 polypeptide described herein. Such SLP-76 polypeptide may be bound to the membrane of the isolated recombinant cell through a transmembrane domain or other mechanisms described herein.

In another aspect, the present disclosure provides an expression vector containing an isolated polynucleotide described herein. In some embodiments, the expression vector contains an expression promoter. In some embodiments, the isolated polynucleotide is conjugated to an expression promoter to be expressed in the isolated recombinant cell described herein.

In another aspect, the present disclosure provides a host cell comprising an expression vector described herein. In some embodiments, the host cell is a recombinant cell described herein, including, e.g., an immune cell or a non-immune cell.

In another aspect, the present disclosure provides a composition comprising
i) an isolated polynucleotide described herein and an isolated polynucleotide encoding a T-cell receptor (TCR) polypeptide and/or a chimeric antigen receptor (CAR) polypeptide;
ii) an expression vector described herein and an expression vector containing an isolated polynucleotide encoding a T-cell receptor (TCR) polypeptide and/or a chimeric antigen receptor (CAR) polypeptide; and/or
iii) an expression vector containing an isolated polynucleotide described herein and isolated polynucleotide encoding a T-cell receptor (TCR) polypeptide and/or a chimeric antigen receptor (CAR) polypeptide.
   In some embodiments, the composition is a pharmaceutical composition. In some embodiments, the pharmaceutical composition further contains a pharmaceutically acceptable carrier and/or excipient.

In another aspect, the present disclosure provides a method of producing a recombinant cell, including introducing a composition containing a polynucleotide encoding a SLP-76 polypeptide into a cell. In some embodiments, the cell is a native cell. In some embodiments, the cell is a recombinant cell. In some embodiments, the cell is an immune cell. In some embodiments, the cell is a non-immune cell. In some embodiments, the cell is derived from a biological sample. In some embodiments, the cell is derived from a biological sample from a human, such as a patient.

In some embodiments, the cell further comprises a TCR or CAR molecule. Such TCR molecule may be native or endogenous, or exogenous in the cell.

In another aspect, the present disclosure provides a method of producing a recombinant cell, including introducing a composition containing a polynucleotide encoding a SLP-76 polypeptide and a polynucleotide encoding a T-cell receptor (TCR) polypeptide and/or a chimeric antigen receptor (CAR) polypeptide into a cell. In some embodiments, the cell is a native cell. In some embodiments, the cell is a recombinant cell. In some embodiments, the cell is an immune cell. In some embodiments, the cell is a non-immune cell. In some embodiments, the cell is derived from a biological sample. In some embodiments, the cell is derived from a biological sample from a human, such as a patient.

In another aspect, the present disclosure provides a method of enhancing activation of a cell in response to a target antigen, including overexpressing a SLP-76 polypeptide in the cell. Such overexpression may be resulted from introducing a SLP-76 polypeptide, a polynucleotide encoding the SLP-76 polypeptide, and/or an expression vector containing the SLP-76 polynucleotide, as described herein, into the cell. In some embodiments, the cell is a native cell. In some embodiments, the cell is a recombinant cell. In some embodiments, the cell is an immune cell. In some embodiments, the cell is a non-immune cell. In some embodiments, the cell is derived from a biological sample. In some embodiments, the cell is derived from a biological sample from a human, such as a patient. In some embodiments, the cell further comprises a TCR or CAR molecule. Such TCR molecule may be native or endogenous, or exogenous in the cell.

In another aspect, the present disclosure provides a method of enhancing activation of a cell in response to a target antigen, including overexpressing a SLP-76 polypeptide and a T-cell receptor (TCR) polypeptide and/or a chimeric antigen receptor (CAR) polypeptide in the cell. In some embodiments, the cell is a native cell. In some embodiments, the cell is a recombinant cell. In some embodiments, the cell is an immune cell. In some embodiments, the cell is a non-immune cell. In some embodiments, the cell is derived from a biological sample. In some embodiments, the cell is derived from a biological sample from a human, such as a patient.

In another aspect, the present disclosure provides a method of treating a disease or disorder in a subject in need of, comprising administering to the subject a pharmaceutically effective amount of a composition described herein. Such composition may contain a SLP-76 polypeptide, a polynucleotide encoding the SLP-76 polypeptide, and/or an expression vector containing the SLP-76 polynucleotide, as described herein.

In some embodiments, a disease or disorder in a subject cannot be treated or can only be partly or less effectively treated by T cells expressing a T-cell receptor (TCR) polypeptide and/or a chimeric antigen receptor (CAR) polypeptide, while the TCR or the CAR polypeptide has a binding affinity to the target antigen or to an adaptor molecule specifically recognizing the target antigen expressed by a host cell related to the disease or disorder (e.g., a cancer or tumor cell). For example, the disease or disorder may involve a host cell expressing a low level (i.e., having a low density) of the target antigen, rendering an inefficient activation of the T cells through the TCR or the CAR polypeptide. Such low density of the target antigen may be caused by mutation(s) or transcriptional or translational regulation(s) in the host cell related to the disease or disorder. Such host cell may express a low density of the target antigen throughout the formation and development stages of the disease or disorder, or only after a certain timepoint in the formation and development stages of the disease or disorder. For example, some cancer or tumor cells may adopt a strategy of reducing expression of certain cancer/tumor-related antigens to escape the host immune system. In such scenario, a common CAR T or TCR therapy through recognizing the cancer/tumor-related antigens may not be effective or sufficient to treat the disease or disorder. Overexpressing SLP-76 (e.g., membrane-bound SLP-76 constructs) may enhance the activation and function of the CAR T or TCR-expressing T cells to treat the disease or disorder.

In another aspect, the present disclosure provides a method of treating a disease or disorder in a subject in need of, comprising administering to the subject a pharmaceutically effective amount of T cells expressing a CAR or TCR polypeptide specifically recognizing a target antigen expressed by a host cell related to the disease or disorder and further expressing a SLP-76 polypeptide (e.g., a membrane-bound SLP-76 polypeptide) described herein. Such host cell may express a low level (i.e., have a low density) of the target antigen, thus rendering similar T cells expressing only the CAR or TCR polypeptide but not expressing the SLP-76 polypeptide insufficient or incapable of treating the disease or disorder.

In another aspect, the present disclosure provides a method of treating a disease or disorder in a subject in need of, comprising administering to the subject a pharmaceutically effective amount of T cells expressing a CAR or TCR polypeptide specifically recognizing a target antigen expressed by a host cell related to the disease or disorder and further expressing a SLP-76 polypeptide (e.g., a membrane-bound SLP-76 polypeptide) described herein, while the disease or disorder may not be treated or may only be less effectively or insufficiently treated by similar T cells expressing only the CAR or TCR polypeptide but not expressing the SLP-76 polypeptide described herein. Such host cell may express a low level (i.e., have a low density) of the target antigen, thus rendering similar T cells expressing only the CAR or TCR polypeptide but not expressing the SLP-76 polypeptide described herein insufficient or incapable of treating the disease or disorder. In some embodiments, the method further contains a first step of detecting the expression levels of the target antigen(s) in the host cells related to the disease or disorder. In some embodiments, such expression levels of the target antigen(s) is known prior to the treatment.

In some embodiments, the method contains a first step of detecting the expression levels of the target antigen(s) in the host cells related to the disease or disorder in a subject in need of, and, if such expression levels are lower than a control level (e.g., the expression levels of the target antigen(s) in a healthy subject or a host cell not related to the disease or disorder), a second step of treating the subject with T cells expressing a CAR or TCR polypeptide specifically recognizing a target antigen expressed by a host cell related to the disease or disorder and further expressing a SLP-76 polypeptide (e.g., a membrane-bound SLP-76 polypeptide) described herein. In some embodiments, it is known prior to the treatment that a host cell related to the disease or disorder the in the subject expresses a low level of the target antigen(s) recognizable by the CAR or TCR molecule used in the CAR T or TCR therapy. In some embodiment, such low density of the target antigen(s) on the host cell results in a failure in treating the disease or disorder by the CAR T or TCR therapy alone. In some embodiment, such low density of the target antigen(s) on the host cell results in an ineffective or insufficient treatment of the disease or disorder by the CAR T or TCR therapy alone.

In some embodiments, the method further comprises an optional step, between the measurement/detection step and the treatment step described herein, to compare the measured/detected expression levels of the target antigen to a control level (e.g., the expression levels of the target antigen(s) in a healthy subject or a host cell not related to the disease or disorder). In some embodiments, the method further comprises an optional step, prior to the treatment step described herein, to overexpress a SLP-76 polypeptide (e.g., a membrane-bound SLP-76 polypeptide) described herein in the CAR T or TCR-expressing T cells. Such overexpression can be done by known methods (e.g., transduction, transfection, viral infection, etc.).

In another aspect, the present disclosure provides a method of treating a disease or disorder in a subject in need of, comprising
i) detecting the expression levels of a target antigen expressed by a host cell related to the disease or disorder in the subject, wherein the target antigen is recognizable by a T-cell receptor (TCR) polypeptide and/or a chimeric antigen receptor (CAR) polypeptide; and
ii) if the expression levels of the target antigen is lower than a control level, administering to the subject a pharmaceutically effective amount of T cells expressing the T-cell receptor (TCR) polypeptide and/or a chimeric antigen receptor (CAR) polypeptide and expressing a SLP-76 polypeptide.

In some embodiments, the method further comprises an optional step, prior to step ii), of comparing the expression levels of the target antigen by the host cell to a control level. In some embodiments, the method further comprises an optional step, prior to step ii), of overexpressing the SLP-76 polypeptide in T cells expressing the T-cell receptor (TCR) polypeptide and/or a chimeric antigen receptor (CAR) polypeptide.

In some embodiments, the SLP-76 polypeptide described herein is membrane-bound.

In some embodiments, the T cells expressing the T-cell receptor (TCR) polypeptide and/or a chimeric antigen receptor (CAR) polypeptide but not the SLP-76 polypeptide cannot treat or can only ineffectively or insufficiently treat the disease or disorder.

In another aspect, the present disclosure provides a method of treating a disease or disorder in a subject in need of, comprising administering to the subject a pharmaceutically effective amount of T cells (e.g., endogenous T cells in the subject) expressing a TCR polypeptide specifically recognizing a target antigen expressed by a host cell related to the disease or disorder and further expressing a SLP-76 polypeptide (e.g., a membrane-bound SLP-76 polypeptide) described herein, while the disease or disorder may not be treated or may only be less effectively or insufficiently treated by similar T cells expressing only the TCR polypeptide but not expressing the SLP-76 polypeptide described herein. Such host cell may express a low level (i.e., have a low density) of the target antigen, thus rendering the endogenous T cells expressing only the TCR polypeptide but not expressing the SLP-76 polypeptide described herein insufficient or incapable of treating the disease or disorder. In some embodiments, the method further contains a first step of detecting the expression levels of the target antigen(s) in the host cells related to the disease or disorder. In some embodiments, such expression levels of the target antigen(s) is known prior to the treatment. In some embodiments, the method further contains a step of isolating the T cells (e.g., endogenous T cells in the subject) from the subject. In some embodiments, the method further contains a step of expressing the SLP-76 polypeptide described herein in the isolated T cells (e.g., endogenous T cells). Such T cells may be any T cells expressing TCR molecules recognizing the target antigen, including, e.g., a regulatory T cell (Treg), a tumor-infiltrating lymphocyte (TIL), a natural killer (NK) cell, a macrophage, a monocyte, a gamma delta T cell, a stem cell, a natural killer T (NKT) cell, an induced pluripotent stem cell (iPSC)-derived NK cell, an induced pluripotent stem cell (iPSC)-derived T cell, or a mixture of T cells thereof. In some embodiments, the T cells (e.g., endogenous T cells in the subject) are tumor-infiltrating lymphocytes (TILs). In some embodiments, the T cells are isolated from the subject to be treated for the disease or disorder. In some embodiments, the T cells are isolated from a different subject, e.g., a different subject having the same disease or disorder, or a healthy subject. The SLP-76 polypeptide described herein may be introduced for expression in the isolated T cells (e.g., endogenous T cells in the subject) by any method described herein or known to a skilled artisan (e.g., through a viral vector).

In another aspect, the present disclosure provides a method of treating a disease or disorder in a subject in need of, comprising
i) detecting the expression levels of a target antigen expressed by a host cell related to the disease or disorder in the subject, wherein the target antigen is recognizable by a T-cell receptor (TCR) polypeptide expressed by a T cell in the subject;
ii) isolating the T cell in step i) from the subject; and
iii) if the expression levels of the target antigen in step i) is lower than a control level, expressing a SLP-76 polypeptide in the isolated T cell in step ii); and
   administering to the subject a pharmaceutically effective amount of the isolated T cells expressing the SLP-76 polypeptide.

In another aspect, the present disclosure provides a method of treating a disease or disorder in a subject in need of, comprising
i) isolating at least one T cell from the subject, wherein the at least one T cell expresses a T-cell receptor (TCR) polypeptide and/or a chimeric antigen receptor (CAR) polypeptide, wherein the TCR polypeptide and/or the CAR polypeptide specifically recognizes a target antigen expressed by a host cell related to the disease or disorder in the subject;
ii) expressing a SLP-76 polypeptide in the isolated at least one T cell; and
iii) administering to the subject a pharmaceutically effective amount of the isolated T cell expressing the SLP-76 polypeptide.

In some embodiments, the TCR polypeptide is endogenous to the subject. In some embodiments, the at least one T cell expressing the TCR polypeptide and/or the CAR polypeptide is endogenous to the subject. In some embodiments, the TCR polypeptide, the CAR polypeptide, and/or the at least one T cell expressing the TCR polypeptide and/or the CAR polypeptide is exogenous or genetically modified.

In some embodiments, the method further comprises a step, prior to step i), of measuring the expression level of the target antigen by the host cell.

In some embodiments, the method described herein further comprises an optional step, prior to step i), of comparing the expression level of the target antigen by the host cell to a control level.

In some embodiments, the levels of the target antigen expressed by the host cell is less than a control level. Such control level may refer to the expression levels of the target antigen by a different host cell not related to the disease or disorder (e.g., in the same or different tissue or organ in the same subject, or in the same or different tissue or organ in a different host either having the same disease or disorder or not having the disease or disorder, such as a healthy individual). Such control level may be already known to a doctor or physician prior to the treatment.

In some embodiments, the SLP-76 polypeptide described herein is introduced for expression in the isolated T cells by any method described herein or known to a skilled artisan (e.g., through a viral vector).

In some embodiments, the SLP-76 polypeptide is membrane-bound.

In some embodiments, the T cell in the subject is a regulatory T cell (Treg), a tumor-infiltrating lymphocyte (TIL), a natural killer (NK) cell, a macrophage, a monocyte, a gamma delta T cell, a stem cell, a natural killer T (NKT) cell, an induced pluripotent stem cell (iPSC)-derived NK cell, or an induced pluripotent stem cell (iPSC)-derived T cell. In some embodiments, the T cell in the subject is a tumor infiltrating lymphocyte (TIL).

In some embodiments, expressing the SLP-76 polypeptide enhances the activity of the at least one T cell to inhibit or kill the host cell.

In some embodiments, the host cell is a cancer or tumor cell.

Under a non-limiting mechanism, SLP-76 polypeptides described herein may enhance activity of another CAR or TCR molecule, either endogenous or exogenous, and/or reduce activation threshold of cells expressing the CAR or TCR molecule, in response to a target antigen, especially when the antigen is expressed at a low density. Expressing the SLP-76 polypeptides described herein may enhance the current T cell therapy using T cells expressing CAR or TCR molecules.

The SLP-76 polypeptide described herein may contain a full-length SLP-76 or a biologically active fragment, mutant, or variant of SLP-76. In some embodiments, the SLP-76 polypeptide contains at least one mutation to a wild-type SLP-76 polypeptide. Such mutation may be any of mutations known to a skilled artisan, such as a mutation to the K30 position (e.g., a K30R substitution) of a wild-type SLP-76. Under a non-limiting mechanism, any mutations not reducing SLP-76 expression level, stability and/or biological functions may be used herein. Some mutations may enhance SLP-76 expression level, stability and/or biological functions, such as a K30R substitution. SLP-76 mutants having such mutations may further enhance T cell activation upon binding to target antigen, and thus may further enhance treatment effects of CAR T or TCR therapies for diseases or disorders described herein.

### BRIEF DESCRIPTION OF THE DRAWINGS

FIG. 1 is a set of graphs showing T cell activity in response to tumor cells. Histograms of FACS analyses show the expression of a CD19-targeting CAR construct (“CD19-CD8TM-BBZ”, top left panel) or a HER2-targeting CAR construct (“HER-2-CD28TM-BBZ”, top right panel) in T cells, while the T cells further overexpress LCK (SEQ ID NO: 9, top trace), LAT (SEQ ID NO: 8, second trace from top), SLP-76 (SEQ ID NO: 7, third trace from top), or a control without proximal signaling molecules (bottom trace). T cells expressing different CAR constructs, with or without proximal signaling molecules, were incubated with control (“NO TUMOR”), wild-type NALM6 cells expressing CD19 but not HER2 (“WT NALM6”), or NALM6 cells expressing HER2 but not CD19 (“HER2+ NALM6”). The levels of IL-2 production by the T cells were measured and compared (bottom panel). CAR constructs used in each incubation condition are, from left to right, CD19-CD8TM-BBZ, CD19-CD8TM-BBZ+SLP-76, CD19-CD8TM-BBZ+LAT, CD19-CD8TM-BBZ+LCK, HER2-CD28TM-BBZ, HER2-CD28TM-BBZ+SLP-76, HER2-CD28TM-BBZ+LAT, and HER2-CD28TM-BBZ+LCK.

FIG. 2 is a bar graph showing IL-2 production levels of T cells in response to control (“ALONE”), wild-type NALM6 cells expressing a high level of CD19 (“WT N6”), or NALM6 cells expressing a low level of CD19 (“CD19 LOW N6”). The T cells further express the same CD19-targeting CAR construct (“CD19-CD8TM-BBZ”) as in FIG. 1, with or without SLP-76. CAR constructs used in each incubation condition are, from left to right, CD19-CD8TM-BBZ, and CD19-CD8TM-BBZ+SLP-76.

FIGS. 3A-3C are a set of graphs shows T cell activity in response to tumor cells. FIG. 3A shows FACS histograms detecting the expression of a CD19-targeting CAR construct (“CD19-CD8TM-BBZ”) on T cells, which further overexpress LCK (SEQ ID NO: 9; top trace), a membrane-bound LCK construct (SEQ ID NO: 17; second trace from top), SLP-76 (SEQ ID NO: 7; third trace from top), a membrane-bound SLP-76 (SEQ ID NO: 16; fourth trace from top), or control (bottom trace). FIG. 3B is a bar graph comparing IL-2 production levels of these T cells in response to control (“ALONE”), wild-type NALM6 cells (“WT N6”), or NALM6 cells expressing a low level of CD19 (“CD19 LOW N6”). CAR constructs used in each incubation condition are, from left to right, CD19-CD8TM-BBZ, CD19-CD8TM-BBZ+SLP-76, CD19-CD8TM-BBZ+membrane-bound SLP-76, CD19-CD8TM-BBZ+membrane-bound LCK, and CD19-CD8TM-BBZ+LCK. FIG. 3C is a graph comparing IL-2 production levels of T cells expressing the CD19-targeting CAR construct (“CD19-CD8TM-BBZ”) with or without the membrane-bound SLP-76. For experiment in FIG. 3C, an anti-idiotype antibody specifically recognizing the extracellular domain of the CD19-targeting CAR was coated to a plate in different amounts (the x-axis) by serial dilutions. T cells were incubated with each amount of the coated antibody and the corresponding production levels of IL-2 were measured to indicate the degrees of T cell activation.

FIGS. 4A-4C are a set of graphs shows T cell activity in response to tumor cells, similarly as in FIGS. 3A-3C. FIG. 4A shows FACS histograms detecting the expression of a HER2-targeting CAR construct (“HER-2-CD28TM-BBZ”) on T cells, which further overexpress LCK (SEQ ID NO: 9; top trace), a membrane-bound LCK construct (SEQ ID NO: 17; second trace from top), SLP-76 (SEQ ID NO: 7; third trace from top), a membrane-bound SLP-76 (SEQ ID NO: 16; fourth trace from top), or control (bottom trace). FIG. 4B is a bar graph comparing IL-2 production levels of these T cells in response to control (“NO TUMOR”), NALM6 cells expressing an ultra-low level of HER2 (“ULTRA LOW HER2+N6”), NALM6 cells expressing a low level of HER2 (“LOW HER2+N6”), NALM6 cells expressing a high level of HER2 (“HIGH HER2+N6”), or wild-type 143B cells (which express low levels of HER2). CAR constructs used in each incubation condition are, from left to right, HER2-CD28TM-BBZ, HER2-CD28TM-BBZ+SLP-76, HER2-CD28TM-BBZ+membrane-bound SLP-76, HER2-CD28TM-BBZ+LCK, and HER2-CD28TM-BBZ+membrane-bound LCK. FIG. 4C is a graph comparing IL-2 production levels of T cells expressing the same HER2-targeting CAR construct with or without overexpression of the membrane-bound SLP-76. Recombinant Her-2 antigen was coated to a plate in different amounts (the x-axis) by serial dilutions. T cells were incubated with each amount of the coated recombinant antigen and the corresponding production levels of IL-2 were measured to indicate the degree of T cell activation.

FIGS. 5A-5F are a set of graphs showing the capacity of a membrane-bound SLP-76 (SEQ ID NO: 16) to enhance CAR T cell activity in response to tumor cells. FIG. 5A shows FACS histograms detecting the expression of a CD19-targeting CAR construct (“CD19-CD28TM-CD28Z”, left panel) and a membrane-bound SLP-76 (through detecting a small extracellular tag conjugated to the membrane-bound SLP-76) (right panel) on T cells. FIG. 5B is a bar graph comparing IL-2 production levels of these T cells in response to control (“ALONE”), wild-type NALM6 cells (“WT N6”), or NALM 6 cells expressing a low level of CD19 (“CD19 LOW N6”). CAR constructs used in each incubation condition are, from left to right, mock, mock+membrane-bound SLP-76, CD19-CD28TM-CD28Z, and CD19-CD28TM-CD28Z+membrane-bound SLP-76. FIG. 5C is a graph comparing cytotoxicity of these T cells to CD19 LOW N6 cells overtime. Specifically, T cells expressing the CD19-targeting CAR construct, with or without a membrane-bound SLP-76, were incubated with CD19 LOW N6 cells expressing GFP and the number of the remaining N6 cells were counted after 3 hours to 72 hours of incubation based on their expression of GFP. The cytotoxicity index (% GFP signal normalized to the baseline measurement) was calculated to reflect the function of T cells to inhibit or kill the target cells. FIG. 5D shows the effect of the membrane-bound SLP-76 in an in vivo NALM6 stress test model. Specifically, T cells expressing the CD19-targeting CAR construct, with or without a membrane-bound SLP-76, were injected at sub-curative doses to animals having wild-type NALM-6 tumors. Tumor burden (shown in total flux number as they-axis) was measured and compared at different time points after treatment. FIG. 5E shows the survival of mice from the experiment described in FIG. 5D. The lines represent survival of mice expressing, from left to right, mock+membrane-bound SLP-76, CD19 28Z, or CD19 28Z+membrane-bound SLP-76. FIG. 5F shows the result in FIG. 5D (except for the mock+membrane SLP-76 treatment), after correction of a miscalculated data point.

FIGS. 6A-6F are a set of graphs showing the capacity of a membrane-bound SLP-76 to enhance CAR T cell activity in response to tumor cells. FIG. 6A shows FACS histograms detecting the expression of a CD22-targeting CAR construct (“CD22-CD8TM-BBZ”, left panel) and a membrane-bound SLP-76 (through detecting a small extracellular tag conjugated to the membrane-bound SLP-76) (right panel) on T cells. FIG. 6B is a bar graph comparing IL-2 production levels of these T cells, or mock controls, in response to control (“ALONE”), or NALM 6 cells expressing a low level of CD22 (“CD22LOW N6”). CAR constructs used in each incubation condition are, from left to right, mock, mock+membrane-bound SLP-76, CD22-CD8TM-BBZ, and CD22-CD8TM-BBZ+membrane-bound SLP-76. FIG. 6C is a graph comparing IL-2 production levels of T cells expressing the CD22-targeting CAR construct (“CD22-CD8TM-BBZ”) with or without the membrane-bound SLP-76. Recombinant CD22 was coated to a plate in different amounts (the x-axis) by serial dilutions. T cells were incubated with each amount of the coated CD22 and the production levels of TL-2 were measured to indicate T cell activation. FIGS. 6D-6F show the effect of the membrane-bound SLP-76 in an in vivo CD22LOW NALM6 (cells expressing a low density of CD22) model. Specifically, T cells expressing the CD22-targeting CAR construct, with or without overexpressing a membrane-bound SLP-76, were injected into animals having CD22LOW NALM-6 tumors. Tumor burden (shown in total flux number as the y-axis) was measured and compared at Day 22 after treatment (FIG. 6D). In a separate experiment, tumor burden (shown in total luminescence as the y-axis) was measured and compared at different time points after treatment (FIG. 6E). FIG. 6F shows the survival of mice from the experiment described in FIG. 6E.

FIGS. 7A-7D are a set of graphs showing the capacity of a membrane-bound SLP-76 to enhance CAR T cell activity in response to tumor cells. FIG. 7A shows FACS histograms detecting the expression of a B7-H3-targeting CAR construct (“B7-H3-CD28TM-BBZ”, left panel) and a membrane-bound SLP-76 (through detecting a small extracellular tag conjugated to the membrane-bound SLP-76) (right panel) on T cells. FIG. 7B is a graph comparing cytotoxicity of these CAR T cells to a neuroblastoma cell line CHLA-255 (which expresses a low density of B7-H3). Specifically, T cells expressing B7-H3-CD28TM-BBZ, with or without overexpressing a membrane-bound SLP-76, were incubated with CHLA-255 cells expressing GFP. The number of remaining CHLA-255 cells was counted after 3 hours to 78 hours of incubation, based on their expression levels of GFP. The cytotoxicity index was calculated to reflect the function of T cells to inhibit or kill the target cells. FIG. 7C shows the tumor burden in an in vivo CHLA-255 (which expresses a low density of B7-H3) metastatic model. T cells expressing B7-H3-CD28TM-BBZ, with or without overexpressing a membrane-bound SLP-76, were injected into animals having CHLA-255 tumors. Tumor burden (shown in total flux number as the y-axis) was measured at different time points after treatment. FIG. 7D compares animal survival after each treatment at different time points.

FIGS. 8A-8D are a set of graphs showing the capacity of a membrane-bound SLP-76 to enhance CAR T cell activity in response to tumor cells. FIG. 8A shows FACS histograms detecting the expression of a CD19-targeting CAR construct that contains a ZAP70²⁵⁵⁻⁶⁰⁰ fragment (containing the ZAP70 signaling domain) rather than a CD3-zeta signaling domain (“CD19-CD28TM-ZAP70”, left panel) and a membrane-bound SLP-76 (through detecting a small extracellular tag conjugated to the membrane-bound SLP-76) (right panel) on T cells. FIG. 8B compares IL-2 production levels of these T cells, or mock controls, in response to control (“NO TUMOR”), or wild-type NALM 6 cells. FIG. 8C shows the tumor burden in an in vivo NALM6 xenograft model. T cells expressing CD19-CD28TM-ZAP70, with or without overexpressing a membrane-bound SLP-76, were injected into animals having NALM6 tumors. Tumor burden (shown in total flux as they-axis) was measured and compared at different time points after treatment. FIG. 8D compares animal survival after each treatment at different time points.

FIGS. 9A-9B are a set of graphs showing the capacity of membrane-bound SLP-76 to enhance CAR T cell activity in response to tumor cells. FIG. 9A shows FACS histograms detecting the expression of a high affinity (HA) GD2-targeting CAR construct (“HA 28Z”, left panel) and two membrane-bound SLP-76 constructs, each containing a different hinge-transmembrane domain (CD8 vs. CD28) (through detecting an exemplary anti-HER2 ECD conjugated to the membrane-bound SLP-76 constructs) (right panel) on T cells. FIG. 9B compares IL-2 production levels (after baseline subtraction) of these T cells in response to NALM6 cells expressing GD2 (“GD2 N6”), wild-type 143B cells, or CHLA-255 cells. CAR constructs used in each incubation condition are, from left to right, HA-28Z, HA-28Z+CD8TM-SLP-76, and HA-28Z+CD28TM-SLP-76.

FIG. 10 is a set of graphs showing the expression of cell exhaustion biomarkers on CAR T cells. FACS analyses were performed to detect expression of TIM-3 (left panel), PD-1 (middle panel), and LAG-3 (right panel) on T cells expressing the B7-H3-CD28TM-BBZ CAR construct, with or without overexpression of a membrane-bound SLP-76.

FIG. 11 is a set of graphs showing the expression of cell exhaustion biomarkers on CAR T cells. FACS analyses were performed to detect expression of TIM-3 (left panel), PD-1 (middle panel), and LAG-3 (right panel) on T cells expressing the HA 28Z CAR construct, with or without overexpression of a membrane-bound SLP-76.

FIGS. 12A-12B are a set of graphs comparing the capacity of a membrane-bound SLP-76 vs. a LAT/SLP-76 chimera (LAT transmembrane fused to SLP-76) to enhance CAR T cell activity in response to tumor cells. FIG. 12A shows FACS histograms detecting the expression of the CD19 28Z CAR construct in T cells (left panel), which overexpress a membrane-bound SLP-76 construct (middle panel, right trace), or the LAT/SLP-76 chimera construct (right panel, right trace). In middle and right panels, the left trace represents controls without overexpression of either SLP-76 construct. FIG. 12B compares IL-2 production levels of these T cells in response to control (“ALONE”), wild-type NALM6 cells expressing a high antigen (i.e., CD19) density (“WT N6”), or either of two clones of NALM6 cells expressing low antigen density (“CLONE F N6” and “CLONE Z N6”). CAR constructs used in each incubation condition are, from left to right, mock, mock+LAT/SLP-76 chimera, mock+membrane-bound SLP-76, CD19-28Z, CD19-28Z+LAT/SLP-76 chimera, and CD19-28Z+membrane-bound SLP-76.

FIGS. 13A-13B are graphs showing the capacity of a membrane-bound SLP-76 (SEQ ID NO: 16) to enhance CAR T cell activity in response to tumor cells. FIG. 13A contains histograms of FACS analyses showing the expression of a CD19-targeting CAR construct (“CD19-CD8TM-BBZ”) in T cells. The T cells were further transduced to overexpress the membrane-bound SLP-76 construct (the top trace), a membrane-bound PLCG construct (the second trace from the top; SEQ ID NO: 50), a membrane-bound ZAP-70²⁵⁵⁻⁶⁰⁰ construct (the third trace from the top; SEQ ID NO: 46), a free PLCG construct (the fourth trace from the top; SEQ ID NO: 48), a free ZAP-70 construct (the fifth trace from the top; SEQ ID NO: 44), or mock control (the bottom trace). FIG. 13B contains a set of graphs showing cytokine (IL-2 in the top panel and IFNγ in the bottom panel) production by CD19-CD8TM-BBZ CAR T cells overexpressing, from left to right, mock control, ZAP-70, PLCg, membrane-bound ZAP-70²⁵⁵⁻⁶⁰⁰, membrane bound PLCg, or membrane bound SLP-76, when co-cultured with tumor cells (wild-type NALM-6).

FIGS. 14A-14E are graphs showing the capacity of a membrane-bound SLP-76 (SEQ ID NO: 16) to enhance CAR T cell activity in response to tumor cells expressing different levels of the antigen recognizable by the CAR molecule. FIG. 14A contains histograms of FACS analyses showing the expression of an Anaplastic Lymphoma Kinase (ALK)-targeting CAR construct (“ALK-CD8TM-BBZ”) in T cells. The T cells were further transduced to overexpress the membrane-bound SLP-76 construct (the top trace) or mock control (the second trace from the top). The bottom trace refers to control T cells not expressing the CAR construct or the membrane-bound SLP-76 construct. FIG. 14B contains histograms of FACS analyses showing the expression of the membrane-bound SLP-76 (by detecting its surface marker as described herein) in the T cells (the right trace). FIG. 14C contains histograms of FACS analyses showing expression levels of ALK in different Nalm-6 cells, including cells having a high level of ALK expression (ALK^{high}, the top trace), cells having a medium level of ALK expression (ALK^{med}, the second trace from the top), cells having a low level of ALK expression (ALK^{low}, the third trace from the top), or cells not expressing ALK (ALK⁻, the bottom trace). FIG. 14D contains graphs showing cytokine (IL-2 in the top panel, IFNγ in the bottom panel) production by ALK-CD8TM-BBZ CAR T cells with or without membrane bound SLP-76 overexpression, when co-cultured with tumor cells with different antigen densities. FIG. 14E contains graphs showing the killing of ALK Nalm-6 bearing different levels of ALK expression, by ALK-CD8TM-BBZ CAR T cells without (the top line in each panel) or with (the bottom line in each panel) membrane bound SLP-76 overexpression. The ratio of cell numbers between NALM-6 and T cells is listed on top of each panel.

FIGS. 15A-15C are graphs showing the capacity of a membrane-bound SLP-76 (SEQ ID NO: 16) to enhance T cell activity in response to tumor cells. FIG. 15A contains histograms of FACS analyses showing the expression of a NY-ESO-1 TCR construct (the left panel) or the membrane-bound SLP-76 construct (by detecting its surface marker as described herein; the right panel) in T cells. The tested T cells were transduced to overexpress the TCR construct and the membrane-bound SLP-76 construct (the top trace), the TCR construct only (the second trace from the top), a mock control and the membrane-bound SLP-76 construct (the third trace from the top), or the mock control only (the bottom trace). FIG. 15B contains a set of graphs showing IL-2 production by the NY-ESO-1 TCR T cells, when co-cultured with antigen positive tumor cells (A2+/NY-ESO-1+Nalm-6 cells, top panel, or A375 cells, bottom panel) or antigen negative tumor cells Mel888 (bottom panel). FIG. 15C illustrates the killing of A2+/NY-ESO-1+Nalm-6 cells by NY-ESO-1 TCR T cells with or without membrane bound SLP-76 overexpression, represented by the cytotoxicity index (they-axis) over the time periods post co-culturing (the x-axis, in hours).

FIGS. 16A-16C are graphs showing the capacity of a membrane-bound SLP-76 (SEQ ID NO: 16) to enhance T cell activity in response to tumor cells. FIG. 16A contains histograms of FACS analyses showing the expression of a B-cell maturation antigen (BCMA)-targeting CAR construct (the left panel) or the membrane-bound SLP-76 construct (by detecting its surface marker VSV-G as described herein; the right panel) in T cells. The tested T cells were transduced to overexpress the CAR construct and the membrane-bound SLP-76 construct (the top trace), the CAR construct only (the second trace from the top), or a mock control (the bottom trace). FIG. 16B contains a set of graphs comparing TL-2 (the top panel) or IFNγ (the bottom panel) production by the T cells expressing both the CAR construct and the membrane-bound SLP-76 construct or only the CAR construct, in response to MM1.S tumor cells. FIG. 16C compares the in vivo tumor control by these T cells, with or without membrane bound SLP-76 overexpression, in mice inoculated with luciferase-expressing MM1.S tumor cells and treated with the T cells.

FIGS. 17A-17B are graphs showing the capacity of a membrane-bound SLP-76 (SEQ ID NO: 16) to enhance T cell activity in response to tumor cells. FIG. 17A contains histograms of FACS analyses showing the expression of a B7-H3-targeting CAR construct (“B7-H3-CD28TM-BBZ”) in T cells. The tested T cells were transduced to overexpress the CAR construct and the membrane-bound SLP-76 construct (the top trace), the CAR construct only (the second trace from the top), or a mock control (the bottom trace). FIG. 17B compares the in vivo tumor control by these T cells with or without membrane bound SLP-76 overexpression in mice inoculated with luciferase-expressing diffuse intrinsic pontine glioma 6 xenografts (DIPG-6).

FIGS. 18A-18B are graphs showing the capacity of a membrane-bound SLP-76 (SEQ ID NO: 16) to enhance T cell proliferation in response to tumor cells. FIG. 18A contains histograms of FACS analyses showing the expression of a CD19-targeting CAR construct (“CD19-CD28TM-CD28Z”; the left panel) or the membrane-bound SLP-76 construct (by detecting its surface marker VSV-G as described herein; the right panel) in T cells. The tested T cells were transduced to overexpress both the CAR construct and the membrane-bound SLP-76 construct (the top trace) or only the CAR construct (the bottom trace). FIG. 18B compares the amount of CD19-CD28TM-CD28Z CAR T cells with or without membrane bound SLP-76 overexpression harvested from the spleens (top) or the bone marrow (bottom) of mice inoculated with luciferase-expressing WT Nalm-6 at different time points.

FIGS. 19A-19C are graphs showing the capacity of a membrane-bound SLP-76 (SEQ ID NO: 16) to enhance T cell proliferation in response to tumor cells. FIG. 19A contains histograms of FACS analyses showing the expression of a CD19-targeting CAR construct (“CD19-CD28TM-BBZ”; the left panel) or the membrane-bound SLP-76 construct (by detecting its surface marker VSV-G as described herein; the right panel) in T cells. The tested T cells were transduced to overexpress both the CAR construct and the membrane-bound SLP-76 construct (the top trace) or only the CAR construct (the bottom trace). FIG. 19B compares the in vivo tumor control by these T cells, with or without membrane bound SLP-76 overexpression, in mice inoculated with luciferase-expressing leukemia xenografts (WT Nalm-6). FIG. 19C compares the mice survival after each treatment through a time period.

FIGS. 20A-20C are graphs showing the capacity of a membrane-bound SLP-76 mutant (SEQ ID NO: 60) to enhance T cell activation in response to tumor cells. FIGS. 20A-20B contain histograms of FACS analyses showing the expression of a CD19-targeting CAR construct (“CD19-CD28TM-BBZ”; FIG. 20A) or various SLP-76 constructs (by detecting its surface marker VSV-G as described herein; FIG. 20B) in T cells. The tested T cells were transduced to overexpress both the CAR construct and a membrane-bound SLP-76 K30R mutant construct (the top trace), the membrane-bound SLP-76 (wild-type) construct (the middle trace), or only the CAR construct (the bottom trace). FIG. 20C compares the IL-2 expression levels by these T cells, when co-cultured with NALM-6 cells expressing low levels of CD19 or control (“NO TUMOR”). CAR constructs used in each incubation condition are, from left to right, the CD19BBZ CAR (i.e., CD19-CD28TM-BBZ) only, the CD19BBZ CAR+membrane-bound SLP-76, and the CD19BBZ CAR+membrane-bound SLP-76 K30R mutant.

### DETAILED DESCRIPTION OF THE DISCLOSURE

The present disclosure relates generally to, inter alia, compositions of polypeptides, e.g., SLP-76, and polynucleotide encoding the polypeptides, that are capable of enhancing activation and functions of cells [e.g., immune cells, such as T cells, expressing chimeric antigen receptors (CARs) and/or T-cell receptors (TCR)] in response to target antigens, such as to target, inhibit, and/or eliminate specific target cells (e.g., cancer or tumor cells) which express the target antigens. Furthermore, compositions of recombinant cells containing the polypeptides and methods useful for producing such recombinant cells, as well as methods for enhancing cell (e.g., immune cells) activity for treatment of conditions, such as diseases [e.g., proliferative diseases (such as cancers), hematological malignancies, solid tumors, autoimmune diseases, inflammations, allergic diseases, infections, senescence/aging, etc.], are also provided herein.

The exemplary experimental results presented herein demonstrate that SLP-76 were capable of enhancing cell (e.g., CAR T cells) activation, resulting in increased T cell effector functions and anti-tumor efficacy (e.g., increased cytokine production and cytotoxicity). Further, SLP-76, e.g., membrane-bound SLP-76, was shown to be capable of enhancing CAR T cell activity in response to a low antigen density, resulting in increased T cell effector functions and anti-tumor efficacy (e.g., enhanced cytokine production and cytotoxicity). Thus, SLP-76 may be capable of lowering T cell activation threshold, improving TCR or CAR function in immune cells, and improving immune cell functions by enhancing proximal signaling. Domain swaps have been used to engineer various CAR molecules or SLP-76 constructs to confirm that such capability of SLP-76 may be independent to CAR specificity or the structure of SLP-76 (e.g., not dependent on which transmembrane domain used to tether SLP-76 to the membrane). Other advantages for SLP-76, e.g., membrane-bound SLP-76, may include, e.g., not exacerbating cell exhaustion in immune cells (e.g., T cells), e.g., cells expressing CAR, TCR, or other molecules that cause tonic signaling. In particular, SLP-76 molecules may significantly enhance the activation of CAR T cells that otherwise display hallmarks of T cell exhaustion. While SLP-76, e.g., membrane-bound SLP-76, may promote CAR, TCR, or other receptor functions or other immune cell functions in most situations, it may have specific advantages when the target antigen is expressed at a low density, when a CAR, TCR, or another receptor construct has a low affinity to a target antigen, when a CAR, TCR or other receptor is expressed at a low density, and/or when a CAR, TCR, or another receptor construct induces cell exhaustion.

Nucleic acid molecules encoding these polypeptides and recombinant cells expressing these polypeptides are also provided. The disclosure also provides compositions and methods useful for producing such recombinant cells containing SLP-76 polypeptides, as well as methods for enhancing cell activation and preventing and/or treating conditions, such as proliferative diseases (e.g., cancers), hematological malignancies, solid tumors, autoimmune diseases, inflammations, allergic diseases, infections, senescence/aging, etc.

All publications and patent applications mentioned in this disclosure are herein incorporated by reference to the same extent as if each individual publication or patent application was specifically and individually indicated to be incorporated by reference.

### General Experimental Procedures

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference. As appropriate, procedures involving the use of commercially available kits and reagents are generally carried out in accordance with manufacturer defined protocols and/or parameters unless otherwise noted.

### Definition

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B”.

The term “about”, as used herein, has its ordinary meaning of approximately. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values.

The term “derived from” refers to the origin or source of a molecule, and may include naturally occurring, recombinant, unpurified, or purified molecules. Nucleic acid or polypeptide molecules are considered “derived from” when they include portions or elements assembled in such a way that they produce a functional unit. The portions or elements can be assembled from multiple sources provided that they retain evolutionarily conserved function. In some embodiments, the derivative nucleic acid or polypeptide molecules include substantially the same sequence as the source nucleic acid or polypeptide molecule. For example, the derivative nucleic acid or polypeptide molecules of the present disclosure may have at least 80%, 85%, 90%, 95%, 98%, 99%, or 100% sequence identity to the source nucleic acid or polypeptide molecule.

The term “recombinant” nucleic acid molecule, polypeptide, and/or cell, as used in the instant application, refers to a nucleic acid molecule, polypeptide, and/or cell that has been altered through human intervention. As non-limiting examples, a recombinant nucleic acid molecule can be one which: 1) has been synthesized or modified in vitro, for example, using chemical or enzymatic techniques, or recombination of nucleic acid molecules; 2) includes conjoined nucleotide sequences that are not conjoined in nature; 3) has been engineered using molecular cloning techniques such that it lacks one or more nucleotides with respect to the naturally occurring nucleic acid molecule sequence; and/or 4) has been manipulated using molecular cloning techniques such that it has one or more sequence changes or rearrangements with respect to the naturally occurring nucleic acid sequence. A non-limiting example of a recombinant protein is a SLP-76 polypeptide, a chimeric antigen receptor (CAR), a T-cell receptor (TCR), or other receptors as provided herein.

The terms “cell”, “cell culture”, “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the originally cell, cell culture, or cell line.

As used herein, a “host cell” refers to a cell for introduction of a nucleic acid and/or a polypeptide (e.g., SLP-76, CAR, and/or TCR molecules) described herein and/or a cell for expressing a nucleic acid or a polypeptide described herein. Host cells can be either untransformed cells or cells that have already been introduced with at least one nucleic acid molecule (e.g., SLP-76, CAR, and/or TCR molecules) described herein. A “recombinant cell” refers to a cell having genetic modifications and/or having introduced nucleic acids and/or polypeptides described herein.

As used herein, a “subject” or an “individual” includes animals, such as human (e.g., human subjects) and non-human animals. In some embodiments, a “subject” or “individual” is a patient under the care of a physician. Thus, the subject can be a human patient or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, and non-mammals, such as non-human primates, e.g., sheep, dogs, cows, chickens, amphibians, reptiles, etc.

The term “vector” is used herein to refer to a nucleic acid molecule or sequence capable of transferring or transporting another nucleic acid molecule. For example, a vector can be used as a gene delivery vehicle to transfer a gene into a cell. The transferred nucleic acid molecule is generally linked to, e.g., inserted into, the vector nucleic acid molecule. Generally, a vector is capable of replication when associated with the proper control elements. The term “vector” includes cloning vectors and expression vectors, as well as viral vectors and integrating vectors. An “expression vector” is a vector that includes a regulatory region, thereby capable of expressing DNA sequences and fragments in vitro and/or in vivo. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses. In some embodiments, a vector is a gene delivery vector.

It is understood that aspects and embodiments of the disclosure described herein include “comprising,” “consisting,” and “consisting essentially of” aspects and embodiments. As used herein, “comprising” is synonymous with “including”, “containing”, or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic characteristics of the claimed composition or method. Any recitation herein of the term “comprising”, particularly in a description of components of a composition or in a description of steps of a method, is understood to encompass those compositions and methods consisting essentially of and consisting of the recited components or steps.

Headings, e.g., (a), (b), (i) etc., are presented merely for ease of reading the specification and claims. The use of headings in the specification or claims does not require the steps or elements be performed in alphabetical or numerical order or the order in which they are presented.

As can be understood by one having ordinary skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As can also be understood by one skilled in the art all language such as “up to”, “at least”, “greater than”, “less than”, and the like include the number recited and refer to ranges which can be subsequently broken down into sub-ranges as discussed above. Finally, as can be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Compositions of the Disclosure

As described in greater detail below, the instant disclosure provides, inter alia, compositions of SLP-76 polypeptides, either bound to the cell membrane or not, capable of enhancing cell (e.g., an immune cell, such as T cells) activation. The cell activation may be induced by a natural/endogenous or recombinant T-cell receptor (TCR), a recombinant chimeric antigen receptor (CAR), or another natural/endogenous or recombinant receptor, which is expressed on the surface of the cell, specifically binding to a target antigen expressed by a target cell (e.g., a cancer or tumor cell). For example, a CAR may specifically bind to a target antigen expressed on the surface of a target cell, while a TCR may specifically bind to a target antigen (expressed by a target cell) presented by a major histocompatibility complex (MHC) on the surface of an antigen-presenting cell (APC). Furthermore, the SLP-76 polypeptides described herein may reduce T cell activation threshold by enhancing activation (e.g., through TCRs or CARs) in response to a low antigen density. In some embodiments, the instant disclosure provides compositions of the SLP-76 polypeptides, e.g., membrane-bound SLP-76 polypeptides, in combination with a recombinant CAR or a native or recombinant TCR. In some embodiments, the instant disclosure provides compositions of polynucleotides and expression vectors for expressing SLP-76 polypeptides, e.g., membrane-bound SLP-76 polypeptides. In some embodiments, the instant disclosure provides compositions of polynucleotides and expression vectors for expressing SLP-76 polypeptides, e.g., membrane-bound SLP-76 polypeptides, in combination with polynucleotides and expression vectors for expressing a recombinant CAR or a native or recombinant TCR. In some embodiments, the instant disclosure provides compositions of a recombinant cell containing polynucleotides and/or expression vectors for SLP-76. In some embodiments, the instant disclosure provides compositions of a recombinant cell containing polynucleotides and/or expression vectors for SLP-76, in combination with polynucleotides and/or expression vectors for a recombinant CAR or a native or recombinant TCR. In some embodiments, the instant disclosure provides a system containing a composition for SLP-76 described herein, alone or in combination with a composition for a CAR or TCR. In some embodiments, the instant disclosure provides a composition for SLP-76, alone or in combination with a composition for a CAR or TCR, for enhancing T cell activation in response to target antigens, especially to antigens expressed at a low density, for reducing T cell activation threshold, and/or for not exacerbating T cell exhaustion. In some embodiments, the instant disclosure provides a composition for SLP-76, alone or in combination with a composition for a CAR or TCR, for enhancing T cell activation in response to target antigens, especially when the CAR or TCR has low affinity to the target antigens. In some embodiments, the instant disclosure provides a composition for SLP-76, alone or in combination with a composition for a CAR or TCR, for preventing or treating a condition, such as a disease.

### SLP-76

The polypeptides of the present disclosure include a SLP-76 polypeptide, as described herein. SLP-76 was originally identified as a substrate of the ZAP-70 protein tyrosine kinase following T-cell receptor (TCR) ligation in the leukemic T cell line Jurkat. The SLP-76 locus has been localized to human chromosome 5q33 and the gene structure has been partially characterized in mice. The encoded SLP-76 protein associates with growth factor receptor bound protein 2, and is thought to play a role TCR-mediated intracellular signal transduction. A similar protein in mouse plays a role in normal T-cell development and activation. Mice lacking this gene show subcutaneous and intraperitoneal fetal hemorrhaging, dysfunctional platelets and impaired viability. Human and murine SLP-76 cDNAs both encode 533 amino acid proteins that are 72% identical and composed of three modular domains. The human SLP-76 protein can be accessed by its NCBI Reference Sequence No: NP_005556.1. The coding polynucleotide (e.g., mRNA) sequence can be accessed by NCBI Reference Sequence No: NM_005565.5. The NH₂-terminus of SLP-76 contains an acidic region that includes a PEST domain and several tyrosine residues that are phosphorylated following TCR ligation. SLP-76 also contains a central proline-rich domain and a COOH-terminal SH2 domain. A number of additional proteins have been identified that associate with SLP-76 both constitutively and inducibly following receptor ligation, supporting the notion that SLP-76 functions as an adaptor or scaffold protein. Studies using SLP-76-deficient T cell lines or mice have provided strong evidence that SLP-76 plays a positive role in promoting T cell development and activation as well as mast cell and platelet function. SLP-76 might serve as an integration point for signals by activating NK cell receptors. In NK cells, SLP-76 can be phosphorylated by SYK or ZAP70 following ligation of activating receptors.

The polypeptides in the present specification may include a full-length sequence of SLP-76, or a biologically functional fragment thereof, plus any mutants, variants, orthologues, fusions, or otherwise modified constructs. In some embodiments, the SLP-76 polypeptide described herein comprises at least one mutation (e.g., a mutation to the K30 position, such as a K30R substitution) to a wild-type SLP-76 polypeptide. For example, the SLP-76 polypeptide described herein may have an amino acid sequence having at least 30%, 40%, 50%, 60%, 70%, 75%, 80%, 85%, 90%, 91%, 92%, 93%, 94%, 95%, 96%, 97%, 98%, 99%, or more identity to SEQ ID NO: 1, 7, 14, 16, 18, 19, 40, 59 or 60. In some embodiments, the SLP-76 polypeptide described herein comprises the amino acid sequence of SEQ ID NO: 1, 7, 14, 16, 18, 19, 40, 59 or 60. In some embodiments, the SLP-76 polypeptide described herein consists of the amino acid sequence of SEQ ID NO: 1, 7, 14, 16, 18, 19, 40, 59 or 60.

The polypeptides in the present specification may include a SLP-76 construct in a free cytosolic form without binding to the cell membrane. Exemplary cytosolic forms of SLP-76 not bound to the membrane may include a full-length SLP-76 (e.g., SEQ ID NO: 1), a biologically active fragment, mutant, or variant of SLP-76, or a polypeptide containing a full-length, biologically active fragment, mutant, or variant of SLP-76 fused or conjugated to another molecule (e.g., a 2A-tNGFR polypeptide and/or a HA tag, having the sequence of any one of SEQ ID NOs: 4 to 6). In some embodiments, the SLP-76 polypeptide may be engineered to be bound to membrane. For example, SLP-76 may be fused or conjugated to another polypeptide or polynucleotide or other agents to be tethered to the membrane. In some embodiments, the SLP-76 polypeptides in the present specification may include a transmembrane domain conjugated to the full-length or fragments of SLP-76. Such transmembrane domain may be or derive from a transmembrane domain of a CAR molecule, a TCR molecule, or other transmembrane proteins. Exemplary transmembrane domains (TMDs) from CAR molecules may be found under the below section of descriptions for the CAR structure. Examples of suitable TMDs include, but are not limited to, a CD28 TMD, a CD8 TMD, a CD4 TMD, a CD3 TMD, a CTLA-4 TMD, an OX40 TMD, a 4-1BB TMD, a CD2 TMD, and a PD-1 TMD. Additional exemplary TMDs include TMDs from CD3D, CD3E, CD3G, CD3zeta, CD8a, CD8b, CD16, CD25, CD27, CD40, CD79A, CD79B, CD80, CD84, CD86, CD95, CD150 (SLAMFI), CD166, CD200R, CD223 (LAG3), CD270 (HVEM), CD272 (BTLA), CD273 (PD-L2), CD274 (PD-L1), CD278 (ICOS), CD300, CD357 (GITR), A2aR, ICAM-1, 2B4, BTLA, DAP10, FcRa, FcRP, Fyn, GAL9, IL7, IL12, IL15, KIR, KIR2DL4, KIR2DS1, LAG-3, Lck, LAT, LPA5, LRP, NKp30, NKp44, NKp46, NKG2C, NKG2D, NOTCH1, NOTCH2, NOTCH3, NOTCH4, PTCH2, ROR2, Ryk, SLP-76, SIRPa, pTa, T-cell receptor polypeptides (e.g., TCRa and TCRP), TIM3, TRIM, and ZAP70. Accordingly, in some embodiments, the TMD is derived from a CD28 TMD, a CD8 TMD, a CD4 TMD, a CD3 TMD, a CTLA4 TMD, an OX40 TMD, a 4-1BB TMD, a CD2 TMD, and a PD-1 TMD. In some embodiments, the TMD includes a CD28 TMD, a CD4 TMD, a CD8 TMD, a CD3 TMD, a CTLA4 TMD, an OX40 TMD, a 4-1BB TMD, a CD2 TMD, and a PD-1 TMD. In some embodiments, the SLP-76 polypeptides disclosed herein include a TMD derived from CD8. In some embodiments, the SLP-76 polypeptides disclosed herein include a CD8 TMD. In some embodiments, the SLP-76 polypeptides disclosed herein include a TMD derived from CD28. In some embodiments, the SLP-76 polypeptides disclosed herein include a CD28 TMD. In some embodiments, the SLP-76 polypeptides disclosed herein include a TMD containing an amino acid sequence having at least 30%, 40%, 50%, 60%, 70%, 75%, 80%, 85%, 90%, 91%, 92%, 93%, 94%, 95%, 96%, 97%, 98%, 99%, or more identity to SEQ ID NO: 12 or 13. In some embodiments, the SLP-76 polypeptides disclosed herein may further include an additional domain derived from a CAR or TCR molecule. For example, the SLP-76 polypeptides may include an extracellular domain or an extracellular tag further conjugated to a transmembrane domain. In some embodiments, the optional extracellular domain or tag in the SLP-76 polypeptides described herein may facilitate tethering SLP-76 to the cell membrane. In some embodiments, the optional extracellular domain or tag in the SLP-76 polypeptides described herein may provide a way of detecting the SLP-76 polypeptides (e.g., by FACS or other immunological methods). In some embodiments, the optional extracellular domain may be an ECD as described in details in below sections for CAR molecules. In some embodiments, the optional extracellular domain contains an antigen-binding domain. In some embodiments, the optional extracellular tag contains an amino acid sequence having at least 30%, 40%, 50%, 60%, 70%, 75%, 80%, 85%, 90%, 91%, 92%, 93%, 94%, 95%, 96%, 97%, 98%, 99%, or more identity to SEQ ID NO: 11 or 39. In some embodiments, the optional extracellular tag may be a molecule such as a polypeptide or a polynucleotide. In some embodiments, the optional extracellular tag contains a VSV-G polypeptide. In some embodiments, the optional extracellular tag contains an amino acid sequence having at least 30%, 40%, 50%, 60%, 70%, 75%, 80%, 85%, 90%, 91%, 92%, 93%, 94%, 95%, 96%, 97%, 98%, 99%, or more identity to SEQ ID NO: 10. Under a non-limiting mechanism, SLP-76 polypeptides described herein, with or without an optional extracellular domain or tag, do not recognize a target antigen and/or activate cells expressing the SLP-76 polypeptides by themselves. By contrast, they may enhance activity of another CAR or TCR molecule, either endogenous or exogenous, and/or reduce activation threshold of cells expressing the CAR or TCR molecule, in response to a target antigen, especially when the antigen is expressed at a low density.

In some embodiments, the SLP-76 polypeptides described herein may contain a protein domain or an adapter sequence conjugated to a full-length or fragment of SLP-76. In some embodiments, the protein domain or an adapter sequence may interact (e.g., through binding) with a membrane protein, thus tethering SLP-76 to the membrane.

In some embodiments, a full-length or fragment of SLP-76 may be tethered to the membrane through a covalent bond between SLP-76 and a fatty acid in the membrane. In some embodiments, a full-length or fragment of SLP-76 may be tethered to the membrane through binding to a lipid polar head group in the membrane.

In some embodiments, the SLP-76 polypeptides described herein may contain a membrane-binding domain conjugated to a full-length or fragment of SLP-76. In some embodiments, the membrane-binding domain may tether SLP-76 to the membrane. Exemplary membrane binding domain may include the C1, C2, PH, FYVE, PX, ENTH, and BAR domains, or other domains described in Hurley Biochim Biophys Acta. 2006; 1761:805-811, the content of which is incorporated herein by reference to its entirety.

In some embodiments, the SLP-76 polypeptides described herein may be cross-linked or chemically conjugated to any membrane protein or membrane fatty acid or lipid.

### Chimeric Antigen Receptors (Cars)

As described above, the SLP-76 polypeptides of the present disclosure are capable of enhancing activation of cells (e.g., immune cells, such as T cells, expressing CARs or TCRs) by target antigens. In principle, there are no particular limitations with regard to suitable CAR molecules for the enhancement function of SLP-76 described herein. In some embodiments, a CAR molecule described herein may include (i) an extracellular ligand-binding domain (a.k.a., extracellular antigen-binding domain, or ECD) having a binding affinity for a target antigen; (ii) a transmembrane domain (TMD); and (iii) an intracellular signaling domain (a.k.a., cytosolic signaling domain). In some embodiments, binding of the target antigen to the extracellular ligand-binding domain activates the intracellular signaling domain of a CAR polypeptide. In some embodiments, the disclosed CARs have the above listed domains in (i)-(iii) in a N-terminal to C-terminal direction. In some embodiments, the disclosed CARs are described in a format of X-Y-Z, wherein X represents the ligand/antigen recognizable by the extracellular ligand-binding domain, Y represents the hinge/transmembrane (H/TM) domain, and Z represents the intracellular signaling domain. For example, “CD19-CD28H/TM-CD28-CD3zeta,” “CD19-CD28TM-CD28Z,” or “CD19 28Z” refers to a CAR molecule having an extracellular ligand-binding domain which specifically binds to CD19, a CD28 hinge/transmembrane domain, a CD28 intracellular region, and a CD3zeta intracellular signaling domains. Unless specified otherwise, a “TM” domain in this application includes a TMD with or without an optional hinge domain.

In some embodiments, the disclosed CARs in cells further include one or more hinge domains and/or costimulatory domains.

In some embodiments, the disclosed CARs contain at least one intracellular (i.e., cytosolic) signaling domain described herein, including, but not limiting to, proximal signaling molecules, such as CD3zeta, ZAP70, PLCG1, PKC, ITK, NCK, VAV1, GRB2, GADS, SOS1, ADAP, SYK, LYN, PI3K, BLNK, or a biologically active fragment, mutant, or variant thereof. In some embodiments, the disclosed CARs contain more than one intracellular (i.e., cytosolic) signaling domain described herein, including, but not limiting to, proximal signaling molecules, such as CD3zeta, ZAP70, PLCG1, PKC, ITK, NCK, VAV1, GRB2, GADS, SOS1, ADAP, SYK, LYN, PI3K, BLNK, or a biologically active fragment, mutant, or variant thereof.

### Extracellular Ligand (Antigen)-Binding Domains (ECD)

A CAR molecule described herein has at least one ECD which has a binding affinity for one or more target ligands (or antigens, which are used interchangeably in the instant application). In some embodiments, the target antigen is expressed on a cell surface, or is otherwise anchored, immobilized, or restrained on a cell surface. Non-limiting examples of suitable target antigen types include cell surface receptors, adhesion proteins, carbohydrates, lipids, glycolipids, lipoproteins, and lipopolysaccharides that are surface-bound, integrins, mucins, and lectins. In some embodiments, the antigen is a protein. In some embodiments, the antigen is a carbohydrate. In some embodiments, the antigen is expressed by a target cell (e.g., a cancer/tumor cell). In some embodiments, the antigen is an adaptor molecule specifically recognizing a target cell (e.g., a cancer/tumor cell). In some embodiments, the antigen is a biomarker for a specific disease, disorder, or condition (e.g., a cancer/tumor). Non-limiting examples of suitable antigen include CD19, HER2, ROR1, B7-H3 (CD276), CD22, CD2, CD5, CD6, 4-1BB, GD2, FcγR1, and integrins, as well as those described in the below section titled “antigens”.

In some embodiments, the ECD of the CAR polypeptides disclosed herein includes an antigen-binding moiety that binds to one or more target antigens. In some embodiments, the antigen-binding moiety includes one or more antigen-binding determinants of an antibody or a functional antigen-binding fragment thereof, including, at least, a ligand-binding domain of an antibody, an antigen-binding fragment, an antibody mimetic, a receptor, or a ligand for a targeted receptor. One skilled in the art upon reading the present disclosure can readily understand that the term “functional fragment thereof” or “functional variant thereof” refers to a molecule having quantitative and/or qualitative biological activity in common with the wild-type molecule from which the fragment or variant was derived. For example, a functional fragment or a functional variant of an antibody is one which retains essentially the same ability to bind to the same epitope as the antibody from which the functional fragment or functional variant was derived. For instance, an antibody capable of binding to an epitope of a cell surface receptor may be truncated at the N-terminus and/or C-terminus, and the retention of its epitope binding activity assessed using assays known to those of skill in the art. In some embodiments, the antigen-binding moiety is selected from the group consisting of an antibody, a monoclonal antibody, an antigen-binding fragment (Fab), a nanobody, a diabody, a triabody, a minibody, an F(ab′)₂ fragment, an F(ab)v fragment, a single chain variable fragment (scFv), a single domain antibody (sdAb), a V_{H} domain, a V_{L} domain, an Fv fragment, a VNAR domain, and a V_{HH} domain, or a functional fragment thereof. In some embodiments, the antigen-binding moiety includes a heavy chain variable region and a light chain variable region. In some embodiments, the antigen-binding moiety includes a scFv. In some embodiments, the antibody mimetic is selected from the group consisting of: Affibody molecules, Affilins, Affimers, Alphabodies, Avimers, DARPins, Fynomers, Kunitz domain peptides, Monobodies, nanoCLAMPs, and a biologically active fragment thereof.

The antigen-binding moiety can include naturally-occurring amino acid sequences or can be engineered, designed, or modified so as to provide desired and/or improved properties, e.g., binding affinity. Generally, the binding affinity of an antibody or an antigen-binding moiety for a target antigen (e.g., CD19 antigen or HER2 antigen) can be calculated by the Scatchard method described by Frankel et al., Mol. Immunol, 16: 101-106, 1979. In some embodiments, binding affinity can be measured by an antigen/antibody dissociation rate. In some embodiments, a high binding affinity can be measured by a competition radioimmunoassay. In some embodiments, binding affinity can be measured by ELISA. In some embodiments, antibody affinity can be measured by flow cytometry. An antibody that “selectively binds” a target antigen (such as CD19 or HER2) is an antibody that binds the target antigen with high affinity and does not significantly bind other unrelated antigens but binds the antigen with high affinity, e.g., with an equilibrium constant (K_{D}) of 100 nM or less, such as 60 nM or less, for example, 30 nM or less, such as, 15 nM or less, or 10 nM or less, or 5 nM or less, or 1 nM or less, or 500 pM or less, or 400 pM or less, or 300 pM or less, or 200 pM or less, or 100 pM or less.

A skilled artisan can select an ECD based on the desired localization or function of a cell that is genetically modified to express a CAR polypeptide of the present disclosure. For example, a CAR polypeptide with an ECD including an antibody specific for a HER2 antigen can target cells to HER2-expressing breast cancer cells. In some embodiments, the ECD of the CAR polypeptides disclosed herein is capable of binding a tumor-associated antigen (TAA) or a tumor-specific antigen (TSA). A skilled artisan can understand that TAAs include a molecule, such as e.g., protein, present on tumor cells and on normal cells, or on many normal cells, but at much lower concentration than on tumor cells. In contrast, TSAs generally include a molecule, such as e.g., protein which is present on tumor cells but absent from normal cells.

### Antigens

In the instant application, the terms “ligand(s)” and “antigen(s)” are used interchangeably to mean a target molecule(s) specifically recognized by an extracellular antigen-binding domain of a CAR molecule described in this section or by a TCR molecule described in a following section. In principle, there are no particular limitations with regard to suitable target antigens. In some embodiments of the disclosure, the antigen-binding moiety of the ECD is specific for an epitope present in an antigen expressed or recognized by a target cell. In some embodiments, the target cell is correlated to a disease or disorder. Exemplary diseases or disorders may include, e.g., proliferative diseases (e.g., cancers), hematological malignancies, solid tumors, autoimmune diseases, inflammations, allergic diseases, infections, senescence/aging, etc. In some embodiments of the disclosure, the antigen-binding moiety of the ECD of a CAR is specific for an epitope present in an antigen that is expressed by a tumor cell, i.e., a tumor-associated antigen. The tumor-associated antigen can be an antigen associated with, e.g., a leukemia cell, a neuroblastoma cell, an osteosarcoma cell, a pancreatic cancer cell, a colon cancer cell, an ovarian cancer cell, a prostate cancer cell, a lung cancer cell, mesothelioma cell, a breast cancer cell, a urothelial cancer cell, a liver cancer cell, a head and neck cancer cell, a sarcoma cell, a cervical cancer cell, a stomach cancer cell, a gastric cancer cell, a melanoma cell, a uveal melanoma cell, a cholangiocarcinoma cell, a multiple myeloma cell, a lymphoma cell, a glioblastoma cell, or other cancer cells described in the present disclosure. In some embodiments, the antigen-binding moiety is specific for an epitope present in a tissue-specific antigen. In some embodiments, the antigen-binding moiety is specific for an epitope present in a disease-associated antigen. Tumors often refers to a subgroup of cancers when an uncontrolled growth of cells occurs in solid tissue such as an organ, muscle, or bone. In the instant application, the terms “tumors” and “cancers” are generally used interchangeably to mean cells having an uncontrolled growth, unless specified otherwise.

In some embodiments, the antigen is selected from the group consisting of CD19, HER2, ROR1, B7-H3 (CD276), CD22, GD2, CD2, CD5, CD6, 4-1BB, FcγR1, and integrins. In some embodiments, the antigen is selected from the group consisting of CD19, HER2, ROR1, B7-H3 (CD276), influenza hemagglutinin (HA), CD22, CD2, CD5, CD6, 4-1BB, FcγR1, GD2, CD22, CD10, CD20, GPC2, GD3, GM2, GM3, and integrins.

Non-limiting examples of suitable target antigens also include Glypican 2 (GPC2), human epidermal growth factor receptor 2 (Her2/neu), CD276 (B7-H3), PSMA, IL-13-receptor alpha 1, IL-13-receptor alpha 2, alpha-fetoprotein (AFP), carcinoembryonic antigen (CEA), cancer antigen-125 (CA-125), CA19-9, calretinin, MUC-1, epithelial membrane protein (EMA), epithelial tumor antigen (ETA). Other suitable target antigens include, but are not limited to, tyrosinase, melanoma-associated antigen (MAGE), CD34, CD45, CD123, CD93, CD99, CD117, chromogranin, cytokeratin, desmin, glial fibrillary acidic protein (GFAP), gross cystic disease fluid protein (GCDFP-15), ALK, DLK1, FAP, NY-ESO, WT1, HMB-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysin, thyroglobulin, thyroid transcription factor-1, CD138, FolR1, LeY, MCSP, and TYRP1.

Additional antigens that can be suitable for the CARs disclosed herein include, but are not limited to, the dimeric form of the pyruvate kinase isoenzyme type M2 (tumor M2-PK), CD19, CD20, CD5, CD7, CD3, TRBC1, TRBC2, BCMA, CD38, CD123, CD93, CD34, CDla, SLAMF7/CS1, FLT3, CD33, CD123, TALLA-1, CSPG4, DLL3, Kappa light chain, Lamba light chain, CD16/FcγRIII, CD64, FITC, CD22, CD27, CD30, CD70, GD2 (ganglioside G2), GD3, EGFRvIII (epidermal growth factor variant III), EGFR and isovariants thereof, TEM-8, sperm protein 17 (Sp17), and mesothelin. Further non-limiting examples of suitable antigens include PAP (prostatic acid phosphatase), prostate stem cell antigen (PSCA), prostein, NKG2D, TARP (T-cell receptor gamma alternate reading frame protein), Trp-p8, STEAPI (six-transmembrane epithelial antigen of the prostate 1), an abnormal ras protein, an abnormal p53 protein, integrin β3(CD61), galactin, K-Ras (V-Ki-ras2 Kirsten rat sarcoma viral oncogene), and Ral-B. In some embodiments, the antigen is CD19, human epidermal growth factor receptor 2 (Her2/neu), CD276 (B7-H3), or GD-2.

Antigens that can be suitable for the CARs disclosed herein include, but are not limited to, one, or any combination thereof, of: CD1a, CD1b, CD1c, CD2, CD3, CD4, CD5, CD6, CD7, CD8, CD9, CD10, CD11a, CD11b, CD11c, CD12, CD13, CD14, CD15 (SSEA-1), CD16 (FcγRIII), CD17, CD18, CD19, CD20, CD21, CD22, CD23, CD24, CD25, CD26, CD27, CD28, CD29, CD30, CD31, CD32 (FcγRII), CD33, CD34, CD35, CD36, CD37, CD38, CD39, CD40, CD41, CD43, CD44, CD44V6, CD45, CD45R/B220, CD45RO, CD49b, CD49d, CD49f, CD52, CD53, CD54, CD56 (NCAM), CD57, CD61 (integrin 03), CD62L, CD63, CD64, CD66b, CD68, CD69, CD70, CD73, CD74, CD79a (Iga), CD79b (Igo), CD80, CD83, CD85k (ILT3), CD86, CD88, CD93 (ClRqp), CD94, CD95, CD99, CD103, CD105 (Endoglin), CD107a, CD107b, CD114 (G-CSFR), CD115, CD117, CD122, CD123, CD129, CD133, CD134, CD138 (Syndecan-1), CD141, CD146, CD152 (CTLA-4), CD158 (Kir), CD161 (NK-1.1), CD163, CD183, CD191, CD193 (CCR3), CD194 (CCR4), CD195 (CCR5), CD197 (CCR7), CD203c, CD205 (DEC-205), CD207 (Langerin), CD209 (DC-SIGN), CD223, CD235, CD244 (2B4), CD252 (OX40L), CD267, CD268 (BAFF-R), CD273 (B7-DC, PD-L2), CD276 (B7-H3), CD279 (PD1), CD282 (TLR2), CD284 (TLR4), CD294, CD304 (Neuropilin-1), CD305, CD314 (NKG2D), CD319 (CRACC), CD326, CD328 (Siglec-7), CD335 (NKp46), fetal acetylcholine receptor (AChR), ADGRE2, alpha-fetoprotein (AFP), ALK, BCMA, BDCA3, C3AR, Lewis A (CA19.9), carbonic anhydrase IX (CA1X), calretinin, cancer antigen-125 (CA-125), CCR1, CCR4, CDS, carcinoembryonic antigen (CEA), chromogranin, CLEC12A, an antigen of a cytomegalovirus (CMV) infected cell (e.g., a cell surface antigen), CS-1, CSPG4, cytokeratin, desmin, DLK1, DLL3, EGFRvIII (epidermal growth factor variant III), EGFR and isovariants thereof, epithelial cell adhesion molecule (EpCAM), epithelial glycoprotein2 (EGP 2), epithelial glycoprotein-40 (EGP-40), epithelial membrane protein (EMA), ERBB, epithelial tumor antigen (ETA), FAP, folate-binding protein (FBP), FcγR1, FcεRIα, FITC, FLT3, FOLR1, FOLR3, galactin, ganlgiosides, gross cystic disease fluid protein (GCDFP-15), GD2 (ganglioside G2), GD3, GM2, GM3, glial fibrillary acidic protein (GFAP), gpA33, glycopeptides, Glypican 2 (GPC2), oncofetal antigen (h5T4), influenza hemagglutinin (HA), human epidermal growth factor receptor 2 (Her2/neu), HLA-DR, HM1.24, HMB-45 antigen, HPV E6, HPV E7, ICAM-1, IgG, IgD, IgE, IgM, IL-13-receptor alpha 1, integrins, Integrin B7, Interleukin-13 receptor subunit alpha-2 (IL-13Rα2), Kappa light chain, kinase insert domain receptor (KDR), Lamba light chain, LILRB2, Lewis Y (LeY), LGR5, Ly49, Ly108, L1 cell adhesion molecule (L1-CAM), melanoma-associated antigen (MAGE), melanoma antigen family A 1 (MAGE-A1), protein melan-A (melanoma antigen recognized by T lymphocytes; MART-1), MCSP, c-Met, MICA/B, mesothelin, muscle-specific actin (MSA), Mesothelin (MSLN), the dimeric form of the pyruvate kinase isoenzyme type M2 (tumor M2-PK), Mucin 1 (Muc-1), Mucin 16 (Muc-16), myo-D1, Necl-2, neurofilament, NKCSI, NKG2D, neuron-specific enolase (NSE), NY-ESO, cancer-testis antigen NY-ESO-1, an abnormal p53 protein, PAP (prostatic acid phosphatase), PAMA, P-cadherin, placental alkaline phosphatase, PRAIVIE, prostein, prostate stem cell antigen (PSCA), prostate-specific membrane antigen (PSMA), Ral-B, K-Ras (V-Ki-ras2 Kirsten rat sarcoma viral oncogene), an abnormal ras protein, ROR1, SLAMF7/CS1, receptor tyrosine-protein kinases erb-B2,3,4, sperm protein 17 (Sp17), STEAPI (six-transmembrane epithelial antigen of the prostate 1), synaptophysin, tumor-associated glycoprotein 72 (TAG-72), TALLA-1, TARP (T-cell receptor gamma alternate reading frame protein), TEM-8, human telomerase reverse transcriptase (hTERT), TIM-3, TLR4, TRBC1, TRBC2, Trp-p8, thyroglobulin, thyroid transcription factor-1, TYRP1, tyrosinase, vascular endothelial growth factor R2 (VEGF-R2), Vα24, Wilms tumor protein (WT-1), and various pathogen antigen known in the art. In some embodiments, an antigen described herein is a tumor-specific antigen (TSA) or a tumor-associated antigen (TAA).

In some embodiments, the CAR polypeptides disclosed herein include an ECD including an antigen-binding moiety that binds CD19. In some embodiments, the CAR polypeptides disclosed herein include an ECD including an antigen-binding moiety that binds HER2. In some embodiments, the CAR polypeptides disclosed herein include an ECD including an antigen-binding moiety that binds B7-H3. In some embodiments, the CAR polypeptides disclosed herein include an ECD including an antigen-binding moiety that binds GD2. In some embodiments, the CAR polypeptides disclosed herein include an ECD including an antigen-binding moiety that binds CD22. In some embodiments, the CAR polypeptides disclosed herein include an ECD including an antigen-binding moiety having an amino acid sequence exhibiting at least 50% sequence identity to any ECD described herein. In some embodiments, the antigen-binding moiety has an amino acid sequence exhibiting at least 50%, at least 60%, at least 70%, at least 80%, at least 85%, at least 90%, at least 95%, at least 96%, at least 97%, at least 98%, at least 99%, or 100% sequence identity to any ECD described herein. The percent identity as used herein refers to two or more sequences or subsequences that are the same or have a specified percentage of nucleotides or amino acids that are the same over a specified region. The two or more sequences or subsequences may be compared and aligned for maximum correspondence over a comparison window or designated region, as measured by, e.g., a BLAST or BLAST 2.0 sequence comparison algorithms, or by manual alignment and visual inspection. See e.g., the NCBI web site at ncbi.nlm.nih.gov/BLAST. This definition also refers to, or may be applied to, the complement of a sequence. This definition also includes sequences that have deletions and/or additions, as well as those that have substitutions. Sequence identity can be calculated using published techniques and widely available computer programs, such as the GCS program package (Devereux et al, Nucleic Acids Res. 12:387, 1984), BLASTP, BLASTN, FASTA (Atschul et al., J Mol Biol 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof. The amino acid substitution(s) may be a conservative amino acid substitution, for example at a non-essential amino acid residue in the CDR sequence(s). A “conservative amino acid substitution” is understood to be one in which the original amino acid residue is substituted with an amino acid residue having a similar side chain. Families of amino acid residues having similar side chains are known in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

One advantage of SLP-76 polypeptides described herein is that they are capable of enhancing cell activation in response to target antigens. In some embodiments, such activation is induced by CAR or TCR molecules expressed on the surface of the cell specifically binding to the target antigens. Further, SLP-76 polypeptides, e.g., membrane-bound SLP-76 polypeptides described herein, are capable of enhancing cell activation when the target antigens are expressed at a low density, and/or reducing cell activation threshold through CAR or TCR molecules. The term “a low density of antigens”, unless specified elsewhere in the instant disclosure, generally refers to a situation when, in a microenvironment containing at least one cell expressing CAR or TCR molecules and a target antigen (e.g., a tumor antigen expressed by a tumor cell, or a tumor antigen presented by an antigen-presenting cell), the amount of the target antigen molecules per cell is not enough to activate (through binding) a sufficient number of the CAR or TCR molecules required for activating the cell expressing the CAR or TCR molecules. The density of a target antigen is a relative term, dependent on the antigen, the CAR or TCR structures, and/or the target cell expressing or presenting the target antigen. An exemplary low antigen density of CD19 may refer to no more than about 1000 molecules for the TCR or CAR in its microenvironment (e.g., 1000 molecules on every target cell for CAR activation or every antigen-presenting cell for TCR activation). Another exemplary low density for antigens may refer to from no more than about 100 to no more than about 10 million antigen molecules. In some embodiments, a low antigen density may refer to no more than about 10, 50, 100, 200, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 150000, 200000, 250000, 300000, 350000, 400000, 450000, 500000, 550000, 600000, 650000, 700000, 750000, 800000, 850000, 900000, 950000, 1 million, 2 million, 3 million, 4 million, 5 million, 6 million, 7 million, 8 million, 9 million, or 10 million molecules of a target antigen. In some embodiments, a low antigen density may refer to no more than 70%, 60%, 50%, 40%, 30%, 20%, 10%, 5%, 1%, 0.5%, 0.1%, 0.05%, 0.01%, or less molecules of a target antigen compared to a normal or high antigen density (e.g., the density of a target antigen expressed on a wild-type target cell). The density or the amount of a target antigen may be measured by various methods, such as semi-quantitative FACS, immunohistochemistry, or other immunological methods. SLP-76 polypeptides, e.g., membrane-bound SLP-76 polypeptides described herein, are also capable of enhancing cell activation when the target antigens are in a normal or high density. In a non-limiting example, overexpression of a CAR or TCR molecule in a cell may result in cell exhaustion. SLP-76 overexpression may enhance CAR or TCR capacity without exacerbating T cell exhaustion. In some embodiments, a CAR or TCR or other receptor molecule expressed on a cell may have a low affinity to its target antigen, thus resulting in weak activation even when there are a plenty of target antigens in its microenvironment. In some embodiments, SLP-76 overexpression may enhance capacity of these CARs or TCRs or other receptors having a low affinity to target antigens, thus enhancing cell activation. In some non-limiting embodiments, a CAR or TCR or other receptor molecule expressed on a cell may be expressed at a low density, thus resulting in weak activation of the cell even when there are a plenty of target antigens in its microenvironment. In some non-limiting embodiments, SLP-76 overexpression may enhance capacity of these CARs or TCRs or other receptors having a low affinity to target antigens, thus enhancing cell activation. In some non-limiting embodiments, a cell (e.g., a cancer or tumor or another target cell) related to a disease or disorder may express a low level of an antigen (i.e., having a low antigen density), which may be insufficient to activate a CAR or TCR molecule recognizing the antigen, thus rendering a common CAR T or TCR therapy using such CAR or TCR molecule insufficient or ineffective to treat the disease or disorder. In some non-limiting embodiments, SLP-76 (e.g., membrane-bound SLP-76 constructs) overexpression may enhance the activation and function of these CARs or TCRs or other receptors, thus enabling the CAR T or TCR therapy to treat the disease or disorder.

### Hinge Domains

As described above, the CAR polypeptides described herein may have an optional hinge domain. Within a chimeric antigen receptor, the term “hinge domain” generally refers to a flexible polypeptide connector region disposed between the targeting moiety (ECD) and the TMD. These sequences are generally derived from IgG subclasses (such as IgG1 and IgG4), IgD and CD8 domains, of which IgG1 has been most extensively used.

In some embodiments, the hinge domain provides structural flexibility to flanking polypeptide regions. The hinge domain may consist of natural or synthetic polypeptides. In recent years, several studies of the hinge domain mainly focused on the following aspects: (1) reducing binding affinity to the Fcγ receptor, thereby eliminating certain types of off-target activation; (2) enhancing the single-chain variable fragment (scFv) flexibility, thereby relieving the spatial constraints between particular epitopes targeted on tumor antigens and the CAR's antigen-targeting moiety; (3) reducing the distance between an scFv and the target epitope(s); and (4) facilitating the detection of CAR expression using anti-Fc reagents. Nevertheless, the influences of the hinge domain on CAR T cell physiology are not well understood.

It can be appreciated by those skilled in the art that hinge domains may improve the function of the CAR polypeptides described herein by promoting optimal positioning of the antigen-binding moiety in relationship to the portion of the antigen recognized by the same. It can be appreciated that, in some embodiments, the hinge domain may not be required for optimal CAR activity. In some embodiments, a beneficial hinge domain having a short sequence of amino acids promotes CAR activity by facilitating antigen binding by, e.g., relieving any steric constraints that may otherwise alter antibody binding kinetics. The sequence encoding the hinge domain may be positioned between the antigen recognition moiety and the TMD. In some embodiments, the hinge domain is operably linked downstream of the antigen-binding moiety and upstream of the TMD. In case a CAR polypeptide described herein has an optional hinge domain, the format to describe the hinge domain and the TMD domain may be “H/TM” or “H-TM”.

The hinge sequence can generally be any moiety or sequence derived or obtained from any suitable molecule. For example, in some embodiments, the hinge sequence can be derived from the human CD8 molecule or a CD28 molecule and any other receptors that provide a similar function in providing flexibility to flanking regions. The hinge domain can have a length of from about 4 amino acid (aa) to about 50 aa, e.g., from about 4 aa to about 10 aa, from about 10 aa to about 15 aa, from about aa to about 20 aa, from about 20 aa to about 25 aa, from about 25 aa to about 30 aa, from about 30 aa to about 40 aa, or from about 40 aa to about 50 aa. Suitable hinge domains can be readily selected and can be of any of a number of suitable lengths, such as from 1 amino acid (e.g., Gly) to 20 aa, from 2 aa to 15 aa, from 3 aa to 12 aa, including 4 aa to 10 aa, 5 aa to 9 aa, 6 aa to 8 aa, or 7 aa to 8 aa, and can be 1, 2, 3, 4, 5, 6, or 7 aa. Non-limiting examples of suitable hinge domains include a CD8 hinge domain, a CD28 hinge domain, a CD4 hinge domain, a PD-1 hinge domain, a CD2 hinge domain, a CTLA4 hinge domain, or an IgG4 hinge domain. In some embodiments, the hinge domain can include regions derived from a human CD8a (a.k.a. CD8a) molecule or a CD28 molecule and any other receptors that provide a similar function in providing flexibility to flanking regions. Additional exemplary hinge domains derive from or include hinge domains of LFA-1 (CD11a/CD18), CD5, CD27 (TNFRSF7), CD70, 4-1BB, OX40 (CD134), ICOS (CD278), IgG1 Fc region, IgG2 Fc region, IgG3 Fc region, IgG4 Fc region, IgE Fc region, IgM Fc region, IgA Fc region, or a combination thereof. In some embodiments, the CAR disclosed herein includes a hinge domain derived from a CD8 hinge domain. In some embodiments, the hinge domain can include one or more copies of the CD8 hinge domain. In some embodiments, the CAR disclosed herein includes a hinge domain derived from a CD28 hinge domain. In some embodiments, the hinge domain can include one or more copies of the CD28 hinge domain. In some embodiments, the CAR disclosed herein includes a hinge domain derived from a CD4 hinge domain. In some embodiments, the hinge domain includes one or more copies of the CD4 hinge domain. In some embodiments, the CAR disclosed herein includes a hinge domain derived from an IgG4 hinge domain. In some embodiments, the hinge domain can include one or more copies of the IgG4 hinge domain.

### Costimulatory Domains

As described above, the CAR polypeptides described herein may have an optional costimulatory domain. Generally, the costimulatory domain suitable for the CAR polypeptides disclosed herein can be any one of the costimulatory domains known in the art. Examples of suitable costimulatory domains that can enhance cytokine production and include, but are not limited to, costimulatory polypeptide sequences derived from 4-1BB (CD137), CD27, CD28, OX40 (CD134), and costimulatory inducible T-cell co-stimulator (ICOS) polypeptide sequences. Additional exemplary costimulatory polypeptide sequences may be or be derived from a costimulatory domain of: CD28, ICOS (CD278), CD27, 4-1BB (CD137), OX40 (CD134), CD2, CD4, CD5, CD7, CD8, CD8a, CD80, CD11a, CD11b, CD11c, CD11d, CD18, CD19, CD19a, CD29, CD30, CD30L, CD40, CD40L (CD154), CD48, CD49a, CD49D, CD49f, CD58, CD53, ICAM-1 (CD54), CD69, CD70, CD80 (B7-1), CD82, CD83, CD84, CD86 (B7-2), CD90, CD96, CD100, CD103, CD122, CD132, CD150 (SLAMFI), CD160 (BY55), CD162 (DNAM1), CD223 (LAG3), CD226, CD229, CD244, CD270 (HVEM), CD273 (PD-L2), CD274 (PD-L1), CD278, LAT, lymphocyte function-associated antigen-1 (LFA-1), LIGHT, NKG2C, NKG2D, NKp30, NKp44, NKp46, NKp80 (KLRF1), B7-H2, B7-H3, CD83 ligand, PD-1, SLP-76, Toll-like receptors (TLRs, such as TLR2), DAP10, DAP12, LAG-3, 2B4, CARD1, CTLA-4 (CD152), TRIM, ZAP70, FcERIγ, 4-1BBL, BAFF, GADS, GITR, GITR-L, BAFF-R, HVEM, CD27L, OX40L, TACi, BLAME, CRACC, CD2F-10, NTB-A, integrin α4, integrin α4β1, integrin a407, IA4, ICAM-1, IL2Rβ, IL2Rγ, IL7Rα, ITGA4, ITGA6, ITGAD, ITGAE, ITGAL, ITGAM, ITGAX, ITGB1, ITGB2, ITGB7, KIRDS2, LTBR, PAG/Cbp, PSGL1, SLAMF6 (NTB-A, Ly108), SLAMF7, SLP-76, TNFR2, TRANCE/RANKL, VLA1, VLA-6, BTLA, ikaros, LAG-3, LMIR, CEACAMI, CRTAM, TCL1A, DAP12, TIM-1, Dectin-1, PDCD6, PD-1, TIM-4, TSLP, EphB6, TSLP-R, HLA-DR, or any combination thereof.

Accordingly, in some embodiments, the costimulatory domain of the CARs disclosed herein is selected from the group consisting of a costimulatory 4-1BB (CD137) polypeptide sequence, a costimulatory CD27 polypeptide sequence, a costimulatory CD28 polypeptide sequence, a costimulatory OX40 (CD134) polypeptide sequence, and a costimulatory inducible T-cell co-stimulator (ICOS) polypeptide sequence. In some embodiments, the CARs disclosed herein include a costimulatory domain derived from a costimulatory 4-1BB (CD137) polypeptide sequence. In some embodiments, the CARs disclosed herein include a costimulatory 4-1BB (CD137) polypeptide sequence. In some embodiments, the CARs disclosed herein include a costimulatory domain derived from a costimulatory CD28 polypeptide sequence. In some embodiments, the CARs disclosed herein include a costimulatory CD28 polypeptide sequence. In some embodiments, the CARs disclosed herein include a costimulatory domain having an amino acid sequence exhibiting at least 50% sequence identity to the sequence of any costimulatory domain described herein. In some embodiments, the costimulatory domain has an amino acid sequence exhibiting at least 50%, at least 60%, at least 70%, at least 80%, at least 85%, at least 90%, at least 95%, at least 96%, at least 97%, at least 98%, at least 99%, or 100% sequence identity to the sequence of any costimulatory domain described herein.

### Transmembrane Domains (TMD)

Generally, the transmembrane domain (also referred to as transmembrane region) suitable for the CAR polypeptides disclosed herein can be any one of the TMDs known in the art. Without being bound to theory, it is believed that the TMD traverses the cell membrane, anchors the CAR to the cell surface, and connects the ECD to the intracellular signaling domain, thus impacting expression of the CAR on the cell surface. Examples of suitable TMDs include, but are not limited to, a CD28 TMD, a CD8 TMD, a CD4 TMD, a CD3 TMD, a CTLA-4 TMD, an OX40 TMD, a 4-1BB TMD, a CD2 TMD, and a PD-1 TMD. Additional exemplary TMDs include TMDs from CD3D, CD3E, CD3G, CD3zeta, CD8a, CD8b, CD16, CD25, CD27, CD40, CD79A, CD79B, CD80, CD84, CD86, CD95, CD150 (SLAMFI), CD166, CD200R, CD223 (LAG3), CD270 (HVEM), CD272 (BTLA), CD273 (PD-L2), CD274 (PD-L1), CD278 (ICOS), CD300, CD357 (GITR), A2aR, ICAM-1, 2B4, BTLA, DAP10, FcRa, FcRP, Fyn, GAL9, IL7, IL12, IL15, KIR, KIR2DL4, KIR2DS1, LAG-3, Lck, LAT, LPA5, LRP, NKp30, NKp44, NKp46, NKG2C, NKG2D, NOTCH1, NOTCH2, NOTCH3, NOTCH4, PTCH2, ROR2, Ryk, SLP-76, SIRPa, pTa, T-cell receptor polypeptides (e.g., TCRa and TCRP), TIM3, TRIM, and ZAP70. Accordingly, in some embodiments, the TMD is derived from a CD28 TMD, a CD8 TMD, a CD4 TMD, a CD3 TMD, a CTLA4 TMD, an OX40 TMD, a 4-1BB TMD, a CD2 TMD, and a PD-1 TMD. In some embodiments, the TMD includes a CD28 TMD, a CD4 TMD, a CD8 TMD, a CD3 TMD, a CTLA4 TMD, an OX40 TMD, a 4-1BB TMD, a CD2 TMD, and a PD-1 TMD. In some embodiments, the CAR disclosed herein include a TMD derived from a CD8. In some embodiments, the CAR polypeptides disclosed herein include a CD8 TMD. In some embodiments, the CAR disclosed herein include a TMD derived from a CD28. In some embodiments, the CAR disclosed herein include a CD28 TMD. In some embodiments, the CAR disclosed herein include a TMD derived from a CD4. In some embodiments, the CAR disclosed herein include a CD4 TMD.

Exemplary CAR molecules as described herein contain a hinge domain and a TMD domain adjacent to each other. Sequences are disclosed herein for exemplary hinge/transmembrane (H/TM or hinge/TM) domains for various CAR molecules. In some embodiments, a CAR molecule disclosed herein includes an H/TM domain having an amino acid sequence exhibiting at least 50% sequence identity to any TMD or Hinge-TMD described herein. In some embodiments, a CAR molecule disclosed herein includes an H/TM domain having an amino acid sequence exhibiting at least 50%, at least 60%, at least 70%, at least 80%, at least 85%, at least 90%, at least 95%, at least 96%, at least 97%, at least 98%, at least 99%, or 100% sequence identity to any TMD or Hinge-TMD described herein.

### Extracellular Spacer

The CARs disclosed herein may further include an optional extracellular spacer domain including one or more intervening amino acid residues that are positioned between the ECD and an optional hinge domain. In some embodiments, the extracellular spacer domain is operably linked downstream to the ECD and upstream to an optional hinge domain. In principle, there are no particular limitations to the length and/or amino acid composition of the extracellular spacer. In some embodiments, any arbitrary single-chain peptide including about one to 100 amino acid residues (e.g., 2, 3, 4, 5, 6, 7, 8, 9, 10, 11, 12, 13, 14, 15, 16, 17, 18, 19, 20, etc. amino acid residues) can be used as an extracellular spacer. In some embodiments, the extracellular spacer includes about 5 to 50, about 10 to 60, about 20 to 70, about 30 to 80, about 40 to 90, about 50 to 100, about 60 to 80, about 70 to 100, about 30 to 60, about 20 to 80, about 30 to 90 amino acid residues. In some embodiments, the extracellular spacer includes about 1 to 10, about 5 to 15, about 10 to 20, about 15 to 25, about 20 to 40, about 30 to 50, about 40 to 60, about 50 to 70 amino acid residues. In some embodiments, the extracellular spacer includes about 40 to 70, about 50 to 80, about 60 to 80, about 70 to 90, or about 80 to 100 amino acid residues. In some embodiments, the extracellular spacer includes about 1 to 10, about 5 to 15, about 10 to 20, about 15 to 25 amino acid residues. In some embodiments, the length and amino acid composition of the extracellular spacer can be optimized to vary the orientation and/or proximity of the ECD and an optional hinge domain to one another to achieve a desired activity of the CARs. In some embodiments, the orientation and/or proximity of the ECD and an optional hinge domain to one another can be varied and/or optimized as a “tuning” tool or effect that would enhance or reduce the efficacy of the CARs. In some embodiments, the orientation and/or proximity of the ECD and an optional hinge domain to one another can be varied and/or optimized to create fully functional or partially functional versions of the CARs. In some embodiments, the extracellular spacer domain includes an amino acid sequence corresponding to an IgG4 hinge domain and an IgG4 CH2-CH3 domain. Additional exemplary extracellular spacer domains may derive from or include an immunoglobulin hinge region (e.g., IgG1, IgG2, IgG3, IgG4, IgA, IgD), all or a portion of an immunoglobulin Fc domain (e.g., a CH1 domain, a CH2 domain, a CH3 domain, or combinations thereof), a stalk region of a type II C-lectin (the extracellular domain located between the C-type lectin domain and the transmembrane domain). Type II C-lectins include, e.g., CD23, CD69, CD72, CD94, NKG2A, and NKG2D. In yet further embodiments, an extracellular spacer domain may be derived from or include a toll-like receptor (TLR) juxtamembrane domain. A TLR juxtamembrane domain contains acidic amino acids lying between the leucine rich repeats (LRRs) and the transmembrane domain of a TLR. In certain embodiments, a TLR juxtamembrane domain is a TLR1, TLR2, TLR3, TLR4, TLR5, TLR6, TLR7, TLR8, or TLR9 juxtamembrane domain.

One skilled in the art can appreciate that the complete amino acid sequence of a CAR polypeptide of the disclosure can be used to construct a back-translated gene. For example, a DNA oligomer containing a nucleotide sequence coding for a given CAR can be synthesized. For example, several small oligonucleotides coding for portions of the desired CAR or antibody can be synthesized and then ligated. The individual oligonucleotides typically contain 5′ or 3′ overhangs for complementary assembly.

In addition to generating desired CARs via expression of nucleic acid molecules that have been altered by recombinant molecular biological techniques, a subject CAR in accordance with the present disclosure can be chemically synthesized. Chemically synthesized polypeptides are routinely generated by those of skill in the art.

Once assembled (by synthesis, recombinant methodologies, site-directed mutagenesis or other suitable techniques), the DNA sequences encoding a CAR as disclosed herein can be inserted into an expression vector and operably linked to an expression control sequence appropriate for expression of the CAR in the desired transformed host. Proper assembly can be confirmed by nucleotide sequencing, restriction mapping, and expression of a biologically active polypeptide in a suitable host. As is known in the art, in order to obtain high expression levels of a transfected gene in a host, take should be taken to ensure that the gene is operably linked to transcriptional and translational expression control sequences that are functional in the chosen expression host.

### Intracellular Signaling Domain

The CARs disclosed herein may include an intracellular signaling domain to transduce signaling from the ECD binding to target antigens and to induce cell activation.

Within a chimeric antigen receptor (CAR), the intracellular signaling domain (a.k.a., cytosolic signaling domain) generally refers to a cytoplasmic domain that transmits an activation signal to a cell expressing the CAR molecule, following binding of the extracellular domain to the corresponding ligand or antigen. In some cases, the intracellular signaling domain includes a functional signaling domain derived from a stimulatory molecule. Traditional intracellular signaling domains almost always include CD3zeta, the prototypical “master switch” that elicits T cell activity (Irving and Weiss Cell 1991; 64:891-901; Letourneur and Klausner Science 1992; 255:79-82), as well as various optional costimulatory domains to enhance potency and persistence.

In some embodiments, the CARs disclosed herein have an intracellular signaling domain from or derived from a proximal signaling molecule. In some embodiments, the CARs disclosed herein have an intracellular signaling domain with an immune receptor tyrosine based activation motif (ITAM). In some embodiments, the CARs disclosed herein have an intracellular signaling domain without an immune receptor tyrosine based activation motif (ITAM). Exemplary proximal signaling molecules may include, e.g., CD3zeta (CD3ζ), CD3-epsilon, DAP12, ZAP70, PLCG1, PKC, ITK, NCK, VAV1, GRB2, GADS, SOS1, ADAP, SYK, LYN, PI3K, BLNK, or a biologically active fragment, mutant, or variant thereof.

### T-Cell Receptors (TCRs)

The T-cell receptor (TCR) is a protein complex found on the surface of T cells, or T lymphocytes, that is responsible for recognizing fragments of antigen as peptides bound to major histocompatibility complex (MHC) molecules. The binding between TCR and antigen peptides is of relatively low affinity and is degenerate. The TCR is composed of two different protein chains (i.e., as a heterodimer). In humans, in 95% of T cells the TCR consists of an alpha (α) chain and a beta (β) chain (encoded by TRA and TRB, respectively), whereas in 5% of T cells the TCR consists of gamma and delta (γ/δ) chains (encoded by TRG and TRD, respectively). Within these chains are complementary determining regions (CDRs) which determine the antigen to which the TCR will bind. Antigen presenting cells (APCs) digest pathogens and display their fragments on major histocompatibility complex (MHC) molecules. This MHC/antigen complex binds to the TCR while other co-stimulatory molecules (e.g. CD28) are activated leading to T cell activation, proliferation, differentiation, apoptosis, or cytokine release. MHC/antigen complexes are, however, not the only molecules capable of interaction with TCRs. Non-peptide antigens such as lipids can interact with TCRs via some of the five isoforms of CD1, and several studies describe TCRs binding to metabolic intermediates bound to the MHC like molecule MR1.

Stimulation of T cell function may be initiated upon interaction of the TCR with short peptides presented by MHC class I or II molecules (MHC 1 for CD8 T cells and MHC II for CD4 T cells). However, the TCR heterodimer by itself is incapable of activating downstream pathways to initiate T cell activation. Initiation of TCR signaling requires co-receptors such as CD4 for helper T cells and CD8 for cytotoxic T cells. These co-receptors act as cellular adhesion molecules that bind their respective MHC molecules and stabilize the interaction of T cells and antigen presenting cells.

The TCR is also located in close proximity to CD3 family of proteins (CD3δ, CD3ε, and CD3γ) as well as a TCR zeta (ζ) chain. Once the TCR is properly engaged with the peptide-MHC complex, conformational changes in the associated CD3 chains are induced, which leads to their phosphorylation and signaling through proximal signaling molecules, similar to the signaling with CAR molecules.

As described above, the SLP-76 polypeptides of the present disclosure are capable of enhancing activation of cells (e.g., immune cells, such as T cells) expressing TCRs (either native or recombinant) in response to target antigens presented by MHC molecules on antigen-presenting cells. In principle, there are no particular limitations with regard to suitable TCR molecules for the enhancement function of the SLP-76 polypeptides described herein. In non-limiting embodiments, the SLP-76 polypeptides described herein are capable of enhancing activation of TCR-expressing cells in response to target antigens presented by, e.g., antigen-presenting cells (APCs). In some embodiments, SLP-76 polypeptides, e.g., the membrane-bound SLP-76 polypeptides described herein, are capable of activating or enhancing activation of TCR-expressing cells in response to target antigens at low densities. In some embodiments, the activation of TCR-expressing cells may enhance cell proliferation, differentiation, cytokine production and/or cytotoxicity. For example, in adoptive cell therapy (ACT), a patient's white blood cells are collected in a process called leukapheresis. Isolation of the specific T cells with the most cancer-fighting potency may be followed by in vitro culture of the cells. This hugely expanded T cell population may be then reinfused into the patient to work against the cancer. In some embodiments, SLP-76 polypeptides, e.g., membrane-bound SLP-76 polypeptides described herein, may be capable of activating or enhancing activation and/or proliferation of TCR-expressing cells in response to presented target antigens (e.g., tumor antigens), even when the target antigens are expressed at a low density on antigen-presenting cells. Therefore, SLP-76 polypeptides described herein may be used to selectively enhance proliferation and/or isolation of TCR-expressing cells with specificity and/or potency to target antigens (e.g., tumor antigens).

### Mutations to Chimeric Antigen Receptors (CARs) or T-Cell Receptors (TCRs)

Multiple mutations can be introduced to the CAR or TCR polypeptides of the present disclosure. Mutations include, at least, substitution, deletion, insertion, or other methods known in the art. The purpose of mutations may include, for example, to enhance the potency of the CAR or TCR polypeptide, to enhance the stability (e.g., half-life) of the CAR or TCR polypeptide, to enhance the expression of the CAR or TCR polypeptide, to enhance the solubility and/or to reduce aggregation of the CAR or TCR polypeptide, to manipulate modifications of the CAR or TCR polypeptide during expression, to manipulate binding of the CAR or TCR polypeptide to its binding partner(s), to enhance the purification of the CAR or TCR polypeptide, to reduce ubiquitination and/or degradation of the CAR or TCR polypeptide, etc.

### Nucleic Acid Molecules

In one aspect, provided herein are various nucleic acid molecules including nucleotide sequences encoding a SLP-76 polypeptide, a CAR polypeptide, or a TCR polypeptide of the disclosure, including expression cassettes and expression vectors containing these nucleic acid molecules operably linked to heterologous nucleic acid sequences such as, for example, regulator sequences which allow in vivo expression of the SLP-76 polypeptide, the CAR polypeptide, or the TCR polypeptide in a host cell or ex-vivo cell-free expression system.

Nucleic acid molecules of the present disclosure can be nucleic acid molecules of any length, including nucleic acid molecules that are generally between about 0.5 Kb and about 50 Kb, for example between about 0.5 Kb and about 20 Kb, between about 1 Kb and about 15 Kb, between about 2 Kb and about 10 Kb, or between about 5 Kb and about 25 Kb, for example between about 10 Kb to 15 Kb, between about 15 Kb and about 20 Kb, between about 5 Kb and about 20 Kb, about 5 Kb and about 10 Kb, or about 10 Kb and about 25 Kb. In some embodiments, the nucleic acid molecules of the disclosure are between about 1.5 Kb and about 50 Kb, between about 5 Kb and about 40 Kb, between about 5 Kb and about 30 Kb, between about 5 Kb and about 20 Kb, or between about 10 Kb and about 50 Kb, for example between about 15 Kb to 30 Kb, between about 20 Kb and about 50 Kb, between about 20 Kb and about 40 Kb, about 5 Kb and about 25 Kb, or about 30 Kb and about 50 Kb.

In some embodiments, the recombinant nucleic acid includes a nucleic acid sequence encoding a SLP-76 polypeptide, a CAR polypeptide, or a TCR polypeptide.

In some embodiments, a composition has at least two recombinant nucleic acids, each including a nucleic acid sequence encoding a SLP-76 polypeptide described herein and a CAR or a TCR polypeptide described herein to form a combination. In some embodiments, these at least two recombinant nucleic acids are conjugated together. In some embodiments, these at least two recombinant nucleic acids are in a single chain of a recombinant nucleic acid.

In some embodiments, the recombinant nucleic acid encodes an amino acid sequence having at least 50% sequence identity to an amino acid sequence selected from the group consisting of SEQ ID NOs: 1, 7, 14, 16, 18, 19, 40, 59 or 60. In some embodiments, the recombinant nucleic acid encodes an amino acid sequence having at least 50%, at least 60%, at least 70%, at least 80%, at least 85%, at least 90%, at least 95%, at least 96%, at least 97%, at least 98%, at least 99%, or 100% sequence identity to an amino acid sequence selected from the group consisting of SEQ ID NOs: 1, 7, 14, 16, 18, 19, 40, 59 or 60. In some embodiments, the recombinant nucleic acid includes a nucleic acid sequence having at least 50% sequence identity to a nucleic acid sequence selected from the group consisting of SEQ ID NOs: 20, 26, 33, 35, 37, 38, 42, 61 and 62. In some embodiments, the recombinant nucleic acid includes a nucleic acid sequence having at least 50%, at least 60%, at least 70%, at least 80%, at least 85%, at least 90%, at least 95%, at least 96%, at least 97%, at least 98%, at least 99%, or 100% sequence identity to a nucleic acid sequence selected from the group consisting of SEQ ID NOs: 20, 26, 33, 35, 37, 38, 42, 61 and 62.

In some embodiments, the recombinant nucleic acid molecule is operably linked to a heterologous nucleic acid sequence.

In some embodiments, the recombinant nucleic acid molecule is further defined as an expression cassette or a vector. It can be understood that an expression cassette generally includes a construct of genetic material that contains coding sequences and enough regulatory information to direct proper transcription and/or translation of the coding sequences in a recipient cell, in vivo and/or ex vivo. Generally, the expression cassette may be inserted into a vector for targeting to a desired host cell and/or into an individual. As such, in some embodiments, an expression cassette of the disclosure includes a coding sequence for the SLP-76, CAR, or TCR polypeptide as disclosed herein, which is operably linked to expression control elements, such as a promoter, and optionally, any other sequences or a combination of other nucleic acid sequences that affect the transcription or translation of the coding sequence.

In some embodiments, the nucleotide sequence is incorporated into an expression vector. It can be understood by one skilled in the art that the term “vector” generally refers to a recombinant polynucleotide construct designed for transfer between host cells, and that may be used for the purpose of transformation, e.g., the introduction of heterologous DNA into a host cell. As such, in some embodiments, the vector can be a replicon, such as a plasmid, phage, or cosmid, into which another DNA segment may be inserted so as to bring about the replication of the inserted segment. In some embodiments, the expression vector can be an integrating vector.

In some embodiments, the expression vector can be a viral vector. As can be appreciated by one of skill in the art, the term “viral vector” is widely used to refer either to a nucleic acid molecule (e.g., a transfer plasmid) that includes virus-derived nucleic acid elements that generally facilitate transfer of the nucleic acid molecule or integration into the genome of a cell or to a viral particle that mediates nucleic acid transfer. Viral particles generally include various viral components and sometimes also host cell components in addition to nucleic acid(s). The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. In some embodiments, the vector is a vector derived from a lentivirus, an adeno virus, an adeno-associated virus, a baculovirus, or a retrovirus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus, which is a genus of retrovirus.

In some embodiments, provided herein are nucleic acid molecules encoding a polypeptide with an amino acid sequence having at least about 50%, 60%, 65%, 70%, 75%, 80%, 85%, 90%, 95%, 96%, 97, 98%, 99%, or 100% sequence identity to a CAR, TCR, or another receptor polypeptide disclosed herein.

The nucleic acid sequences encoding the CAR polypeptides can be optimized for expression in the host cell of interest. For example, the G-C content of the sequence can be adjusted to average levels for a given cellular host, as calculated by reference to known genes expressed in the host cell. Methods for codon usage optimization are known in the art. Codon usages within the coding sequence of the chimeric receptor disclosed herein can be optimized to enhance expression in the host cell, such that about 1%, about 5%, about 10%, about 25%, about 50%, about 75%, or up to 100% of the codons within the coding sequence have been optimized for expression in a particular host cell.

The nucleic acid molecules provided can contain naturally occurring sequences, or sequences that differ from those that occur naturally, but, due to the degeneracy of the genetic code, encode the same polypeptide, e.g., antibody for the ECD. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (e.g., either a sense or an antisense strand).

The nucleic acid molecules are not limited to sequences that encode polypeptides (e.g., antibodies for the ECD); some or all of the non-coding sequences that lie upstream or downstream from a coding sequence (e.g., the coding sequence of a chimeric receptor) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

### Recombinant Cells and Cell Cultures

The nucleic acid molecules of the present disclosure can be introduced into a cell (i.e., a host cell), such as an immune cell, including, e.g., a T cell, to produce a recombinant cell containing the nucleic acid molecule. In principle, there are no particular limitations with regard to suitable cells for expressing the nucleic acid molecules of the present disclosure. Accordingly, some embodiments of the disclosure relate to methods for making a recombinant cell, including (a) providing a host cell capable of protein expression; and transducing the provided host cell with a recombinant nucleic acid of the disclosure to produce a recombinant cell. Introduction of the nucleic acid molecules of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

In one aspect, the instant disclosure provides a composition having an isolated recombinant cell modified to overexpress and/or contain elevated levels of a SLP-76 polypeptide. In some embodiments, the recombinant cell is capable of being activated by a target antigen expressed at a low density. In some embodiments, the recombinant cell is a cell expressing a SLP-76 polypeptide described herein. In some embodiments, the recombinant cell is a cell expressing a SLP-76 polypeptide described herein and further expressing a CAR or TCR as described in previous sections. For example, a SLP-76 construct may be introduced into a cell with or without expressing a CAR or TCR to produce a recombinant cell overexpressing SLP-76 (or further overexpressing the CAR or TCR), thus enhancing cell activation through promoting proximal signaling and/or through enhancing the CAR or TCR capacity to activate the cell. In some embodiments, the recombinant cell is a cell expressing a CAR or TCR. In some embodiments, a SLP-76 construct may be introduced into the cell expressing a CAR or TCR for overexpressing SLP-76. In some embodiments, the recombinant cell is a cell not expressing a CAR or TCR. In some embodiments, a SLP-76 construct may be introduced into the cell not expressing a CAR or TCR to enhance cell activation. In non-limiting embodiments, a SLP-76 construct may enhance activation of a cell not expressing a CAR or TCR by promoting proximal signaling in the cell. In some embodiments, the cell not expressing a CAR or TCR is a NK cell. In some embodiments, a SLP-76 construct may be introduced in combination with a CAR or TCR construct (either sequentially or together) into a cell not expressing a CAR or TCR to produce a recombinant cell overexpressing SLP-76 and the CAR or TCR, thus enhancing the CAR or TCR capacity to activate the cell. In non-limiting embodiments, SLP-76 may be introduced into a native host cell or a recombinant cell for overexpression. In some embodiments, SLP-76 may be introduced into a native host cell or a recombinant cell expressing a native or endogenous TCR. In some embodiments, the overexpressed SLP-76 may enhance cell activation through the native or endogenous TCR. In some embodiments, SLP-76 may be introduced into a recombinant cell expressing a recombinant TCR or CAR. In some embodiments, the overexpressed SLP-76 may enhance cell activation through the recombinant TCR or CAR.

Accordingly, in some embodiments, the nucleic acid molecules can be introduced into a host cell by viral or non-viral delivery vehicles known in the art to produce an engineered cell. For example, the nucleic acid molecule can be stably integrated in the host genome, or can be episomally replicating, or present in the recombinant host cell as a mini-circle expression vector for a stable or transient expression. Accordingly, in some embodiments disclosed herein, the nucleic acid molecule is maintained and replicated in the recombinant host cell as an episomal unit. In some embodiments, the nucleic acid molecule is stably integrated into the genome of the recombinant cell. Stable integration can be completed using classical random genomic recombination techniques or with more precise genome editing techniques such as using zinc-finger proteins (ZNF), guide RNA directed CRISPR/Cas9, DNA-guided endonuclease genome editing NgAgo (Natronobacterium gregoryi Argonaute), or TALEN genome editing (transcription activator-like effector nucleases).

The nucleic acid molecules can be encapsulated in a viral capsid or a lipid nanoparticle, or can be delivered by viral or non-viral delivery means and methods known in the art, such as electroporation. For example, introduction of nucleic acids into cells may be achieved by viral transduction. In a non-limiting example, baculoviral virus or adeno-associated virus (AAV) can be engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral-derived vector systems are also useful for nucleic acid delivery and gene therapy via viral transduction. Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile; and (vii) a relatively easy system for vector manipulation and production.

In some embodiments, host cells can be genetically engineered (e.g., transduced or transformed or transfected) with, for example, a vector construct of the present application that can be, for example, a viral vector or a vector for homologous recombination that includes nucleic acid sequences homologous to a portion of the genome of the host cell, or can be an expression vector for the expression of the SLP-76, CAR, and/or TCR polypeptides of interest.

In some embodiments, the recombinant cell is a eukaryotic cell. In some embodiments, the cell is in vivo. In some embodiments, the cell is ex vivo. In some embodiments, the cell is in vitro. In some embodiments, the recombinant cell is an animal cell. In some embodiments, the animal cell is a mammalian cell. In some embodiments, the animal cell is a mouse cell. In some embodiments, the animal cell is a human cell. In some embodiments, the cell is a non-human primate cell. In some embodiments, the recombinant cell is an immune system cell, e.g., a B cell, a monocyte, a NK cell, a tumor-infiltrating lymphocyte (TIL), a natural killer T (NKT) cell, a basophil, an eosinophil, a neutrophil, a dendritic cell, a macrophage, a regulatory T cell (Treg), a helper T cell (T_{H}), a cytotoxic T cell (T_{CTL}), a memory T cell, a gamma delta (γδ) T cell, another T cell, a stem cell (e.g., a hematopoietic stem cell), a stem cell progenitor (e.g., a hematopoietic stem cell progenitor) an induced pluripotent stem cell (iPSC)-derived NK cell, or an induced pluripotent stem cell (iPSC)-derived T cell.

In some embodiments, the immune system cell is a lymphocyte. In some embodiments, the lymphocyte is a T lymphocyte. In some embodiments, the lymphocyte is a T lymphocyte progenitor. In some embodiments, the T lymphocyte is a CD4+ T cell or a CD8+ T cell. In some embodiments, the T lymphocyte is a CD8+T cytotoxic lymphocyte cell. Non-limiting examples of CD8+T cytotoxic lymphocyte cell suitable for the compositions and methods disclosed herein include naïve CD8+ T cells, central memory CD8+ T cells, effector memory CD8+ T cells, effector CD8+ T cells, CD8+ stem memory T cells, and bulk CD8+ T cells. In some embodiments, the T lymphocyte is a CD4+T helper lymphocyte cell. Suitable CD4+T helper lymphocyte cells include, but are not limited to, naïve CD4+ T cells, central memory CD4+ T cells, effector memory CD4+ T cells, effector CD4+ T cells, CD4+ stem memory T cells, and bulk CD4+ T cells.

In some embodiments, the cell described herein is a non-immune system cell. For example, the SLP-76, CAR, or TCR molecules described herein provide a method to modulate the activity of (e.g., to activate) a cell expressing such molecules, upon recognizing the corresponding extracellular ligand(s)/antigen(s). In this sense, there are no particular limitations with regard to suitable host cell.

The cells described herein may be any type of natural or artificial cells and/or of any origins. In principle, any type of cells that people want to study the activation may be used for expressing SLP-76, CAR, and/or TCR polypeptides described herein. For preventing or treating a disease described herein, cells having cytotoxicity or other inhibitory functions (e.g., functions to produce and secrete cytokines) to a target cell correlated to the disease may be used. For example, immune cells, such as T cells, may be used to express the polypeptides, because the cells may have a direct cytotoxic capacity to a tumor cell expressing a tumor antigen, and/or the cells may produce at least one cytokine to inhibit or kill a target cell (e.g., a tumor cell).

As outlined above, some embodiments of the disclosure relate to various methods for making a recombinant cell, including (a) providing a host cell capable of protein expression; and transducing the provided host cell with a recombinant nucleic acid of the disclosure to produce a recombinant cell. Non-limiting exemplary embodiments of the disclosed methods for making a recombinant cell can further include one or more of the following features. In some embodiments, the host cell is obtained by leukapheresis performed on a sample obtained from a subject, and the cell is transduced ex vivo. In some embodiments, the recombinant nucleic acid is encapsulated in a viral capsid or a lipid nanoparticle. In some embodiments, the methods further include isolating and/or purifying the produced cells. Accordingly, the recombinant cells produced by the methods disclosed herein are also within the scope of the disclosure.

Techniques for transforming a wide variety of the above-mentioned host cells and species are known in the art and described in the technical and scientific literature. For example, DNA vectors can be introduced into eukaryotic cells via conventional transformation or transfection techniques. Suitable methods for transforming or transfecting cells can be found in Sambrook et al. (2012, supra) and other standard molecular biology laboratory manuals, such as, calcium phosphate transfection, DEAE-dextran mediated transfection, transfection, microinjection, cationic lipid-mediated transfection, electroporation, transduction, scrape loading, ballistic introduction, nucleoporation, hydrodynamic shock, and infection. In some embodiments, the nucleic acid molecule is introduced into a host cell by a transduction procedure, electroporation procedure, or a biolistic procedure. Accordingly, cell cultures including at least one recombinant cell as disclosed herein are also within the scope of this application. Methods and systems suitable for generating and maintaining cell cultures are known in the art.

In some embodiments, the recombinant cell includes a nucleic acid molecule including a nucleic acid sequence encoding a SLP-76 polypeptide having at least 50%, 60%, 70%, 80%, 90%, 95%, 96%, 97, 98%, 99%, or 100% sequence identity to a SLP-76 polypeptide disclosed herein. In some embodiments, the recombinant cell includes a nucleic acid molecule encoding a polypeptide with an amino acid sequence having at least about 50%, 60%, 70%, 80%, 90%, 95%, 96%, 97, 98%, 99%, or 100% sequence identity to any one of SEQ ID NOs: 1, 7, 14, 16, 18, 19, 40, 59 or 60. In some embodiments, the recombinant cell contains a nucleic acid molecule having a sequence having at least about 50%, 60%, 70%, 80%, 90%, 95%, 96%, 97, 98%, 99%, or 100% sequence identity to any one of SEQ ID NOs: 20, 26, 33, 35, 37, 38, 42, 61 and 62.

In a related aspect, some embodiments of the disclosure relate to cell cultures including at least one recombinant cell as disclosed herein, and a culture medium. Generally, the culture medium can be any one of suitable culture media for the cell cultures described herein. In some embodiments, the recombinant cell expresses a SLP-76, CAR, or TCR described herein. Accordingly, cell cultures including at least one recombinant cell as disclosed herein are also within the scope of this application. Methods and systems suitable for generating and maintaining cell cultures are known in the art.

### Pharmaceutical Compositions

The SLP-76, CAR, and/or TCR polypeptides, nucleic acids, recombinant cells, and/or cell cultures of the disclosure can be incorporated into compositions, including pharmaceutical compositions. Such compositions generally include the polypeptides, nucleic acids, recombinant cells, and/or cell cultures as described herein and a pharmaceutically acceptable carrier. Accordingly, in one aspect, some embodiments of the disclosure relate to pharmaceutical compositions for treating, preventing, ameliorating, reducing or delaying the onset of a health condition, for example a proliferative disease (e.g., cancer). Other exemplary health conditions include, e.g., hematological malignancies, solid tumors, autoimmune diseases, inflammations, allergic diseases, infections, senescence/aging, etc.

Accordingly, one aspect of the present disclosure relates to pharmaceutical compositions that include a pharmaceutically acceptable carrier and one or more of the following: (a) a SLP-76, CAR, and/or TCR polypeptide of the disclosure; (b) a nucleic acid molecule of the disclosure; and/or (c) a recombinant cell of the disclosure. In some embodiments, the composition includes (a) a recombinant nucleic acid of the disclosure and (b) a pharmaceutically acceptable carrier. In some embodiments, the recombinant nucleic acid is encapsulated in a viral capsid or a lipid nanoparticle. In some embodiments, the composition includes (a) a recombinant cell of the disclosure and (b) a pharmaceutically acceptable carrier.

In certain embodiments, the pharmaceutical compositions in accordance with some embodiments disclosed herein include cell cultures that can be washed, treated, combined, supplemented, or otherwise altered prior to administration to an individual in need thereof. Furthermore, administration can be at varied doses, time intervals or in multiple administrations.

The pharmaceutical compositions provided herein can be in any form that allows for the composition to be administered to an individual. In some specific embodiments, the pharmaceutical compositions are suitable for human administration. As used herein, the term “pharmaceutically acceptable” means approved by a regulatory agency of the Federal or a state government or listed in the U.S. Pharmacopeia or other generally recognized pharmacopeia for use in animals, and more particularly in humans. The carrier can be a diluent, adjuvant, excipient, or vehicle with which the pharmaceutical composition is administered. Saline solutions and aqueous dextrose and glycerol solutions can also be employed as liquid carriers, including injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin. In some embodiments, the pharmaceutical composition is sterilely formulated for administration into an individual. In some embodiments, the individual is a human. One of ordinary skilled in the art can appreciate that the formulation should suit the mode of administration.

In some embodiments, the pharmaceutical compositions of the present disclosure are formulated to be suitable for the intended route of administration to an individual. For example, the pharmaceutical composition may be formulated to be suitable for parenteral, intraperitoneal, colorectal, intraperitoneal, and intratumoral administration. In some embodiments, the pharmaceutical composition may be formulated for intravenous, oral, intraperitoneal, intratracheal, subcutaneous, intramuscular, topical, or intratumoral administration.

### Use of the Compositions

The compositions described herein, e.g., SLP-76, CAR, and/or TCR polypeptides, nucleic acids, recombinant cells, cell cultures, and/or pharmaceutical compositions, can be used for various conditions, as a system in response to extracellular signals (e.g., ligands/antigens).

The compositions described herein may also be used as an activation system to manipulate cell functions in response to certain ligands/antigens. In some embodiments, by sensing a specific ligand/antigen or a specific profile of ligands/antigens with the ECD(s) of the CAR or TCR molecules, the host is activated. Such activation may enhance or inhibit the normal biological functions of the host cell, and/or provide an exogenous signaling function to manipulate the cell functions. In some embodiments, SLP-76 compositions described herein may enhance such activation through the CAR or TCR molecules.

The compositions described herein may also be used to manipulate the functions of a cell in response to certain ligands/antigens. In some embodiments, by sensing a specific ligand/antigen with the ECD(s) of the CAR or TCR molecules, the host cell is activated. Such activation may enhance or inhibit the functions of the host cell to change the function of a target cell, which expresses such ligand(s)/antigen(s) or is specifically recognized by certain ligand(s)/antigen(s). In some embodiments, SLP-76 compositions described herein may enhance such activation through the CAR or TCR molecules. For example, a cancer cell expressing certain ligand(s)/antigen(s) may be recognized by the CAR molecule(s) or CAR molecule combination(s) described herein, either through the ECD binding to the ligand(s)/antigen(s) on the surface of the cancer cell or binding to some ligand(s)/antigen(s) which specifically binds to the ligand(s)/antigen(s) expressed on the cancer cell. The activated host cell can then manipulate the function of the cancer cell. For example, the compositions described herein may be used to activate the host cell (e.g., an immune system cell, such as a T cell) to inhibit or kill the cancer cells (e.g., by secreting cytokines or direct killing). In some embodiments, such cancer cells are obtained from a biological sample form a subject. In some embodiments, such cancer cells are in a biological environment (e.g., a cancer microenvironment) of a subject. In some embodiments, a target cell is correlated to a disease or disorder. Exemplary diseases or disorders may include, e.g., proliferative diseases (e.g., cancers), hematological malignancies, solid tumors, autoimmune diseases, inflammations, allergic diseases, infections, senescence/aging, etc.

### Methods of Making

In one aspect, the instant disclosure provides methods of producing a recombination cell as described herein. Compositions described herein, e.g., SLP-75, CAR, and/or TCR polypeptides or nucleic acids, may be introduced into a cell as described herein. For example, a composition comprising a polynucleotide encoding a SLP-76 polypeptide may be introduced into a cell, either alone or in combination with a polynucleotide encoding a T-cell receptor (TCR) polypeptide and/or a chimeric antigen receptor (CAR) polypeptide into a cell.

### Methods of Treatment

In one aspect, the instant disclosure provides methods of enhancing activation of a cell in response to a target antigen, comprising overexpressing a SLP-76 polypeptide in the cell. In some embodiments, the cell further comprises a TCR and/or a CAR molecule.

In another aspect, the instant disclosure provides methods of enhancing activation of a cell in response to a target antigen, comprising overexpressing a SLP-76 polypeptide and a T-cell receptor (TCR) polypeptide and/or a chimeric antigen receptor (CAR) polypeptide in the cell.

In another aspect, the instant disclosure provides methods of treating a disease or disorder in a subject in need of, comprising administering to the subject a pharmaceutically effective amount of a recombinant cell, a polypeptide, a polynucleotide, an expression vector, or other compositions described herein. Administration of any one of the therapeutic compositions described herein, e.g., SLP-76, CARs, and/or TCR polypeptides, nucleic acids, recombinant cells, cell cultures, and/or pharmaceutical compositions, can be used in the diagnosis, prevention, and/or treatment of relevant conditions, such as proliferative diseases (e.g., cancer), hematological malignancies, solid tumors, autoimmune diseases, inflammations, allergic diseases, infections, senescence/aging, etc. In some embodiments, SLP-76, CARs, and/or TCR polypeptides, nucleic acids, recombinant cells, cell cultures, and/or pharmaceutical compositions as described herein can be incorporated into therapies and therapeutic agents for use in methods of preventing and/or treating an individual who has, who is suspected of having, or who may be at high risk for developing one or more health conditions, such as proliferative diseases (e.g., cancers, such as a leukemia, a neuroblastoma, or an osteosarcoma). In some embodiments, the individual is a patient under the care of a physician.

Exemplary proliferative diseases can include, without limitation, angiogenic diseases, a metastatic diseases, tumorigenic diseases, neoplastic diseases and cancers. In some embodiments, the proliferative disease is a cancer. In some embodiments, the cancer is a pediatric cancer. In some embodiments, the cancer is pancreatic cancer, colon cancer, ovarian cancer, prostate cancer, lung cancer, mesothelioma, breast cancer, urothelial cancer, liver cancer, head and neck cancer, sarcoma, cervical cancer, stomach cancer, gastric cancer, melanoma, uveal melanoma, cholangiocarcinoma, multiple myeloma, leukemia, lymphoma, haematological cancer, bladder cancer, neuroblastoma, malignant pleural mesothelioma, sarcoma, and glioblastoma. Exemplary cancer types also include: Acute myeloid leukemia, Angioimmunoblastic T-cell lymphoma, B-cell acute lymphoblastic leukemia, Sweet Syndrome, T-cell Non-Hodgkins lymphoma (including natural killer/T-cell lymphoma, adult T-cell leukaemia/lymphoma, enteropathy type T-cell lymphoma, hepatosplenic T-cell lymphoma and cutaneous T-cell lymphoma), T-cell acute lymphoblastic leukemia, B-cell Non-Hodgkins lymphoma (including Burkitt lymphoma, diffuse large B-cell lymphoma, Follicular lymphoma, Mantle cell lymphoma, Marginal Zone lymphoma, etc.), Hairy Cell Leukemia, Hodgkin lymphoma, Lymphoblastic lymphoma, Lymphoplasmacytic lymphoma, Mucosa-associated lymphoid tissue lymphoma, Multiple myeloma, Myelodysplastic syndrome, Plasma cell myeloma, Primary mediastinal large B-cell lymphoma, chronic myeloproliferative disorders (such as chronic myeloid leukemia, primary myelofibrosis, essential thrombocytemia, polycytemia vera) and chronic lymphocytic leukemia. Exemplary cancer types also include: acute lymphoblastic leukemia (ALL), acute myeloid leukemia (AML), adrenocortical carcinoma, AIDS-related cancers, Kaposi sarcoma (soft tissue sarcoma), AIDS-related lymphoma, primary CNS lymphoma, anal cancer, appendix cancer, astrocytomas, childhood brain cancer, atypical teratoid/rhabdoid tumor, central nervous system cancer, skin cancer (e.g., basal cell carcinoma), bile duct cancer, bladder cancer, bone cancer (includes Ewing sarcoma, osteosarcoma and malignant fibrous histiocytoma), brain tumors, breast cancer, bronchial tumors, Burkitt lymphoma, non-Hodgkin lymphoma, carcinoid tumor, Cardiac (heat) tumors, medulloblastoma and other CNS embryonal tumors, germ cell tumor, Primary CNS Lymphoma, Cervical Cancer, Childhood Cancers, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma, Mycosis Fungoides, Sezary Syndrome, ductal carcinoma in situ (DCIS), Embryonal Tumors, Medulloblastoma, Endometrial Cancer (Uterine Cancer), Ependymoma, Esophageal Cancer, Esthesioneuroblastoma (Head and Neck Cancer), Ewing Sarcoma, Extracranial Germ Cell Tumor, Childhood Extragonadal Germ Cell Tumor, Eye Cancer, Intraocular Melanoma, Retinoblastoma, Fallopian Tube Cancer, Osteosarcoma, Gallbladder Cancer, Gastric (Stomach) Cancer, Gastrointestinal Carcinoid Tumor, Gastrointestinal Stromal Tumors (GIST) (Soft Tissue Sarcoma), Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Heart Tumors, Childhood Hepatocellular (Liver) Cancer, Histiocytosis, Hodgkin Lymphoma, Hypopharyngeal Cancer (Head and Neck Cancer), Intraocular Melanoma, Islet Cell Tumors, Pancreatic Neuroendocrine Tumors, Kidney (Renal Cell) Cancer, Langerhans Cell Histiocytosis, Laryngeal Cancer, Leukemia, Lip and Oral Cavity Cancer, Liver Cancer, Lung Cancer (e.g., Non-Small Cell, Small Cell, Pleuropulmonary Blastoma, and Tracheobronchial Tumor), Lymphoma, Male Breast Cancer, Malignant Fibrous Histiocytoma of Bone and Osteosarcoma, Melanoma, Intraocular Melanoma, Merkel Cell Carcinoma, Mesothelioma, Metastatic Cancer, Metastatic Squamous Neck Cancer with Occult Primary, Midline Tract Carcinoma, Mouth Cancer, Multiple Endocrine Neoplasia Syndromes, Multiple Myeloma/Plasma Cell Neoplasms, Mycosis Fungoides (Lymphoma), Myelodysplastic Syndromes, Myelodysplastic/Myeloproliferative Neoplasms, Myelogenous Leukemia, Chronic Myeloproliferative Neoplasms, Nasal Cavity and Paranasal Sinus Cancer, Nasopharyngeal Cancer, Neuroblastoma, Non-Hodgkin Lymphoma, Non-Small Cell Lung Cancer, Oral Cancer, Lip and Oral Cavity Cancer and Oropharyngeal Cancer, Osteosarcoma, Undifferentiated Pleomorphic Sarcoma, Ovarian Cancer, Pancreatic Cancer, Pancreatic Neuroendocrine Tumors (Islet Cell Tumors), Papillomatosis (Childhood Laryngeal), Paraganglioma, Paranasal Sinus and Nasal Cavity Cancer, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma (Lung Cancer), Pregnancy and Breast Cancer, Primary Central Nervous System (CNS) Lymphoma, Primary Peritoneal Cancer Prostate Cancer, Rectal Cancer, Recurrent Cancer, Retinoblastoma, Rhabdomyosarcoma, Salivary Gland Cancer, Sarcoma, Childhood Rhabdomyosarcoma, Childhood Vascular Tumors, Soft Tissue Sarcoma, Uterine Sarcoma, Sezary Syndrome, Skin Cancer, Small Cell Lung Cancer Small Intestine Cancer, Soft Tissue Sarcoma, Squamous Cell Carcinoma, Squamous Neck Cancer with Occult Primary, Stomach (Gastric) Cancer, T-Cell Lymphoma, Testicular Cancer, Throat Cancer, Nasopharyngeal Cancer, Oropharyngeal Cancer, Hypopharyngeal Cancer, Thymoma and Thymic Carcinoma, Thyroid Cancer, Tracheobronchial Tumors, Transitional Cell Cancer of the Renal Pelvis and Ureter, Carcinoma of Ureter and Renal Pelvis, Transitional Cell Cancer, Urethral Cancer, Uterine Cancer, Uterine Sarcoma, Vaginal Cancer, Vascular Tumors, Vulvar Cancer, Wilms Tumor, and other Childhood Kidney Tumors.

In some embodiments, the cancer is a multiply drug resistant cancer or a recurrent cancer. It is contemplated that the compositions and methods disclosed here are suitable for both non-metastatic cancers and metastatic cancers. Accordingly, in some embodiments, the cancer is a non-metastatic cancer. In some other embodiments, the cancer is a metastatic cancer. In some embodiments, the composition administered to the subject inhibits metastasis of the cancer in the subject. In some embodiments, the administered composition inhibits tumor growth in the subject.

Accordingly, in one aspect, some embodiments of the disclosure relate to methods for the prevention and/or treatment of a condition in a subject in need thereof, wherein the methods include administering to the subject a composition including one or more of: a SLP-76, CAR, or TCR polypeptide of the disclosure, a recombinant nucleic acid of the disclosure, a recombinant cell of the disclosure, and/or a pharmaceutical composition of the disclosure.

In some embodiments, the administered composition inhibits proliferation of a target cancer cell, and/or inhibits tumor growth of the cancer in the subject. For example, the target cell may be inhibited if its proliferation is reduced, if its pathologic or pathogenic behavior is reduced, if it is destroyed or killed, etc. Inhibition includes a reduction of the measured pathologic or pathogenic behavior of at least about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods include administering to the individual an effective number of the recombinant cells disclosed herein, wherein the recombinant cells inhibit the proliferation of the target cancer cell and/or inhibit tumor growth of a target cancer in the subject compared to the proliferation of the target cell and/or tumor growth of the target cancer in subjects who have not been administered with the recombinant cells. In some embodiments, the target cancer cell is a leukemia cancer cell, a cell derived from a leukemia cancer cell, or a cell in a microenvironment of a leukemia. In some embodiments, the target cancer cell is a neuroblastoma cell, a cell derived from a neuroblastoma cell, or a cell in a microenvironment of a neuroblastoma. In some embodiments, the target cancer cell is an osteosarcoma cell, a cell derived from an osteosarcoma cell, or a cell in a microenvironment of an osteosarcoma.

The terms “administration” and “administering”, as used herein, refer to the delivery of a bioactive composition or formulation by an administration route including, but not limited to, oral, intravenous, intra-arterial, intramuscular, intraperitoneal, subcutaneous, intramuscular, and topical administration, or combinations thereof. The term includes, but is not limited to, administering by a medical professional and self-administering.

Administration of the compositions described herein, e.g., SLP-76, CARs, and/or TCR polypeptides, nucleic acids, recombinant cells, cell cultures, and/or pharmaceutical compositions, can be used in the stimulation of an immune response. In some embodiments, SLP-76, CARs, and/or TCR polypeptides, nucleic acids, recombinant cells, cell cultures, and/or pharmaceutical compositions as described herein are administered to an individual after induction of remission of cancer with chemotherapy, or after autologous or allogeneic hematopoietic stem cell transplantation. In some embodiments, compositions described herein are administered to an individual in need of increasing the production of interferon gamma (IFNγ), TNF-α, and/or interleukin-2 (IL-2) in the treated subject relative to the production of these molecules in subjects who have not been administered one of the therapeutic compositions disclosed herein.

An effective amount of the compositions described herein, e.g., SLP-76, CARs, and/or TCR polypeptides, nucleic acids, recombinant cells, cell cultures, and/or pharmaceutical compositions, is determined based on the intended goal, for example tumor regression. For example, where existing cancer is being treated, the amount of a composition disclosed herein to be administered may be greater than where administration of the composition is for prevention of cancer. One of ordinary skill in the art would be able to determine the amount of a composition to be administered and the frequency of administration in view of this disclosure. The quantity to be administered, both according to number of treatments and dose, also depends on the individual to be treated, the state of the individual, and the protection desired. Precise amounts of the composition also depend on the judgment of the practitioner and are peculiar to each individual. Frequency of administration could range from 1-2 days, to 2-6 hours, to 6-10 hours, to 1-2 weeks or longer depending on the judgment of the practitioner.

In some embodiments, administration is by bolus injection. In some embodiments, administration is by intravenous infusion. In some embodiments, a composition containing recombination cells described herein is administered to a subject in a dosage of about 1 million cells/kg of body weight per day to about 100 million cells/kg of body weight per day. In some embodiments, a composition containing recombination cells as disclosed herein is administered in a dosage of about 0.001 million cells/kg to 100 million cells/kg of body weight per day. In some embodiments, the therapeutic agents are administered in a single administration. In some embodiments, therapeutic agents are administered in multiple administrations, (e.g., once or more per week for one or more weeks).

One of ordinary skill in the art would be familiar with techniques for administering compositions of the disclosure to an individual. Furthermore, one of ordinary skill in the art would be familiar with techniques and pharmaceutical reagents necessary for preparation of these compositions prior to administration to an individual.

In certain embodiments of the present disclosure, the composition of the disclosure contains an aqueous composition that includes one or more of the SLP-76, CARs, and/or TCR polypeptides, nucleic acids, recombinant cells, cell cultures, and/or pharmaceutical compositions as described herein. Aqueous compositions of the present disclosure contain an effective amount of a composition disclosed herein in a pharmaceutically acceptable carrier or aqueous medium. Thus, the “pharmaceutical preparation” or “pharmaceutical composition” of the disclosure can include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the recombinant cells disclosed herein, its use in the manufacture of the pharmaceutical compositions is contemplated. Supplementary active ingredients can also be incorporated into the compositions. For human administration, preparations should meet sterility, pyrogenicity, general safety, and purity standards as required by the FDA Center for Biologics.

One of ordinary skill in the art would appreciate that biological materials should be extensively dialyzed to remove undesired small molecular weight molecules and/or lyophilized for more ready formulation into a desired vehicle, where appropriate. The compositions described herein, e.g., SLP-76, CARs, and/or TCR polypeptides, nucleic acids, recombinant cells, cell cultures, and/or pharmaceutical compositions, can then generally be formulated for administration by any known route, such as parenteral administration. Determination of the amount of compositions to be administered can be made by one of skill in the art, and can in part be dependent on the extent and severity of cancer, and whether the recombinant cells are being administered for treatment of existing cancer or prevention of cancer. The preparation of the compositions containing the SLP-76, CARs, and/or TCR polypeptides, nucleic acids, recombinant cells, cell cultures, and/or pharmaceutical compositions of the disclosure can be known to those of skill in the art in light of the present disclosure.

Upon formulation, the compositions of the disclosure can be administered in a manner compatible with the dosage formulation and in such amount as is therapeutically effective. The compositions can be administered in a variety of dosage forms, such as the type of injectable solutions described above.

In some embodiments, SLP-76, CARs, and/or TCR polypeptides, nucleic acids, recombinant cells, cell cultures, and/or pharmaceutical compositions described herein can be used to overcome T cell exhaustion in the corresponding T cells or in the treated subject relative to a subject who has not been administered one of the therapeutic compositions disclosed herein. In some embodiments, SLP-76, CARs, and/or TCR polypeptides, nucleic acids, recombinant cells, cell cultures, and/or pharmaceutical compositions described herein can be used to stimulate proliferation and/or killing capacity of CAR T-cells in the treated subject relative to the production of these molecules in subjects who have not been administered one of the therapeutic compositions disclosed herein. The production of interferon gamma (IFNγ), tumor necrosis factor-α (TNF-α), and/or interleukin-2 (IL-2) can be stimulated to produce up to about 20 fold, such as any of about 2 fold, 3 fold, 4 fold, 5 fold, 6 fold, 7 fold, 8 fold, 9 fold, 10 fold, 11 fold, 12 fold, 13 fold, 14 fold, 15 fold 16 fold, 17 fold, 18 fold, 19 fold, or 20 fold or higher compared to the production of interferon gamma (IFNγ), tumor necrosis factor-α (TNF-α), and/or interleukin-2 (IL-2) in subjects who have not been administered one of the therapeutic compositions disclosed herein.

### Administration of Recombinant Cells to a Subject

In some embodiments, the methods of the disclosure involve administering an effective amount or number of the recombinant cells provided herein to a subject in need thereof. This administering step can be accomplished using any method of implantation delivery in the art. For example, the recombinant cells can be infused directly in the subject's bloodstream or otherwise administered to the subject.

In some embodiments, the methods disclosed herein include administering, which term is used interchangeably with the terms “introducing,” implanting,” and “transplanting,” recombinant cells into an individual, by a method or route that results in at least partial localization of the introduced cells at a desired site such that a desired effect(s) is/are produced. The recombinant cells or their differentiated progeny can be administered by any appropriate route that results in delivery to a desired location in the individual where at least a portion of the administered cells or components of the cells remain viable. The period of viability of the cells after administration to a subject can be as short as a few hours, e.g., twenty-four hours, to a few days, to as long as several years, or even the lifetime of the individual, i.e., long-term engraftment.

In some embodiments, the delivery of a recombinant cell composition (e.g., a composition including a plurality of recombinant cells according to any of the cells described herein) into a subject by a method or route results in at least partial localization of the cell composition at a desired site. Modes of administration include, e.g., injection, infusion, and instillation. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion. In some embodiments, the route is intravenous. For the delivery of cells, delivery by injection or infusion is a standard mode of administration.

In some embodiments, the recombinant cells are administered systemically, e.g., via infusion or injection.

### Kits

Also provided herein are various kits for the practice of a method described herein. In particular, some embodiments of the disclosure provide kits for the diagnosis of a condition in a subject. Some other embodiments relate to kits for the prevention of a condition in a subject in need thereof. Some other embodiments relate to kits for methods of treating a condition in a subject in need thereof. For example, provided herein, in some embodiments, are kits that include one or more of the SLP-76, CARs, and/or TCR polypeptides, recombinant nucleic acids, engineered cells, or pharmaceutical compositions as provided and described herein, as well as written instructions for making and using the same.

In some embodiments, the kits of the disclosure further include one or more means useful for the administration of any one of the provided SLP-76, CARs, and/or TCR polypeptides, recombinant nucleic acids, engineered cells, or pharmaceutical compositions to an individual. For example, in some embodiments, the kits of the disclosure further include one or more syringes (including pre-filled syringes) and/or catheters (including pre-filled syringes) used to administer any one of the provided SLP-76, CARs, and/or TCR polypeptides, recombinant nucleic acids, engineered cells, or pharmaceutical compositions to an individual. In some embodiments, a kit can have one or more additional therapeutic agents that can be administered simultaneously or sequentially with the other kit components for a desired purpose, e.g., for diagnosing, preventing, or treating a condition in a subject in need thereof.

In some embodiments, a kit can further include instructions for using the components of the kit to practice the methods disclosed herein.

No admission is made that any reference cited herein constitutes prior art. The discussion of the references states what their authors assert, and the inventors reserve the right to challenge the accuracy and pertinence of the cited documents. It can be clearly understood that, although a number of information sources, including scientific journal articles, patent documents, and textbooks, are referred to herein; this reference does not constitute an admission that any of these documents form part of the common general knowledge in the art.

The discussion of the general methods given herein is intended for illustrative purposes only. Other alternative methods and alternatives can be apparent to those of skill in the art upon review of this disclosure, and are to be included within the spirit and purview of this application.

### EXAMPLES

Additional embodiments are disclosed in further detail in the following examples, which are provided by way of illustration and are not in any way intended to limit the scope of this disclosure or the claims.

### Example 1

### Enhancing CAR T Cell Activation by Proximal Signaling Molecules

This Example describes experiments performed to illustrate the function of proximal signaling molecules, when overexpressed, to enhance CAR T cell activation. Exemplary CAR molecules recognizing different extracellular ligands were engineered and tested for their functions to activate cells expressing these molecules and various proximal signaling molecules.

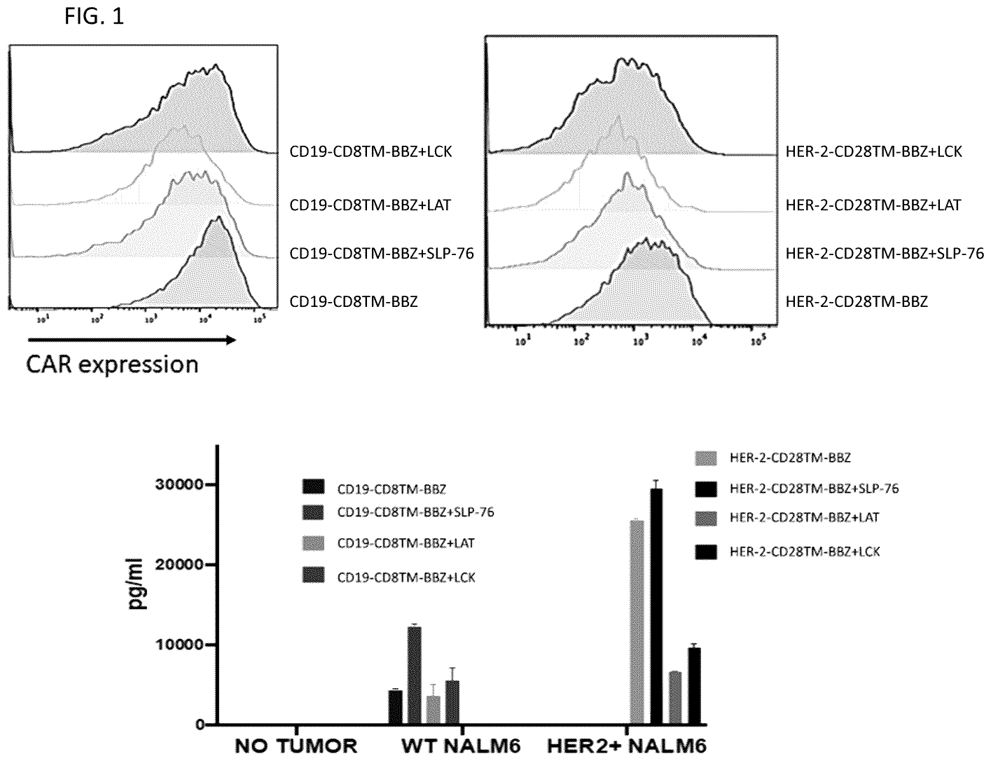

Proximal signaling molecules were tested to see if any of them is sufficient to enhance T cell activation by a CAR molecule. CAR constructs were prepared by linking a scFv (e.g., recognizing CD19 or HER2), a CD8 or CD28 hinge-transmembrane domain (H/TM), and 4-1BB costimulatory domains directly to a CD3zeta domain. The engineered CARs were co-expressed with constructs of proximal signaling molecules in primary T cells. The cytokine production by the T cells in the presence of tumor cells expressing CD19 or HER2 antigens were measured and compared. FIG. 1 shows FACS histograms detecting the extracellular ligand binding domain of each CAR molecule, such as CD19 (the top left panel) or HER2 (the top right panel), representing the expression of the CAR molecule in the T cells. When T cells expressing the CD19 CAR molecules were incubated with wild-type NALM6 cells, which express a high density of CD19, or when T cells expressing the HER2 CAR molecules were incubated with NALM6 cells expressing high density HER2, the CAR molecules activated the corresponding T cells to produce cytokines (e.g., IL-2). As shown in FIG. 1, the bottom panel, such activation (represented by TL-2 production levels) by either of CAR constructs was enhanced only by overexpression of SLP-76 (e.g., SEQ ID NO: 7) in the T cells, but not other proximal signaling molecules, such as LAT (e.g., SEQ ID NO: 8) or LCK (e.g., SEQ ID NO: 9).

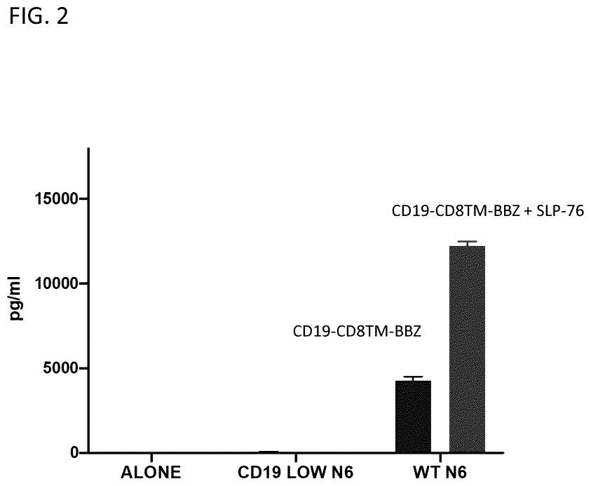

One limitation of current CAR T therapies is that tumors may escape CAR T cells by down-regulating the amount of expressed tumor antigens, usually used as target antigens for recognition by CAR molecules on the CAR T cells. CAR T cell activity may be limited when the antigen density is low. The above co-expression system was then tested under a low antigen density experimental setting. Specifically, T cells expressing CD19 CAR molecules, with or without overexpression of SLP-76 (SEQ ID NO: 7), were incubated with wild-type NALM6 cells or NALM6 cells expressing a low density of CD19 on the cell surface. The activation of T cells was measured by IL-2 production as above. As shown in FIG. 2, SLP-76 (SEQ ID NO: 7; not bound to cell membrane) overexpression enhanced T cell activation through the CD19 CAR molecule in response to only the wild-type NALM6 cells, but not the cells with a low antigen density.

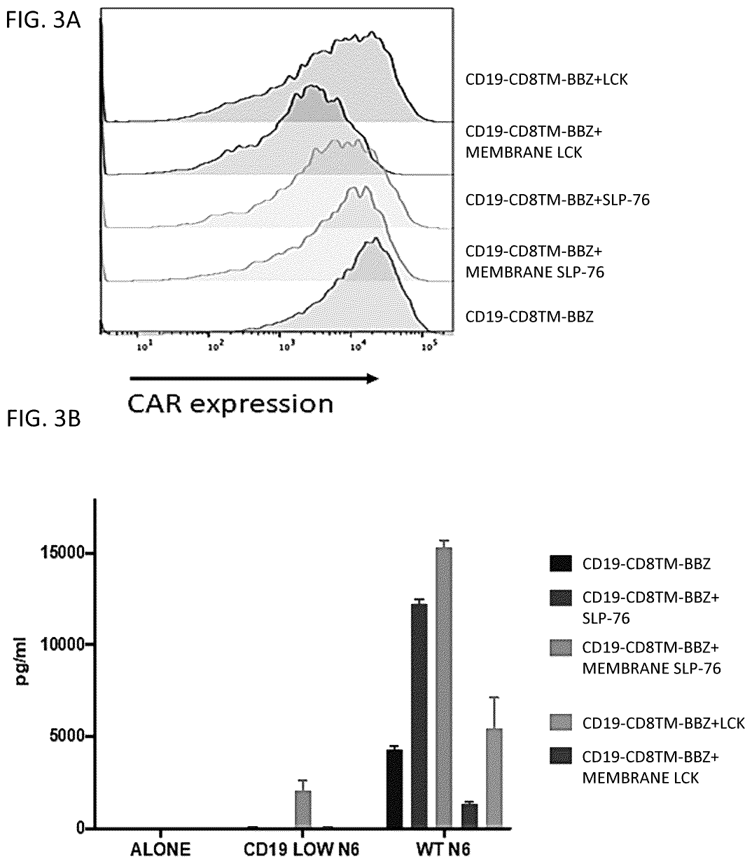

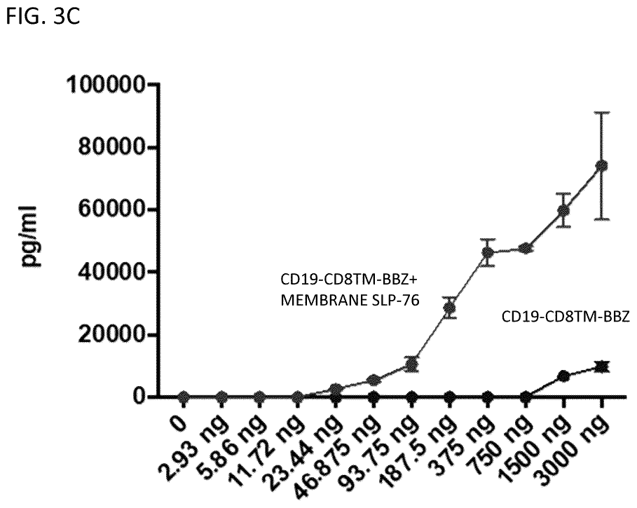

Most proximal signaling molecules are cytosolic and do not have a transmembrane domain. In order to improve the function of SLP-76 to enhance CAR T activity in response to a low antigen density, membrane-bound constructs were prepared and tested. One exemplary construct (VSV-G-CD8TM-SLP-76; SEQ ID NO: 16) contains a CD8 hinge-transmembrane (H/TM) domain conjugated to the N-terminus of SLP-76, plus a short extracellular tag (VSV-G polypeptide) for easy detection by FACS. A similar membrane-bound LCK construct (VSV-G-CD8TM-LCK; SEQ ID NO: 17) was prepared as control. Compared to cytosolic SLP-76 (SEQ ID NO: 7) shown in FIGS. 1 and 2, the exemplary membrane-bound SLP-76 not only further enhanced T cell activity in response to wild-type NALM6 cells (which express a high density of CD19), but was also able to enhance T cell activity in response to a low density of antigens on target NALM6 cells (FIGS. 3A and 3B). Using an anti-idiotype antibody recognizing the extracellular domain of the CD19 CAR to activate or stimulate T cells, a lower amount of such antibodies was sufficient to activate T cells overexpressing the membrane-bound SLP-76 (FIG. 3C). These data show that overexpression of membrane-bound SLP-76 reduces the CAR T cell activation threshold.

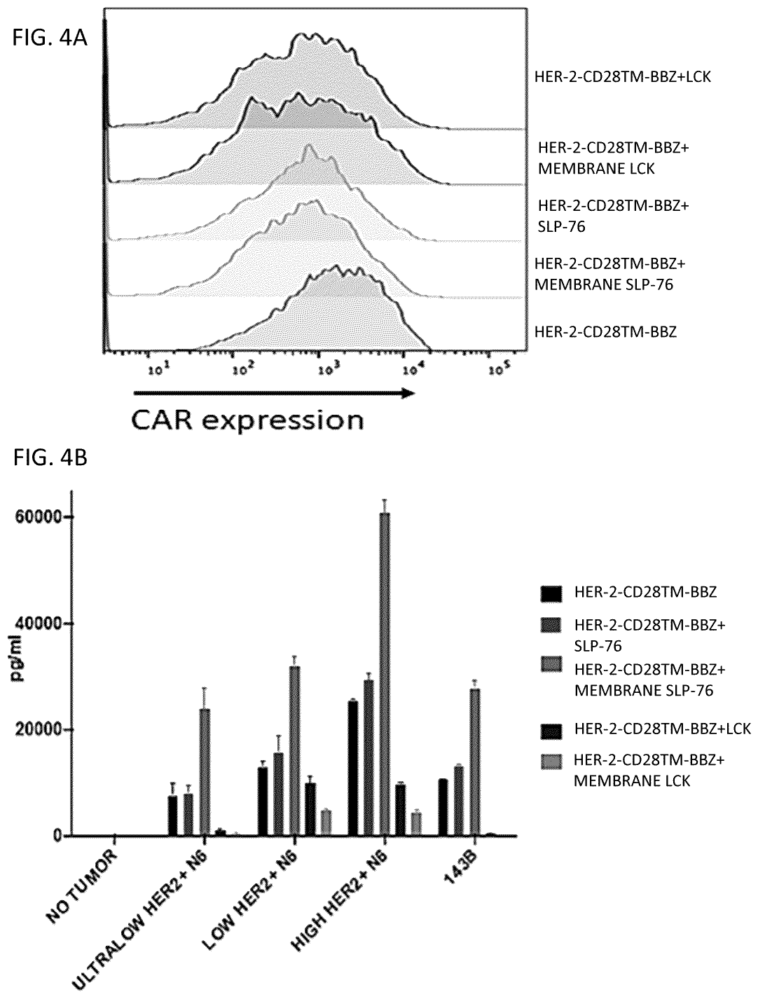

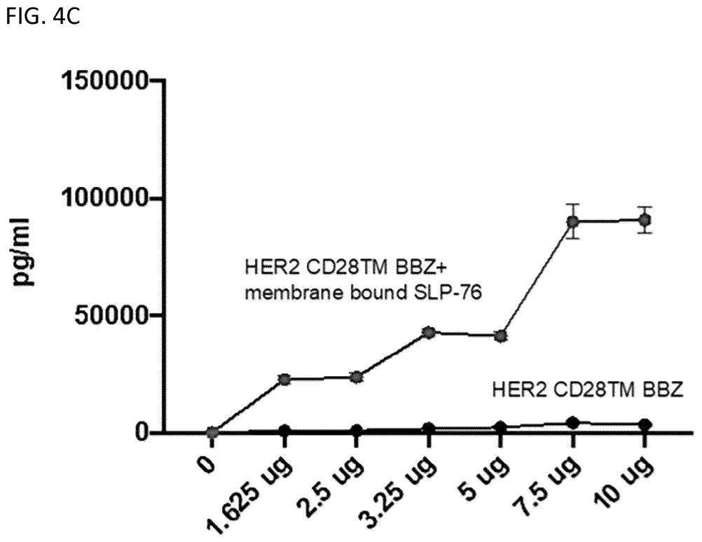

Experiments were also performed using T cells expressing a HER2-targeting CAR molecule (HER2-CD28TM-BBZ). Similarly to FIGS. 3A-3C, the membrane-bound SLP-76 (SEQ ID NO: 16) not only further enhanced T cell activity in response to wild-type NALM6 cells expressing a high level of HER2 or wild-type 143B osteosarcoma cells expressing HER2, but was also able to enhance T cell activity in response to clones of target cells having a low antigen density or an ultra-low antigen density (FIGS. 4A and 4B). A plate was coated with recombinant Her 2 antigens and a lower amount of the coated antigen was sufficient to activate T cells when the membrane-bound SLP-76 was overexpressed (FIG. 4C).

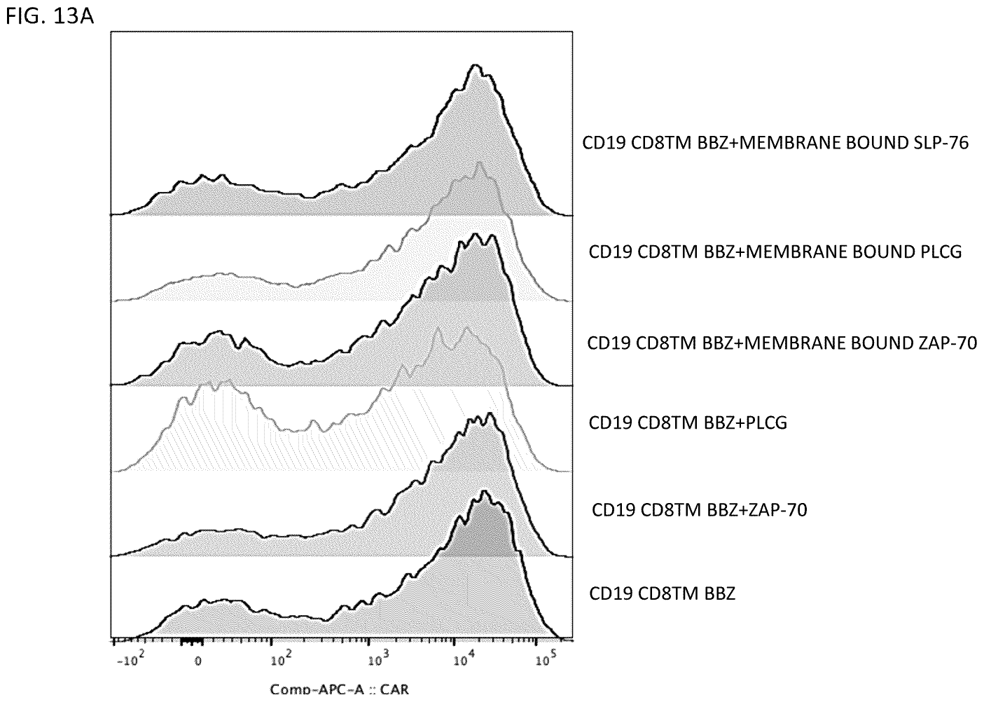

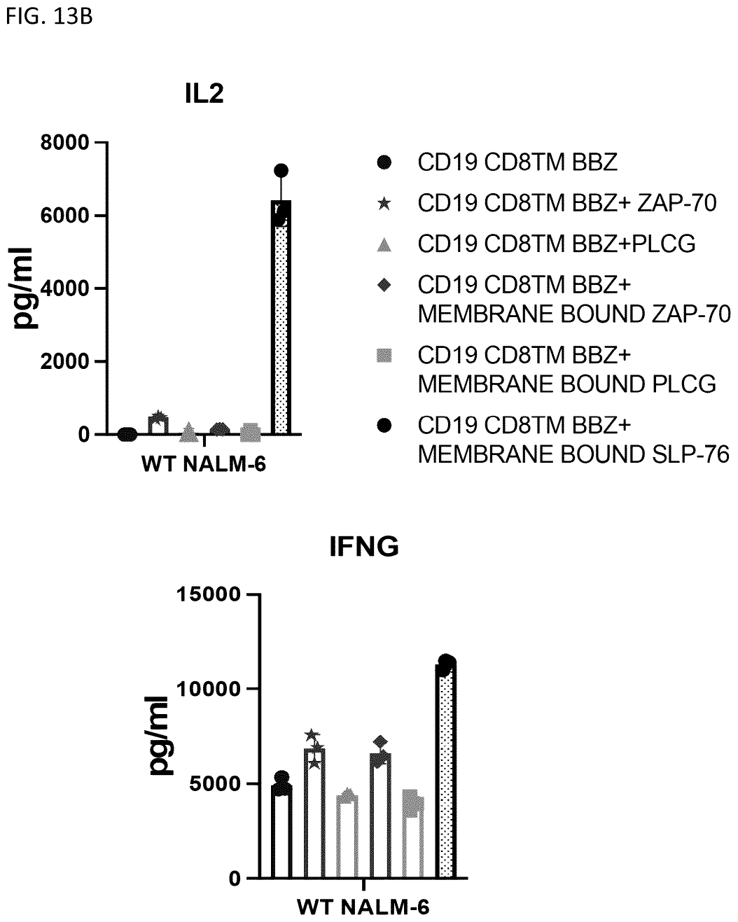

In another experiment, T cells were transduced to overexpress an exemplary CD19-targeting CAR construct (“CD19-CD8TM-BBZ”), with or without a free ZAP-70 construct (ZAP-70-2A-tNGFR, SEQ ID NO: 44), a free PLCγ1 (a.k.a., PLCg1 or PLCG) construct (PLCg1-2A-tNGFR, SEQ ID NO: 48), a membrane-bound ZAP-70 construct [VSV-G-CD8TM-ZAP-70 KIDB (255-600), containing a fragment of ZAP-70 Interdomain B and the kinase domain; SEQ ID NO: 46], a membrane-bound PLCγ1 construct (VSV-G-CD8TM-PLCg1, SEQ ID NO: 50), or the membrane-bound SLP-76 construct described above (SEQ ID NO: 16) (FIG. 13A). As shown in FIG. 13B, only the membrane-bound SLP-76, but not free or membrane-bound ZAP-70 or PLCγ1 constructs, significantly increased cytokine (e.g., IL-2 and IFNγ) production by the T cells expressing the CD19-targeting CAR construct, when contacted with wild-type NALM-6 cells.

### Example 2

### Membrane-Bound SLP-76 Enhances CAR T Activity Independent of SLP-76 or CAR Structure

This Example describes experiments performed to confirm the capability of membrane-bound SLP-76 to enhance CAR T activation through various CAR molecules.

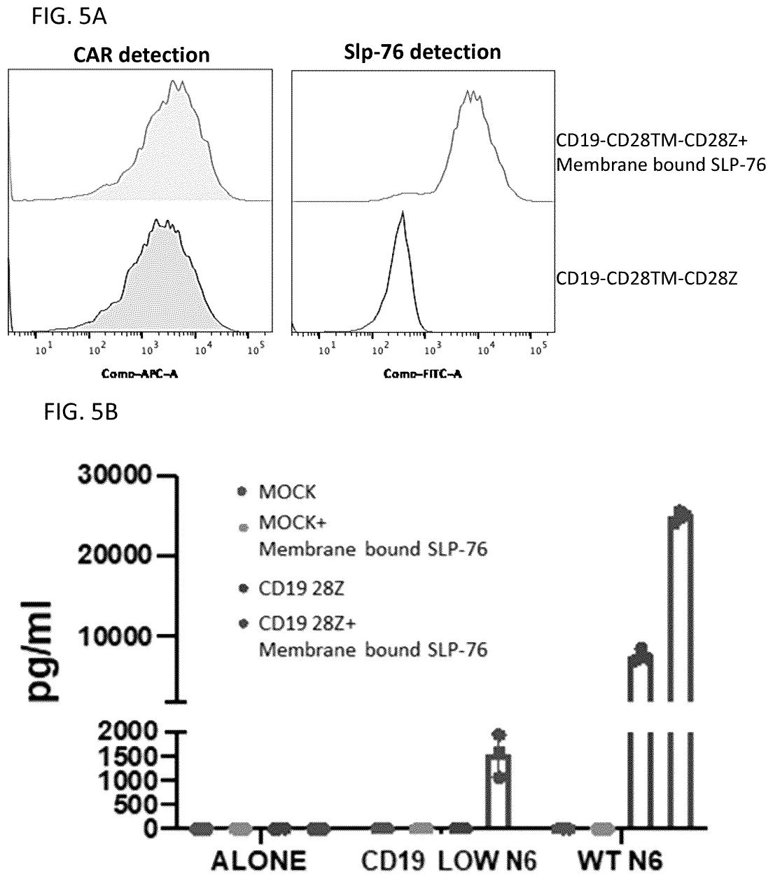

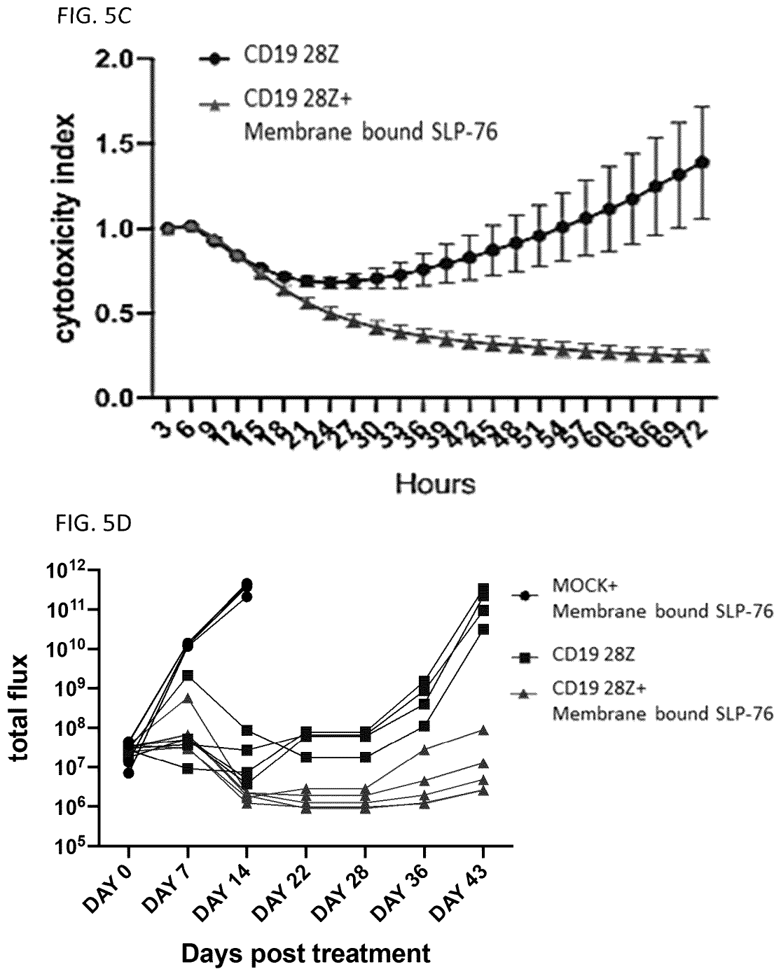

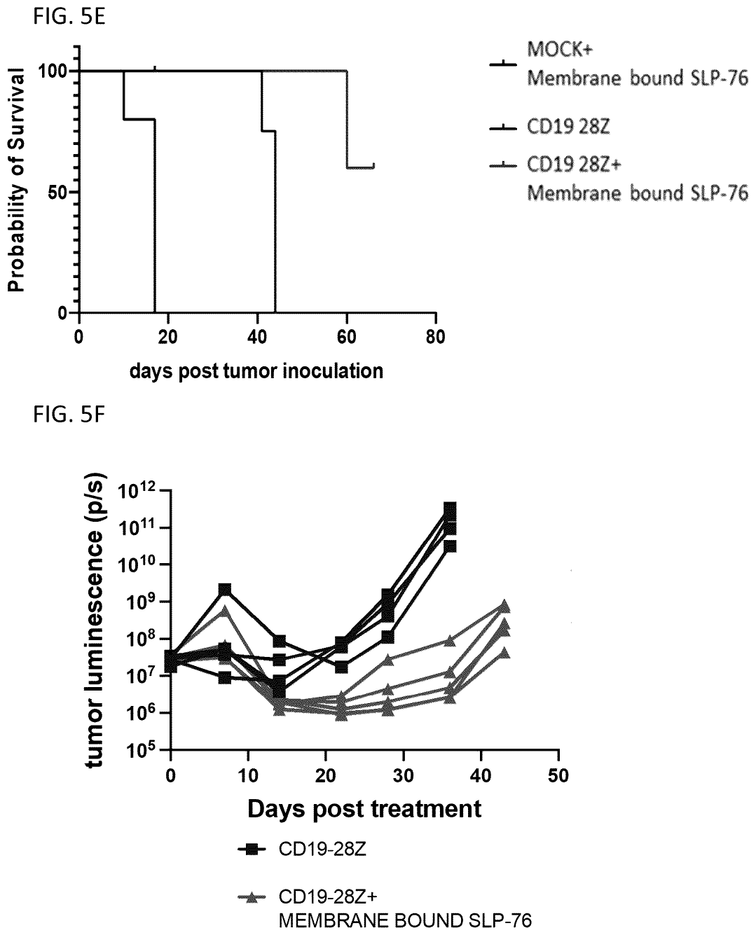

One exemplary CAR construct contains an extracellular domain to recognize CD19, a CD28 transmembrane domain and intracellular region, and a CD3zeta signaling domain (“CD19-CD28TM-CD28Z” or “CD19-28Z”). T cells expressing such CAR construct, with or without overexpressing the membrane-bound SLP-76 construct (SEQ ID NO: 16) (FIG. 5A), were incubated with different tumor cells. IL-2 production levels were measured by ELISA for these T cells after incubation with tumor cells, showing that membrane-bound SLP-76 significantly enhanced T cell activity in response to either a normal antigen density or a low antigen density (FIG. 5B). The cytotoxicity index also confirms such capacity of the membrane-bound SLP-76 to enhance T cell activation to kill tumor cells at low antigen density (FIG. 5C). In an in vivo wild-type NALM6 stress test model, the membrane-bound SLP-76, when expressed in CAR T cells, significantly reduced the tumor burden, especially after 14 days post treatment, compared to the treatment with CAR T cells not expressing the membrane-bound SLP-76 (FIGS. 5D and 5F). Correspondingly, the membrane-bound SLP-76 significantly enhanced the function of CAR T cells to increase animal survival rate (FIG. 5E).

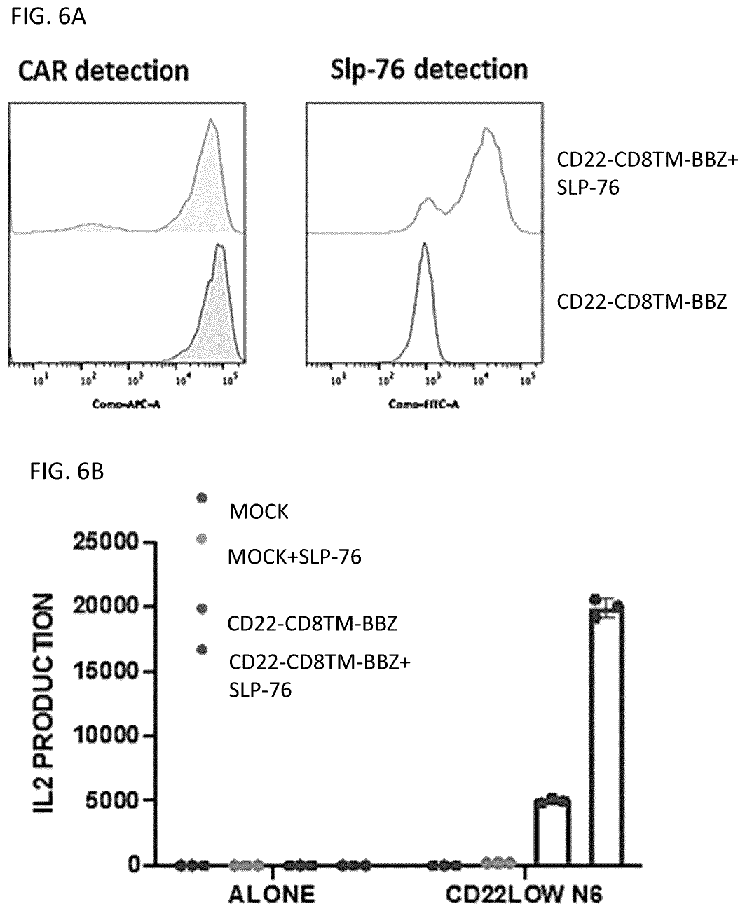

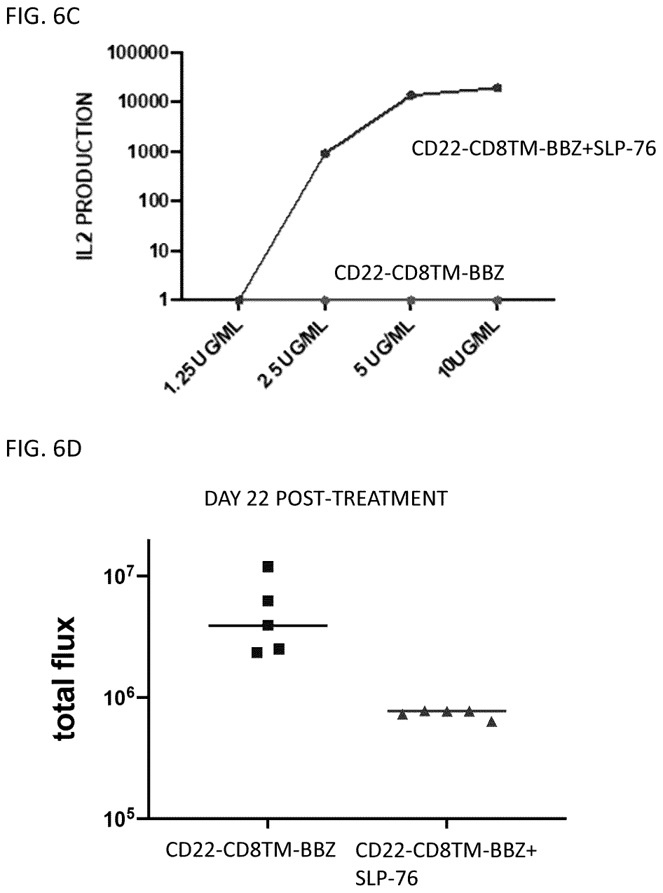

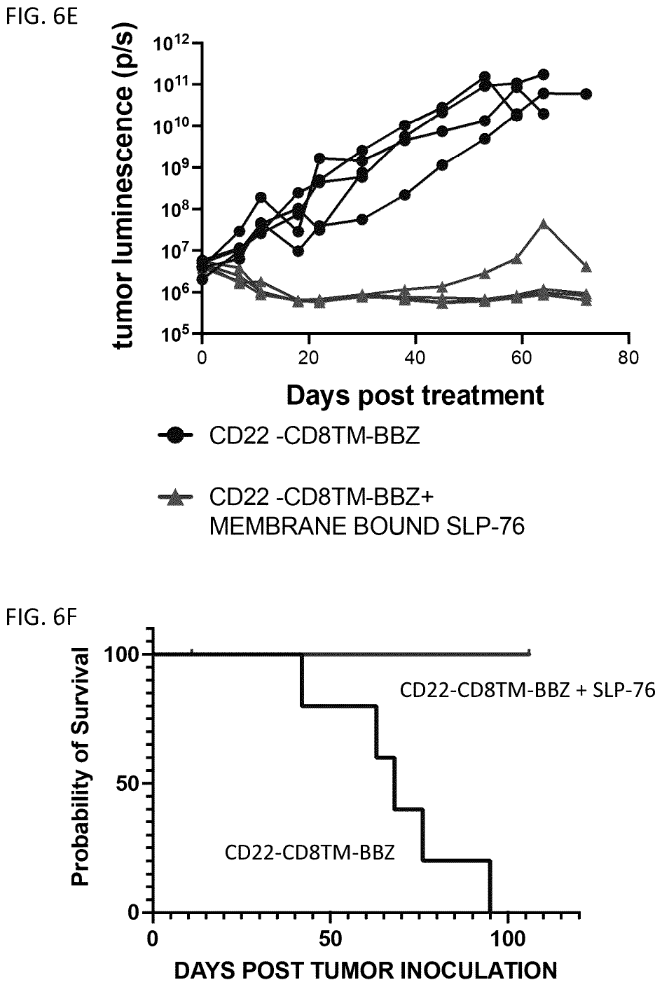

Similar experiments were performed using another exemplary CAR construct containing an extracellular domain recognizing CD22, a CD8 transmembrane domain, a 4-1BB costimulatory domain, and a CD3zeta signaling domain (“CD22-CD8TM-BBZ”). Downregulation of CD22 is a known mechanism of antigen escape from CD22 CAR T cell therapy in patients (Fry et al, Nature Medicine, 2017). T cells expressing such CAR construct, with or without overexpressing the membrane-bound SLP-76 construct (SEQ ID NO: 16) (FIG. 6A), were incubated with tumor cells. IL-2 production levels were measured by ELISA for these T cells after incubation with tumor cells, showing that membrane-bound SLP-76 significantly enhanced T cell activity in response to a low antigen density (FIG. 6B). With a recombinant antigen stimulation assay as described previously, recombinant CD22 was used as the target antigen to activate T cells. A lower amount of such coated antigen was sufficient to activate T cells when membrane-bound SLP-76 was overexpressed (FIG. 6C). In an in vivo CD22LOW NALM6 model, the membrane-bound SLP-76, when expressed on CAR T cells, significantly reduced the tumor burden (e.g., after 22 days post treatment as shown in FIG. 6D), compared to CAR T cells not expressing membrane-bound SLP-76 (FIGS. 6D-6F).

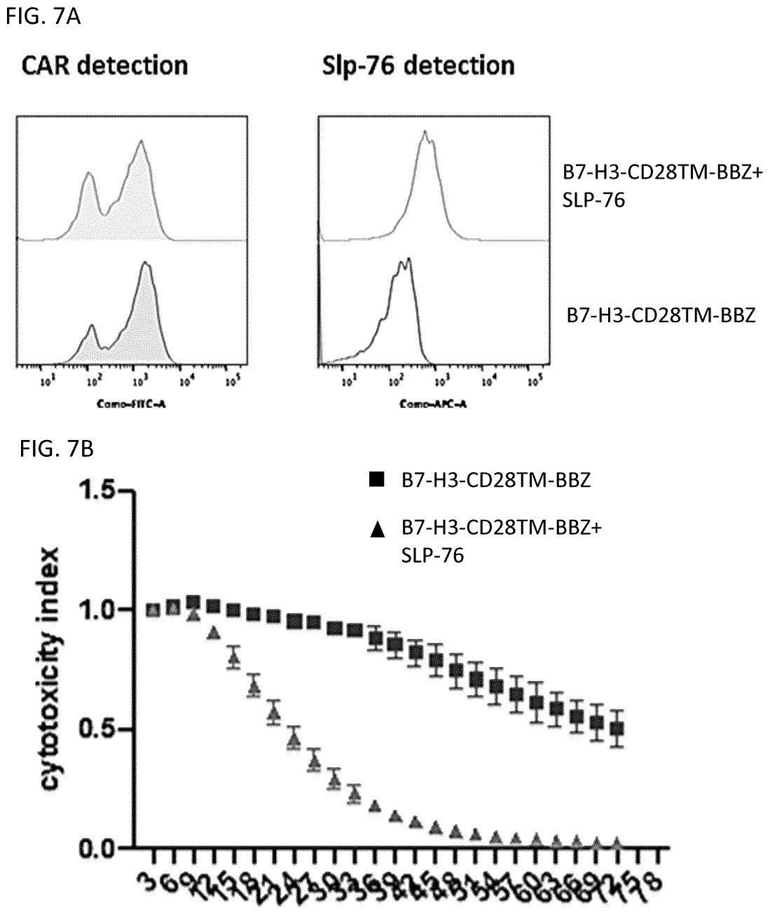

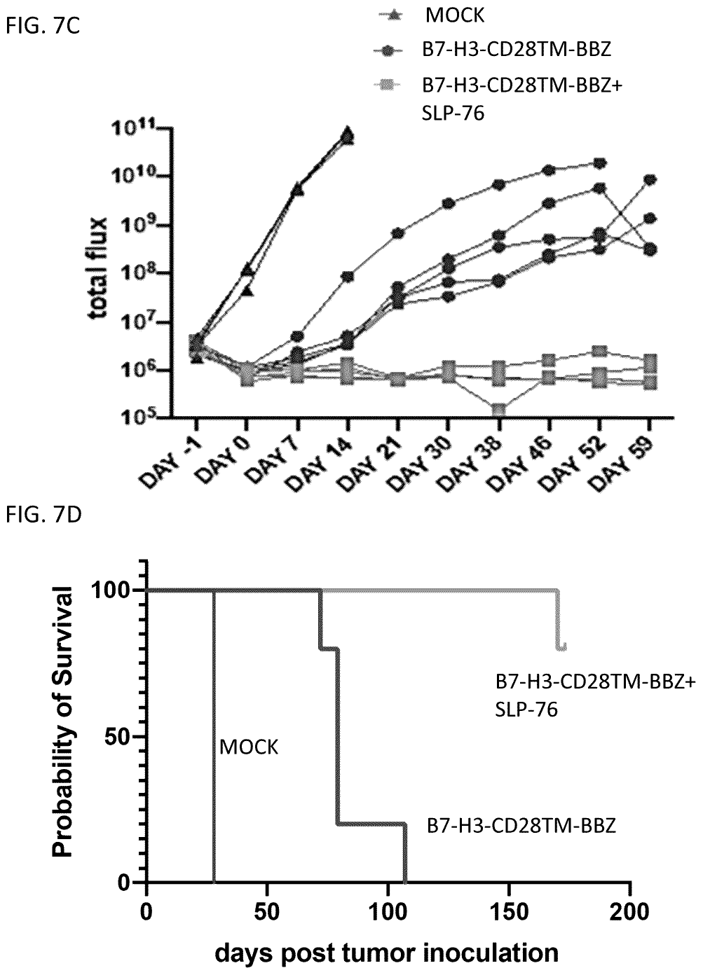

Similar experiments were performed using another exemplary CAR construct containing an extracellular domain recognizing B7-H3, a CD28 transmembrane domain, a 4-1BB intracellular signaling domain, and a CD3zeta signaling domain (“B7-H3-CD28TM-BBZ”). T cells expressing such CAR construct, with or without overexpressing the membrane-bound SLP-76 construct (SEQ ID NO: 16) (FIG. 7A), were incubated with tumor cells. Cytotoxicity index were calculated by detecting alive target tumor cells (neuroblastoma cell line CHLA-255, which expresses a low density of B7-H3) post treatment, showing that membrane-bound SLP-76 significantly enhanced the activity of T cells expressing B7-H3-CD28TM-BBZ to inhibit or kill the target tumor cell (FIG. 7B). In an in vivo CHLA-255 (which expresses a low density of B7-H3) metastatic model, the membrane-bound SLP-76, when expressed on CAR T cells, significantly reduced the tumor burden throughout the time, while CAR T cells not expressing the membrane-bound SLP-76 led to an increase of tumor burden after 7-14 days post treatment (FIG. 7C). As a result, animals treated with CAR T cells overexpressing the membrane-bound SLP-76 survived more than 100 days, much longer than those treated with CAR T cells not expressing the membrane-bound SLP-76 (FIG. 7D).

Thus, the capacity of enhancing CAR T activity of the membrane-bound SLP-76 is not limited by specific CAR constructs.

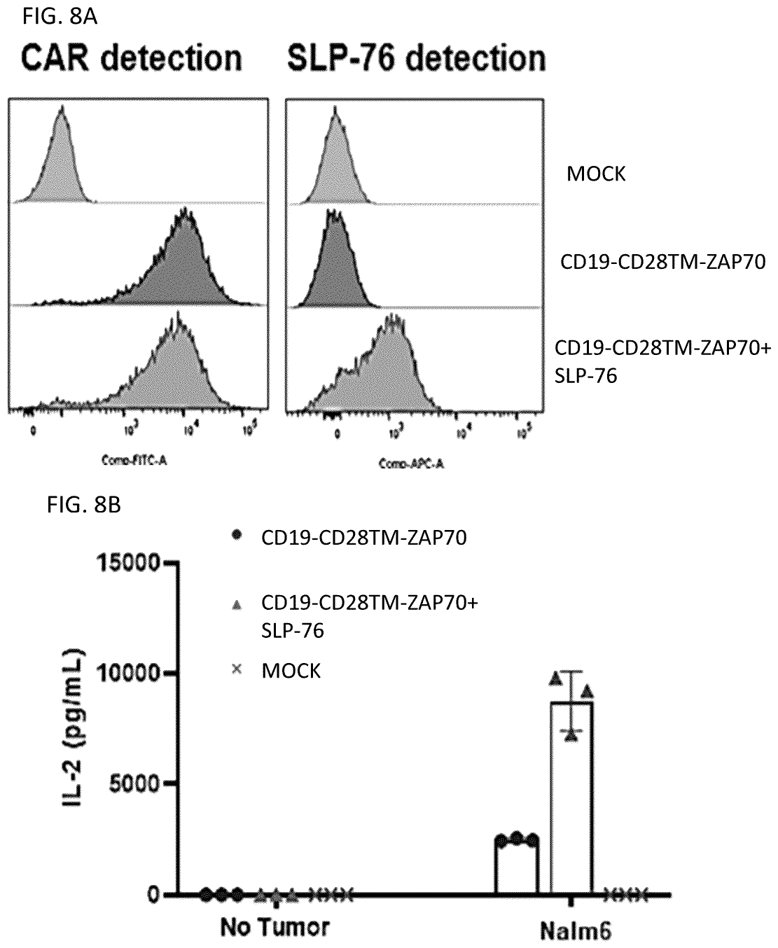

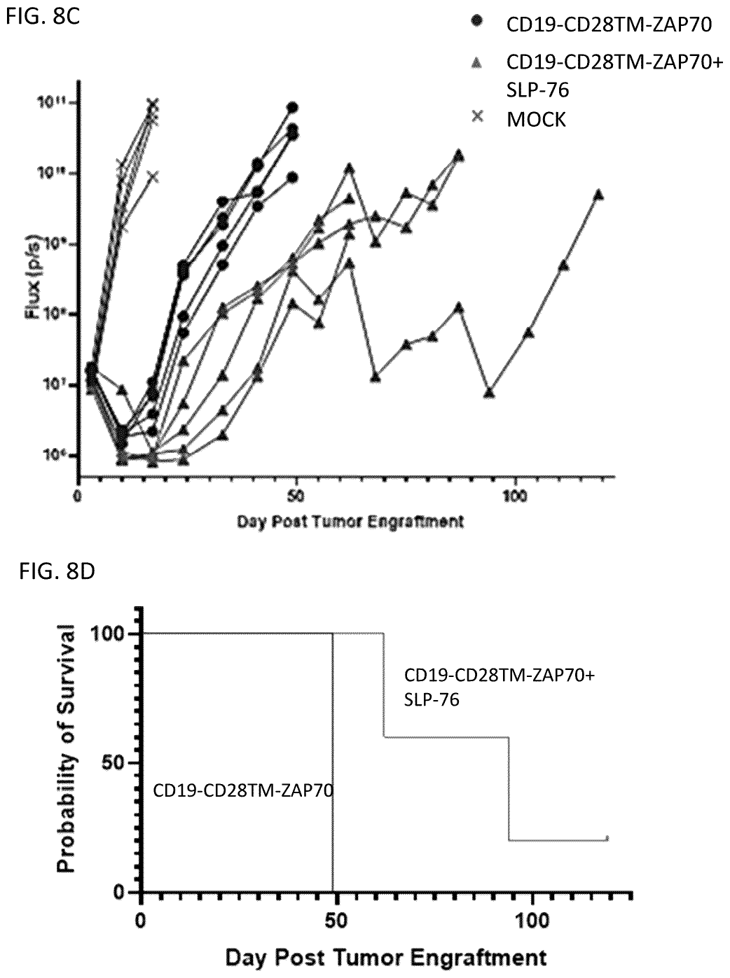

CAR constructs with different proximal signaling molecules were further used to test the function of membrane-bound SLP-76. For example, rather than previously described CAR molecules having a CD3zeta signaling domain, an exemplary CAR construct was produced to contain ZAP70 as the intercellular signaling domain (CD19-CD28TM-ZAP70). T cells expressing such CD19-targeting CAR molecule, with or without overexpressing the membrane-bound SLP-76 construct (SEQ ID NO: 16) (FIG. 8A), were incubated with tumor cells. IL-2 production levels were measured by ELISA for these T cells after incubation with tumor cells, showing that the membrane-bound SLP-76 significantly enhanced T cell activity through the CAR molecule (FIG. 8B). In an in vivo NALM6 xenograft model, the membrane-bound SLP-76, when expressed on CAR T cells, significantly reduced the tumor burden, compared to CAR T cells not expressing the membrane-bound SLP-76 (FIG. 8C). As a result, animals treated with CAR T cells overexpressing the membrane-bound SLP-76 had a higher survival rate than those treated with CAR T cells not expressing the membrane-bound SLP-76 (FIG. 8D).

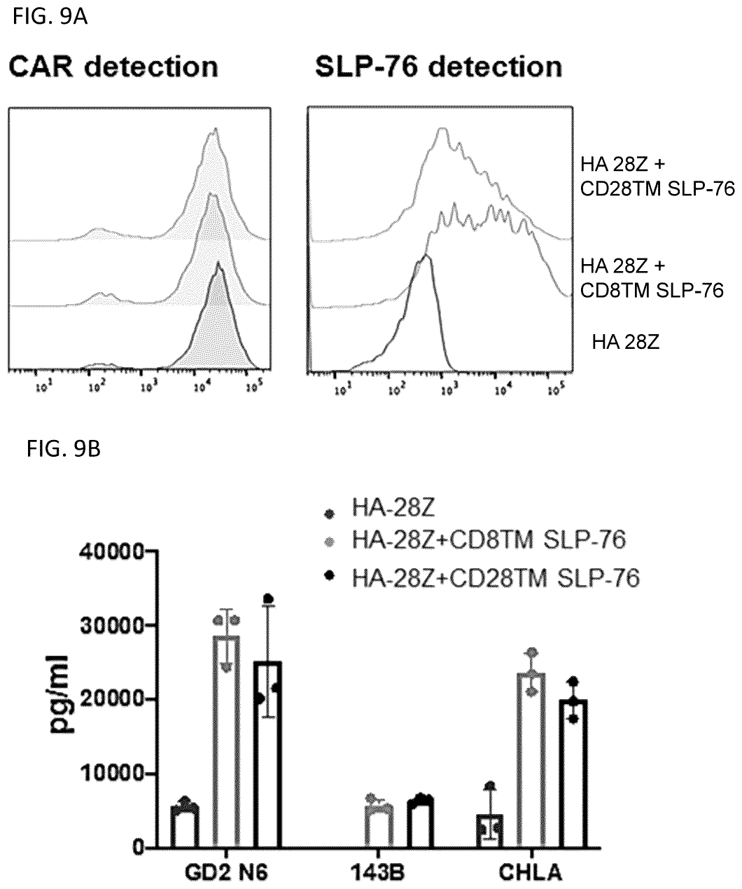

Similar experiments were performed using T cells expressing an exemplary CAR construct containing a high affinity (HA) GD2-targeting extracellular domain, a CD28 transmembrane and costimulatory domain, and a CD3zeta signaling domain (“HA 28Z”), with or without different membrane-bound SLP-76 constructs. For example, an exemplary membrane-bound SLP-76 construct contains a CD28 transmembrane domain (SEQ ID NO: 19; “CD28TM SLP-76”). Another exemplary membrane-bound SLP-76 construct contains a CD8 transmembrane domain (SEQ ID NO: 18; “CD8TM SLP-76”). T cells expressing such high affinity GD2-targeting CAR molecule, with or without overexpressing exemplary membrane-bound SLP-76 constructs (FIG. 9A), were incubated with tumor cells. IL-2 production levels were measured by ELISA for these T cells after incubation with various tumor cells, showing that membrane-bound SLP-76 significantly enhanced CAR T cell activity regardless of the transmembrane domain utilized in the membrane-bound SLP-76 construct (FIG. 9B).

This example demonstrates that membrane-bound SLP-76 enhances the activity of CARs no matter which scFv (antigen specificity), signaling domains (costimulatory or other), or transmembrane domains are contained in the CAR construct or which transmembrane domain is utilized in the membrane-bound SLP-76 construct.

### Example 3

### Further Advantages of Membrane-Bound SLP-76

This Example describes experiments performed to test the effects of membrane-bound SLP-76 to T cell exhaustion and to show its advantages to a reported SLP-76 fusion protein.

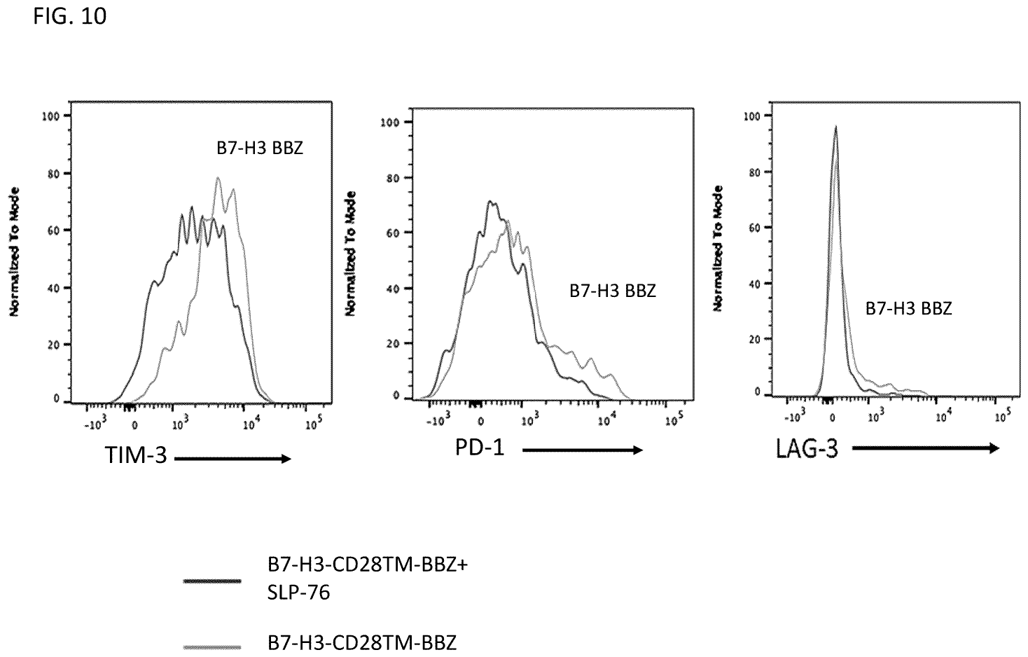

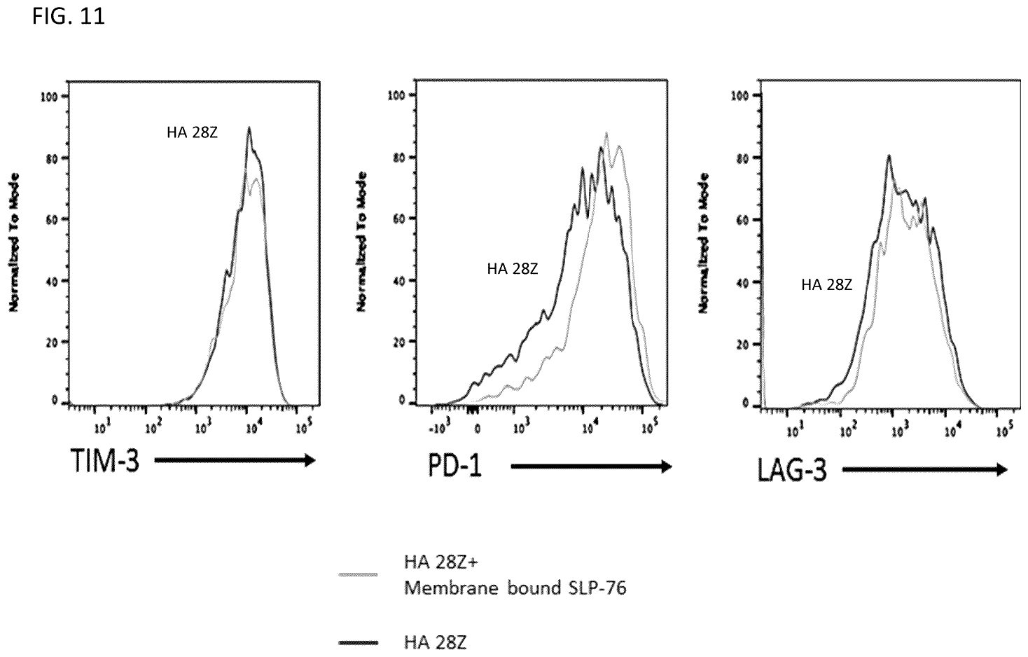

Persistent activation of T cells expressing TCRs or CARs can lead to a gradual development into an exhausted phenotype, usually due to chronic infection or tumor progression. Different CAR constructs described in previous Examples were tested for their effects, when overexpressed, to T cell exhaustion. Specifically, FACS analyses were performed to detect T cell exhaustion biomarkers expressed on the surface of T cells, including, e.g., TIM-3, PD-1, and LAG-3, while the expression levels of such biomarkers are correlated to the degrees of T cell exhaustion. As shown in FIG. 10, overexpression of the B7-H3-CD28TM-BBZ CAR construct, described in previous Examples, induced a moderate expression of all three biomarkers, showing that the B7-H3-CD28TM-BBZ CAR construct may induce a moderate degree of T cell exhaustion. Surprisingly, a membrane-bound SLP-76 (CD19-CD8TM-SLP-76, SEQ ID NO: 40), capable of amplifying CAR T activity and reducing activation threshold, did not exacerbate CAR T cell exhaustion, as co-expression of membrane-bound SLP-76 did not increase biomarker expression levels (FIG. 10). FIG. 11 shows a more severe CAR T exhaustion by overexpressing of the HA 28Z CAR construct. Overexpression of a membrane-bound SLP-76 (Her2-CD8H/TM-SLP-76, SEQ ID NO: 18) in the CAR T cells did not exacerbate cell exhaustion, either (FIG. 11). Thus, regarding the T cell exhaustion issue, membrane-bound SLP-76 is safe to use for boosting other CAR T therapy.

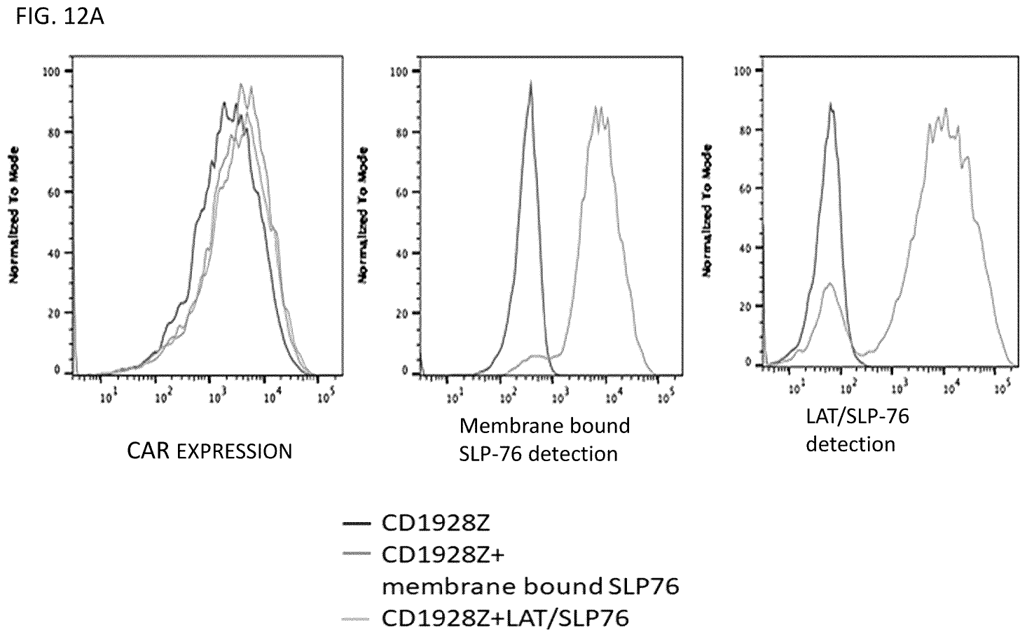

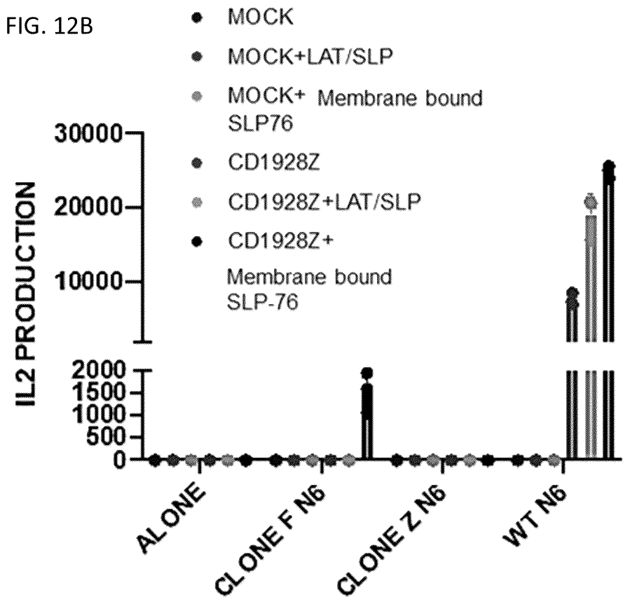

A LAT/SLP-76 chimera protein (LAT transmembrane fused to SLP-76) restores TCR signaling in T cells having the endogenous LAT gene knocked out (Boerth et al. J. Exp. Med. 2000, 192:1047-1058). Such chimera was compared to membrane-bound SLP-76 constructs described in previous Examples. T cells expressing the CD19 28Z CAR construct, described previously, overexpressing a membrane-bound SLP-76 construct (VSV-G-CD8TM-SLP-76, SEQ ID NO: 16) or the LAT/SLP-76 chimera construct (FIG. 12A), were incubated with tumor cells. IL-2 production levels were measured by ELISA for these T cells after incubation with tumor cells, showing that the membrane-bound SLP-76 had a higher capacity than the chimera to enhance T cell activity in response to a high antigen density (FIG. 12B, WT N6). More importantly, the membrane-bound SLP-76, but not the chimera, was able to enhance CAR T activity in response to a low antigen density (FIG. 12B, Clone F N6). Thus, membrane-bound SLP-76 has advantages over the LAT/SLP-76 chimera, especially when the antigen density is low, which is used by tumor cells to escape current T cell therapies with CARs or TCRs.

### Example 4

### Further Studies of Membrane-Bound SLP-76 in Various Cell or Animal Models

This Example describes experiments performed to test the effects of membrane-bound SLP-76 to enhance T cell activation and function in various cell or animal models.

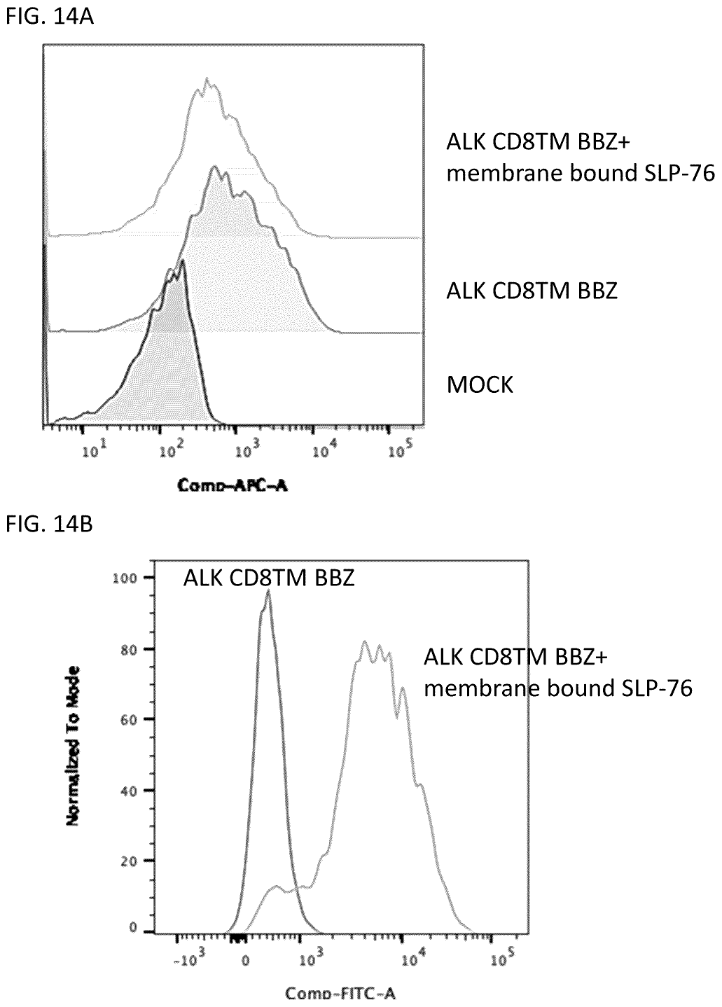

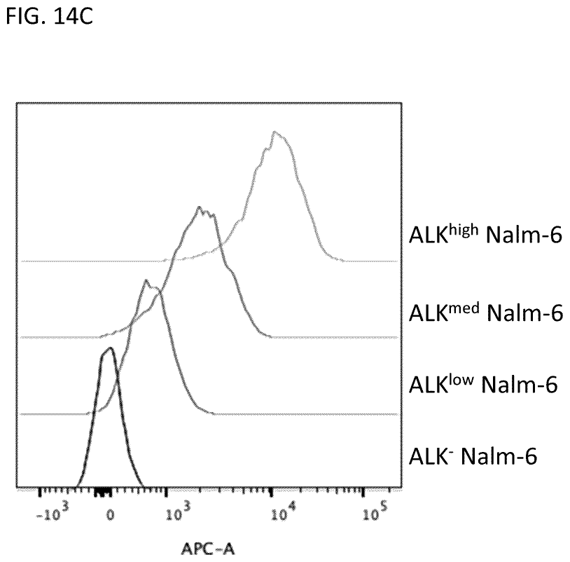

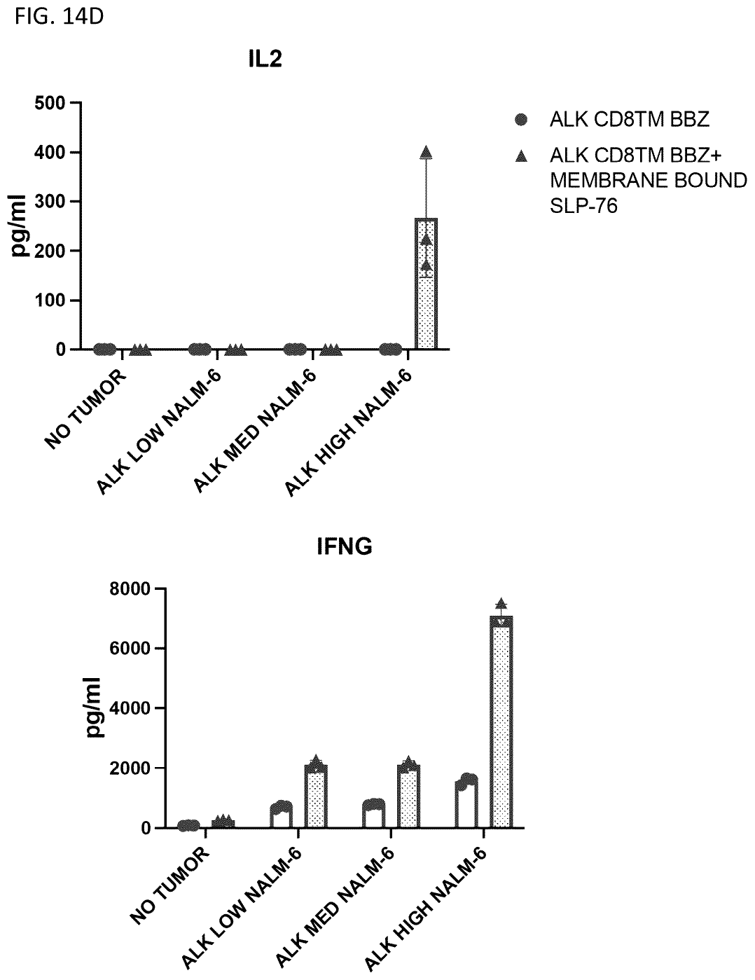

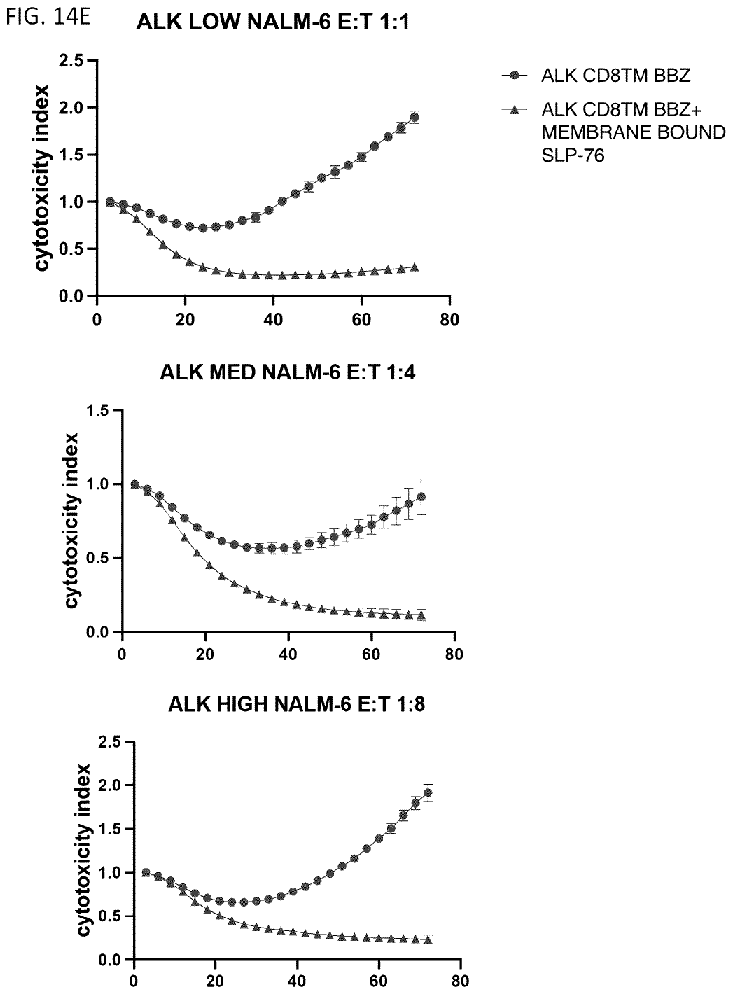

In an exemplary experiment, T cells were transduced to overexpress an Anaplastic Lymphoma Kinase (ALK)-targeting CAR construct (“ALK-CD8TM-BBZ”). This ALK-targeting CAR construct was previously found to express poorly (at low levels) on the surface of T cells, limiting its therapeutic usefulness and its capacity to fully activate T cells (Walker et al, Molecular Therapy, 2017: 25(9):2189-2201; PMID 28676342). ALK-CD8TM-BBZ was expressed on T cells with or without the membrane-bound SLP-76 construct (FIGS. 14A-14B), and then co-cultured with NALM-6 cancer cells expressing different levels of the ALK antigen (FIG. 14C). The membrane-bound SLP-76 construct significantly increased production of both IL-2 and IFNγ by the T cells when contacted with NALM-6 cells expressing a high level of ALK, and significantly increased production of IFNγ, but not IL-2, by the T cells when contacted with NALM-6 cells expressing a medium or low level of ALK (FIG. 14D). The cytotoxicity of the T cells to the target NALM-6 cells was measured. FIG. 14E illustrates that the membrane-bound SLP-76 construct significantly increased the cytotoxicity of the T cells towards all three lines of target NALM-6 cells with different ALK densities.

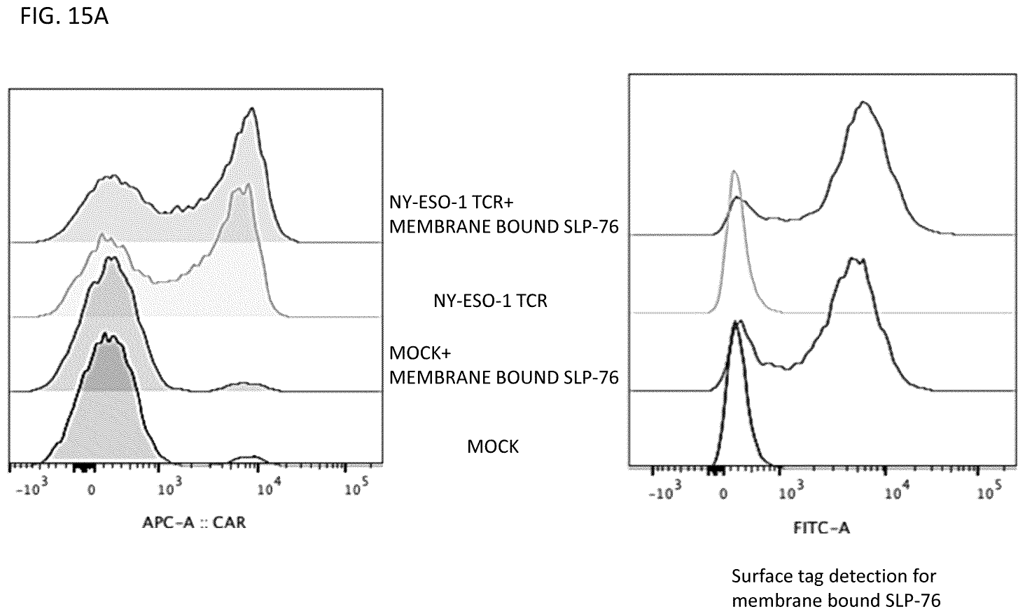

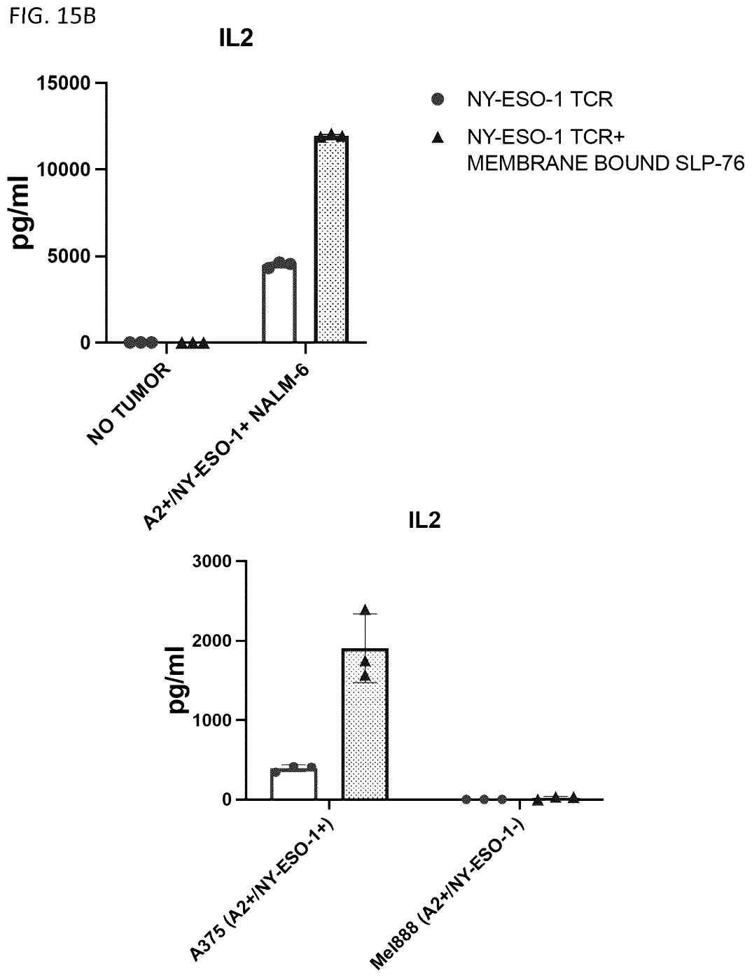

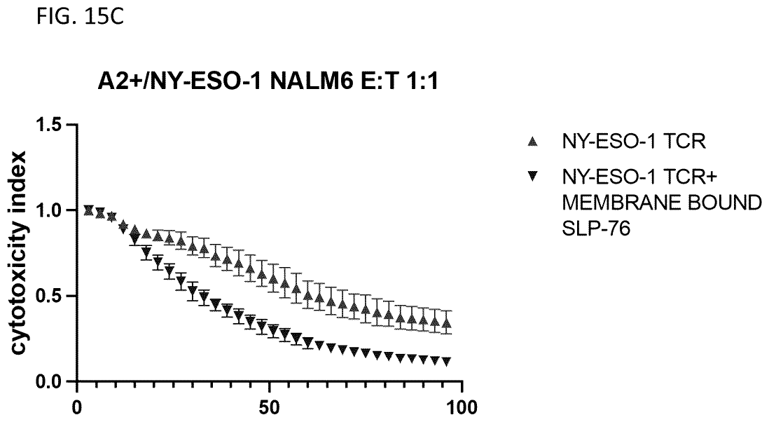

In another exemplary experiment, T cells engineered to express a NY-ESO-1 transgenic TCR construct (A2+/NY-ESO-1) were transduced with or without the membrane-bound SLP-76 construct (FIG. 15A), and co-cultured with either antigen positive tumor cells (A2+/NY-ESO-1+Nalm-6 or A375) or antigen negative tumor cells Mel888. Similarly, the membrane-bound SLP-76 construct significantly increased IL-2 production by the T cells when contacted with the antigen positive NALM-6 or A375 cells but not the antigen negative Mel888 cells (FIG. 15B). The membrane-bound SLP-76 construct also increased the cytotoxicity of the T cells when co-cultured with the antigen positive NALM-6 cells (FIG. 15C).

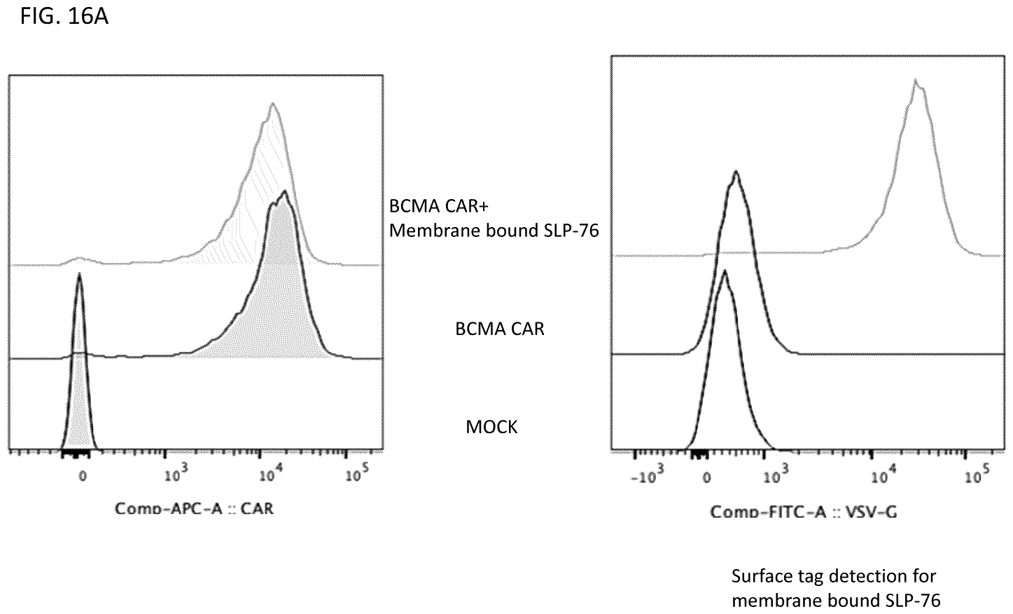

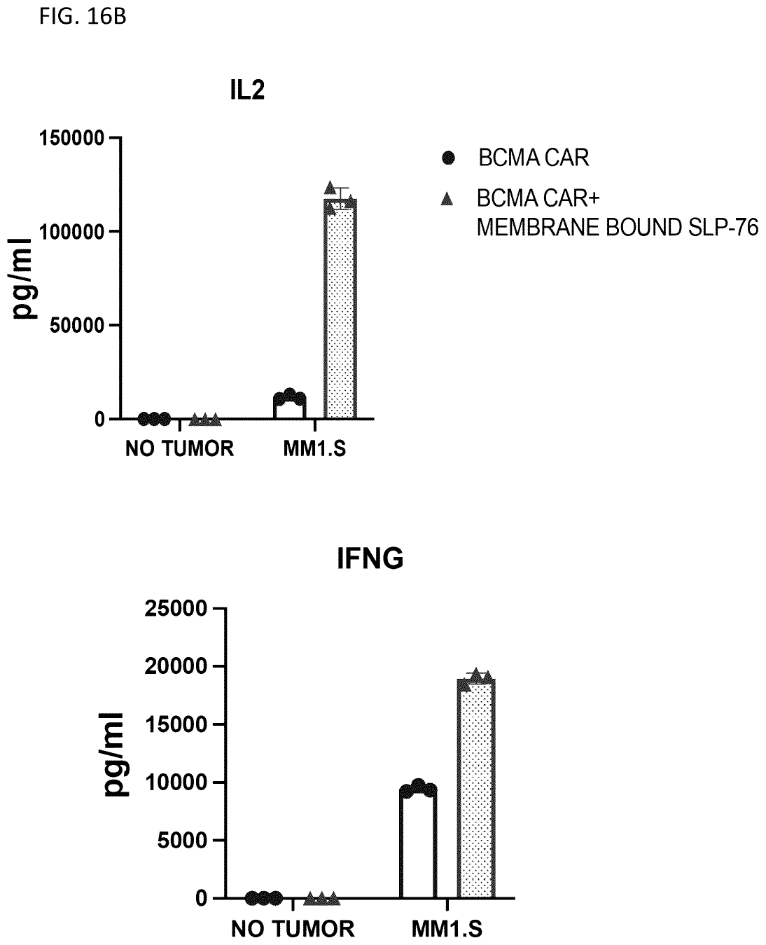

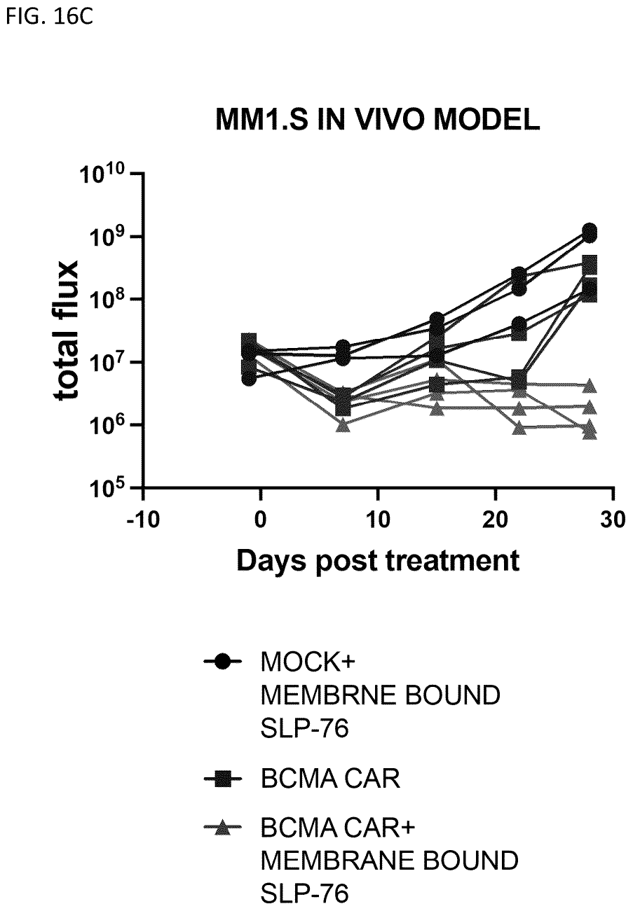

In another exemplary experiment, T cells expressing a B-cell maturation antigen (BCMA)-targeting CAR construct were transduced with or without the membrane-bound SLP-76 construct (FIG. 16A), and co-cultured with MM1.S tumor cells. Similarly, the membrane-bound SLP-76 construct significantly increased cytokine (e.g., IL-2 and IFNγ) production by the T cells (FIG. 16B). In mice inoculated with luciferase-expressing MM1.S tumor cells and treated with the T cells, the membrane-bound SLP-76 construct increased the ability of the T cells to control tumor growth (FIG. 16C).

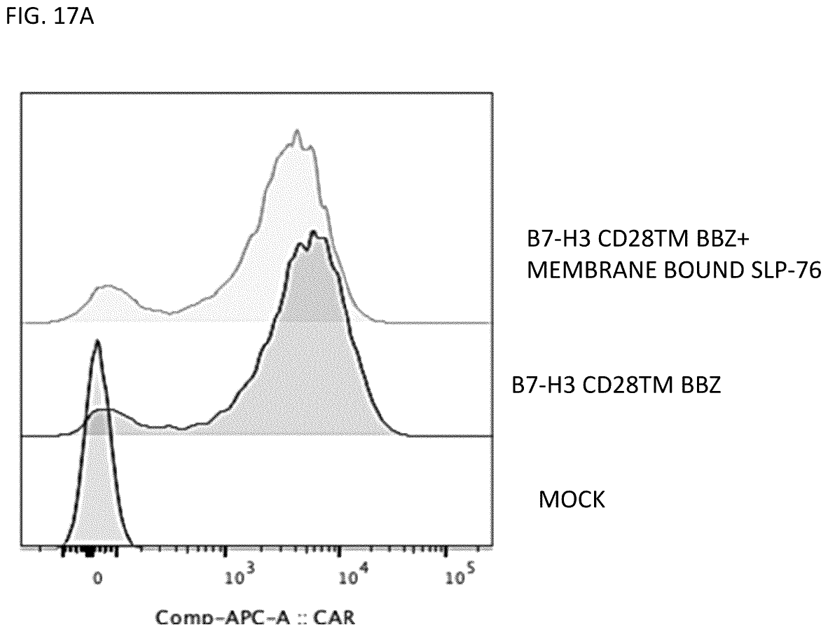

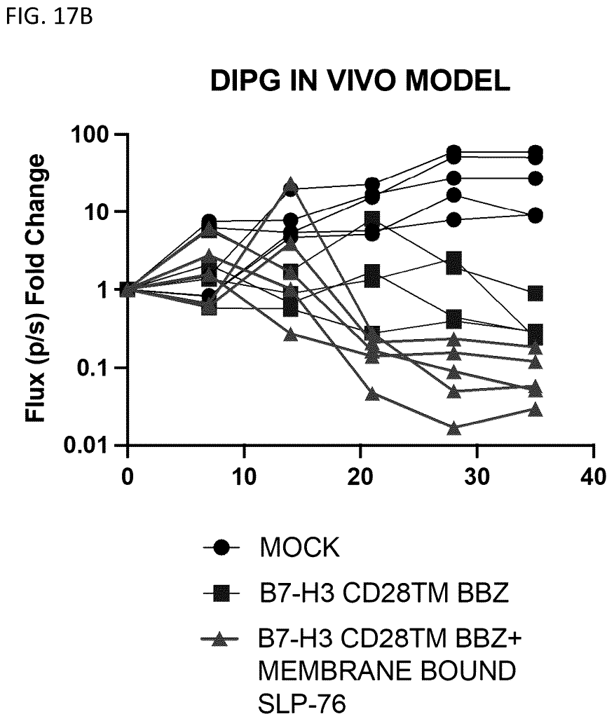

In another exemplary experiment, T cells expressing a B7-H3-targeting CAR construct (“B7-H3-CD28TM-BBZ”) were transduced with or without the membrane-bound SLP-76 construct (FIG. 17A). In mice inoculated with luciferase-expressing diffuse intrinsic pontine glioma 6 xenografts (DIPG-6) and treated with the T cells, the membrane-bound SLP-76 construct increased the ability of the T cells to control tumor growth (FIG. 17B).

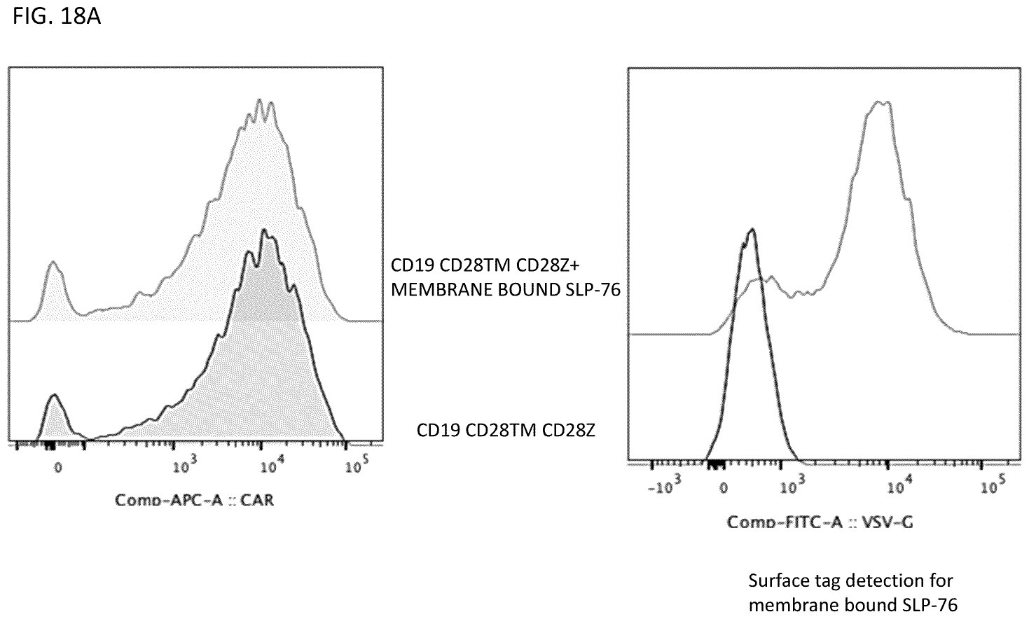

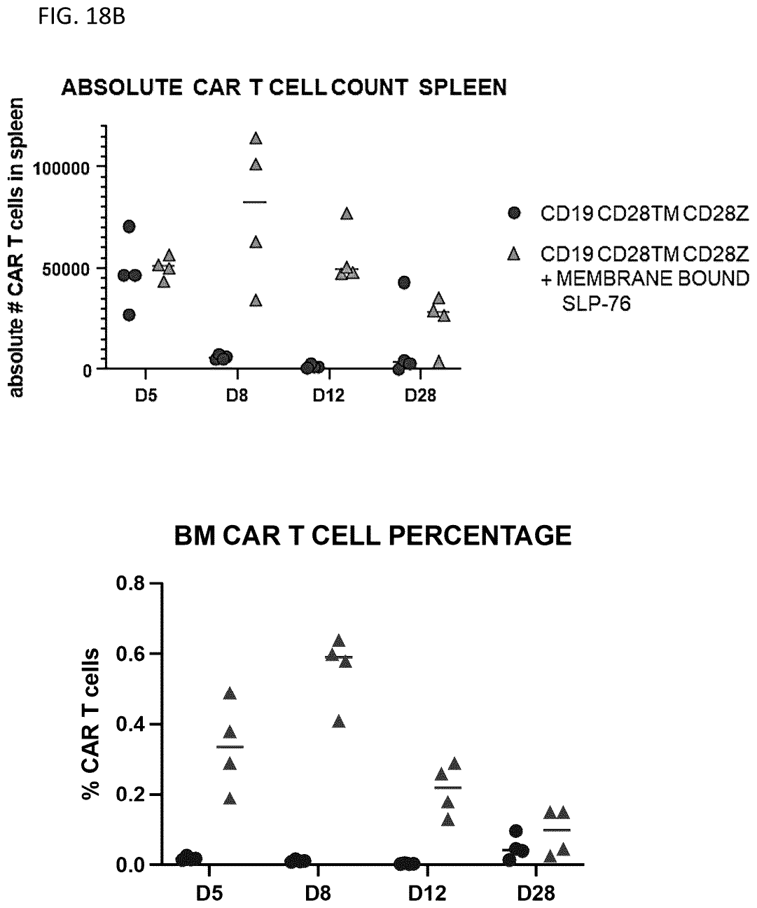

In another exemplary experiment, T cells expressing a CD19-targeting CAR construct (“CD19-CD28TM-CD28Z”) were transduced with or without the membrane-bound SLP-76 construct (FIG. 18A). In a B Cell Acute Lymphoblastic Leukemia (B-ALL) model, mice were inoculated with luciferase-expressing wild-type Nalm-6 cells and treated with the T cells. The membrane-bound SLP-76 construct increased T cell proliferation in either the spleen or bone marrow (FIG. 18B).

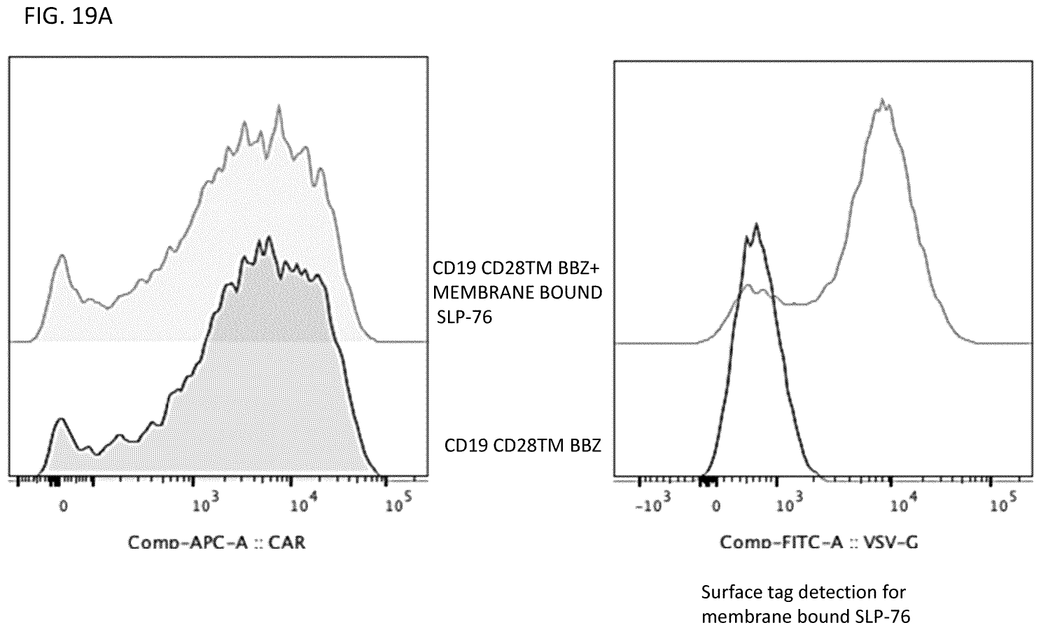

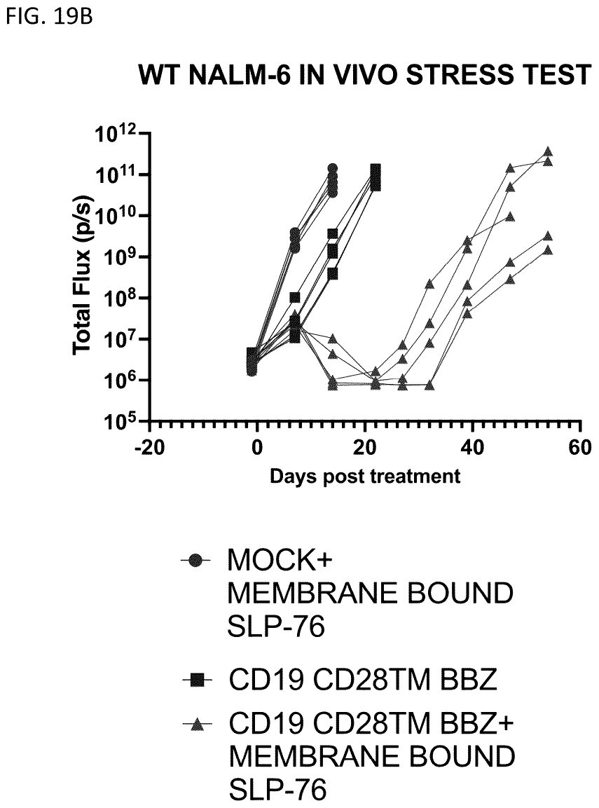

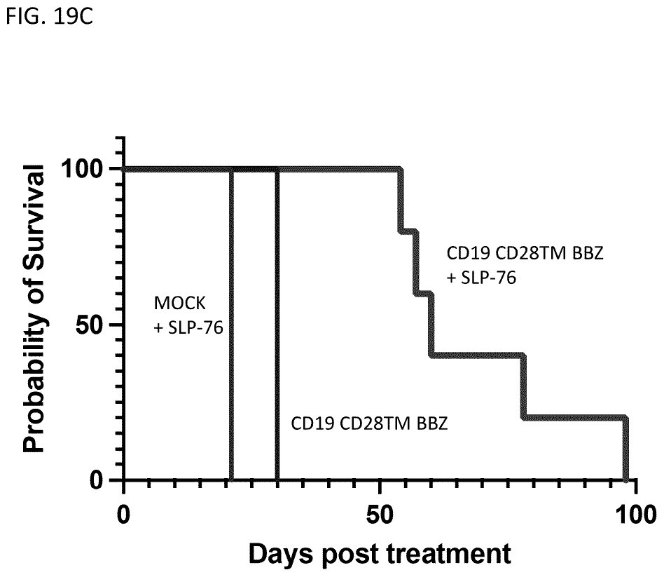

In another exemplary experiment, T cells expressing a CD19-targeting CAR construct (“CD19-CD28TM-BBZ”) were transduced with or without the membrane-bound SLP-76 construct (FIG. 19A). In a stress test model (sub-curative doses of CAR T cells), mice were inoculated with luciferase-expressing leukemia xenografts (WT Nalm-6) and treated with the T cells. The membrane-bound SLP-76 construct increased the ability of the T cells to control tumor growth (FIG. 19B) and to improve mice survival (FIG. 19C).

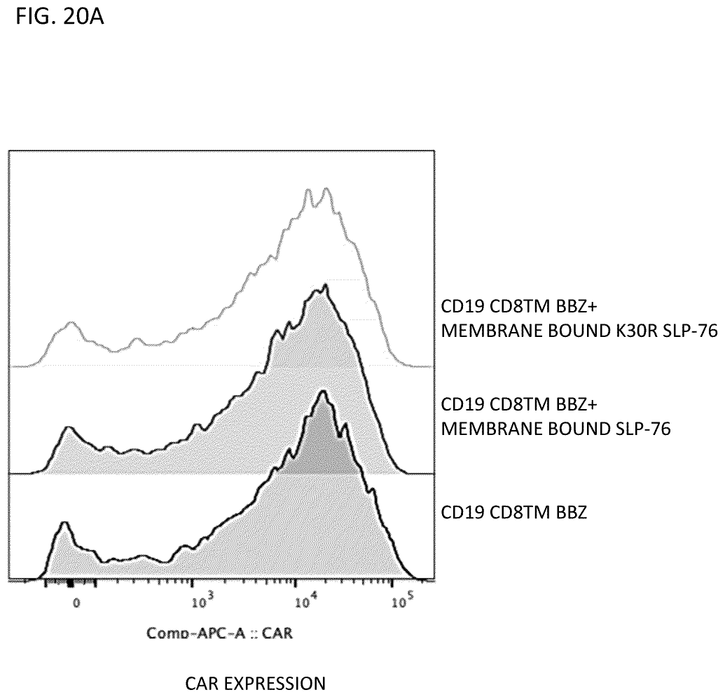

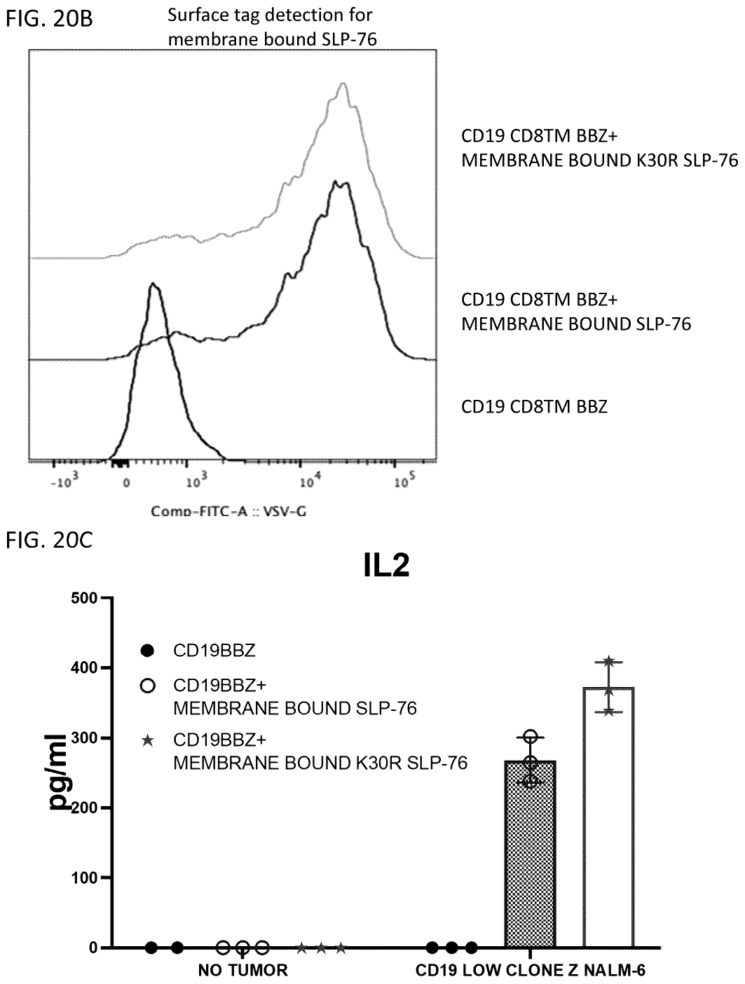

Further experiments were performed to test the function of SLP-76 mutants. In an exemplary experiment, T cells were transduced to express a CD19-targeting CAR construct (“CD19-CD8TM-BBZ”) alone or together with the membrane-bound SLP-76 construct described herein (SEQ ID NO: 16) or a membrane-bound SLP-76 mutation having a K30R substitution (SEQ ID NO: 60) (FIGS. 20A-20B). This K30R mutation was previously reported to reduce ubiquitination downregulation of SLP-76, thus capable of enhancing downstream T cell activation. The resulting T cells were incubated with NALM-6 tumor cells expressing low levels of CD19 (the target antigen), while the levels of IL-2 produced by the T cells were measured. As shown in FIG. 20C, the membrane-bound SLP-76 K30R mutant enhanced T cell activity (shown in IL-2 expression levels) in response to the target antigen on tumor cells, having a higher capacity than the membrane-bound SLP-76 wild-type construct in this scenario with low levels of target antigen on tumor cells.

### Sequence Table

The table below list exemplary sequences for various molecules.

TABLE 1
Informal Sequence Listing

| SEQ | |
|---|---|
| ID | |
| NO | Sequence Identity |
| 1 | SLP-76, with N-terminal methionine, amino acid sequence |
| 2 | LAT, with N-terminal methionine, amino acid sequence |
| 3 | LCK, with N-terminal methionine, amino acid sequence |
| 4 | 2A-tNGFR, variant 1, amino acid sequence |
| 5 | 2A-tNGFR, variant 2, amino acid sequence |
| 6 | 2xHA amino acid sequence |
| 7 | SLP-76-2A-tNGFR, amino acid sequence |
| 8 | LAT-2A-tNGFR, amino acid sequence |
| 9 | LCK-2xHA, amino acid sequence |
| 10 | VSV-G polypeptide, amino acid sequence |
| 11 | ECD recognizing HER2 (scFv), amino acid sequence, |
| | with leader sequence |
| 12 | CD8 Hinge/TM domain, amino acid sequence |
| 13 | CD28 Hinge/TM domain, amino acid sequence |
| 14 | SLP-76 with linker, amino acid sequence |
| 15 | LCK with linker, amino acid sequence |
| 16 | VSV-G-CD8TM-SLP-76, amino acid sequence |
| 17 | VSV-G-CD8TM-LCK, amino acid sequence |
| 18 | HER2-CD8Hinge/TM-SLP-76, amino acid sequence |
| 19 | HER2-CD28Hinge/TM-SLP-76, amino acid sequence |
| 20 | SLP-76, nucleic acid sequence |
| 21 | LAT, nucleic acid sequence |
| 22 | LCK, nucleic acid sequence |
| 23 | 2A-tNGFR, variant 1, nucleic acid sequence |
| 24 | 2A-tNGFR, variant 2, nucleic acid sequence |
| 25 | 2xHA, nucleic acid sequence |
| 26 | SLP-76-2A-tNGFR, nucleic acid sequence |
| 27 | LAT-2A-tNGFR, nucleic acid sequence |
| 28 | LCK-2xHA, nucleic acid sequence |
| 29 | VSV-G polypeptide, nucleic acid sequence |
| 30 | ECD recognizing HER2, with leader |
| | sequence,_nucleic acid sequence |
| 31 | CD8 Hinge/TM domain, nucleic acid sequence |
| 32 | CD28 Hinge/TM domain, nucleic acid sequence |
| 33 | SLP-76 with linker, nucleic acid sequence |
| 34 | LCK with linker, nucleic acid sequence |
| 35 | VSV-G-CD8TM-SLP-76, nucleic acid sequence |
| 36 | VSV-G-CD8TM-LCK, nucleic acid sequence |
| 37 | HER2-CD8Hinge/TM-SLP-76, nucleic acid sequence |
| 38 | HER2-CD28Hinge/TM-SLP-76, nucleic acid sequence |
| 39 | ECD recognizing CD19 (scFv), amino acid sequence, |
| | with leader sequence |
| 40 | CD19-CD8TM-SLP-76, amino acid sequence |
| 41 | ECD recognizing CD19 (scFv), nucleic acid sequence |
| 42 | CD19-CD8TM-SLP-76, nucleic acid sequence |
| 43 | ZAP-70 for preparing the free ZAP-70 construct, |
| | amino acid sequence |
| 44 | ZAP-70-2A-tNGFR (free ZAP-70 construct), amino acid |
| | sequence (SEQ ID NO: 43 + SEQ ID NO: 4) |
| 45 | ZAP-70 KIDB (255-600) for preparing the membrane-bound |
| | ZAP-70 construct, amino acid sequence |
| 46 | VSV-G-CD8TM-ZAP-70 KIDB (255-600) (the membrane-bound |
| | ZAP-70 construct), amino acid sequence (SEQ ID NO: |
| | 10 + SEQ ID NO: 12 + SEQ ID NO: 45) |
| 47 | PLCγ1 for preparing the free PLCγ1 construct, |
| | amino acid sequence |
| 48 | PLCγ1-2A-NGFR (the free PLCγ1 construct), |
| | amino acid sequence (SEQ ID NO: 47 + |
| | SEQ ID NO: 4) |
| 49 | PLCγ1 for preparing the membrane-bound PLCγ1 construct, |
| | with a N-terminal linker, amino acid sequence |
| 50 | VSV-G-CD8TM-PLCγ1 (the membrane-bound PLCγ1 construct), |
| | amino acid sequence (SEQ ID NO: 10 + SEQ ID NO: |
| | 12 + SEQ ID NO: 49) |
| 51 | ZAP-70 for preparing the free ZAP-70 construct, |
| | nucleic acid sequence |
| 52 | ZAP-70-2A-tNGFR (the free ZAP-70 construct), |
| | nucleic acid sequence |
| 53 | ZAP-70 KIDB (255-600) for preparing the membrane-bound |
| | ZAP-70 construct, nucleic acid sequence |
| 54 | VSV-G-CD8TM-ZAP-70 KIDB (255-600) (the membrane-bound |
| | ZAP-70 construct), nucleic acid sequence |
| | (SEQ ID NO: 29 + SEQ ID NO: 31 + SEQ ID NO: 53) |
| 55 | PLCγ1 for preparing the free PLCγ1 construct, |
| | nucleic acid sequence |
| 56 | PLCγ1-2A-NGFR (the free PLCγ1 construct), nucleic |
| | acid sequence |
| 57 | PLCγ1 for preparing the membrane-bound PLCγ1 construct, |
| | with a N-terminal linker, nucleic acid sequence |
| 58 | VSV-G-CD8TM-PLCγ1 (the membrane-bound PLCγ1 construct), |
| | nucleic acid sequence |
| 59 | SLP-76 K30R with linker, amino acid sequence |
| 60 | VSV-G-CD8TM-SLP-76 K30R, amino acid sequence |
| | (SEQ ID NO: 10 + SEQ ID NO: 12 + SEQ ID NO: 59) |
| 61 | SLP-76 K30R with linker, nucleic acid sequence |
| 62 | VSV-G-CD8TM-SLP-76 K30R, nucleic acid sequence |
| | (SEQ ID NO: 29 + SEQ ID NO: 31 + SEQ ID NO: 61) |

What is claimed is:

1. A composition comprising an isolated recombinant cell modified to overexpress and/or contain elevated levels of a SLP-76 polypeptide, wherein the recombinant cell is capable of being activated by a target antigen expressed at a low density.

2. The composition of claim 1, wherein the isolated recombinant cell further comprises a T-cell receptor (TCR) polypeptide and/or a chimeric antigen receptor (CAR) polypeptide, wherein the TCR or the CAR polypeptide has a binding affinity for the target antigen or for an adaptor molecule specifically recognizing the target antigen.

3. The composition of claim 2, wherein the isolated recombinant cell comprises a TCR polypeptide, and wherein the target antigen is expressed by a target cell and presented to the TCR polypeptide by an antigen-presenting cell (APC).

4. The composition of claim 2 or 3, wherein the isolated recombinant cell comprises an endogenous TCR polypeptide.

5. The composition of claim 2, wherein the isolated recombinant cell comprises a CAR polypeptide, and wherein the target antigen is expressed by a target cell.

6. The composition of claim 3 or 5, wherein the target cell is a cell correlated to a proliferative disease, a hematological malignancy, a solid tumor, an autoimmune disease, an inflammation, an allergic disease, an infection, and/or a senescence/aging.

7. The composition of claim 6, wherein the target cell is a cancer cell.

8. The composition of claim 5, wherein the CAR polypeptide comprises
a) an extracellular ligand-binding domain having a binding affinity for the target antigen or for an adaptor molecule specifically recognizing the target antigen;
b) a transmembrane domain;
c) an intracellular signaling domain comprising a proximal signaling molecule; and
d) optionally, a hinge domain and/or a costimulatory domain.

9. The composition of claim 8, wherein the intracellular signaling domain of the CAR polypeptide comprises a full-length or biologically active fragment of a protein kinase, a G protein, a GTP-binding protein, an adaptor signaling protein, or a scaffold protein capable of inducing host cell activation.

10. The composition of claim 9, wherein the intracellular signaling domain comprises CD3ζ, CD3-epsilon, CD3-gamma, DAP12, ZAP70, PLCG1, PKC, ITK, NCK, VAV1, GRB2, GADS, SOS1, ADAP, SYK, LYN, PI3K, or BLNK, or a biologically active fragment, mutant, or variant thereof.

11. The composition of any one of claims 2 to 10, wherein the TCR polypeptide and/or the CAR polypeptide induce exhaustion of the recombinant cell.

12. The composition of claim 11, wherein overexpression of the SLP-76 polypeptide does not enhance exhaustion of the recombinant cell.

13. The composition of any one of claims 2 to 12, wherein the isolated recombinant cell further expresses at a low density of the TCR polypeptide and/or the CAR polypeptide.

14. The composition of any one of claims 1 to 13, wherein the SLP-76 polypeptide is bound to the recombinant cell membrane.

15. The composition of claim 14, wherein the SLP-76 polypeptide is bound to the cell membrane via a transmembrane domain.

16. The composition of claim 14, wherein the SLP-76 polypeptide is bound to the cell membrane via:
i) an interaction between the SLP-76 polypeptide and a membrane protein;
ii) a covalent bond between the SLP-76 polypeptide and a fatty acid in the membrane; and/or
iii) a binding between the SLP-76 polypeptide and a lipid polar head group in the membrane.

17. The composition of any one of claims 1 to 16, wherein the SLP-76 polypeptide comprises an amino acid sequence having at least 70% identity to SEQ ID NO: 1, 7, 14, 16, 18, 19, 40, 59 or 60.

18. The composition of any one of claims 1 to 17, wherein the isolated recombinant cell is an immune cell.

19. The composition of claim 18, wherein the isolated recombinant cell is a T cell, a tumor-infiltrating lymphocyte (TIL), a regulatory T cell (Treg), a natural killer (NK) cell, a macrophage, a monocyte, a gamma delta T cell, a stem cell, a natural killer T (NKT) cell, an induced pluripotent stem cell (iPSC)-derived NK cell, or an induced pluripotent stem cell (iPSC)-derived T cell.

20. The composition of any one of claims 1 to 19, wherein the isolated recombinant cell is capable of being activated by less than about 10, 100, 200, 300, 400, 500, 1000, 2000, 3000, 4000, 5000, 6000, 7000, 8000, 9000, or 10000, 20000, 30000, 40000, 50000, 60000, 70000, 80000, 90000, 100000, 200000, 300000, 400000, 500000, 600000, 700000, 800000, 900000, 1 million, 2 million, 3 million, 4 million, 5 million, 6 million, 7 million, 8 million, 9 million, or 10 million molecules of the target antigen.

21. The composition of any one of claims 1 to 20, wherein activation of the isolated recombinant cell in response to the target antigen enhances cell proliferation, differentiation, cytokine production and/or cytotoxicity.

22. An isolated polypeptide comprising a SLP-76 polypeptide comprising an amino acid sequence having at least 75% sequence identity to SEQ ID NO: 1, 7, 14, 16, 18, 19, 40, 59 or 60 and a transmembrane domain.

23. An isolated polynucleotide encoding an isolated membrane-bound SLP-76 polypeptide of claim 22.

24. An expression vector comprising an isolated polynucleotide of claim 23.

25. A host cell comprising an expression vector of claim 24.

26. A composition comprising
i) an isolated polynucleotide of claim 23 and an isolated polynucleotide encoding a T-cell receptor (TCR) polypeptide and/or a chimeric antigen receptor (CAR) polypeptide;
ii) an expression vector of claim 24 and an expression vector comprising an isolated polynucleotide encoding a T-cell receptor (TCR) polypeptide and/or a chimeric antigen receptor (CAR) polypeptide; and/or
iii) an expression vector comprising an isolated polynucleotide of claim 23 and isolated polynucleotide encoding a T-cell receptor (TCR) polypeptide and/or a chimeric antigen receptor (CAR) polypeptide.

27. A method of producing a recombinant cell, comprising introducing a composition comprising a polynucleotide encoding a SLP-76 polypeptide into a cell.

28. The method of claim 27, wherein the cell further comprises a TCR or CAR molecule.

29. A method of producing a recombinant cell, comprising introducing a composition comprising a polynucleotide encoding a SLP-76 polypeptide and a polynucleotide encoding a T-cell receptor (TCR) polypeptide and/or a chimeric antigen receptor (CAR) polypeptide into a cell.

30. A method of enhancing activation of a cell in response to a target antigen, comprising overexpressing a SLP-76 polypeptide in the cell.

31. The method of claim 30, wherein the cell further comprises a TCR or CAR molecule.

32. A method of enhancing activation of a cell in response to a target antigen, comprising overexpressing a SLP-76 polypeptide and a T-cell receptor (TCR) polypeptide and/or a chimeric antigen receptor (CAR) polypeptide in the cell.

33. A method of treating a disease or disorder in a subject in need of, comprising administering to the subject a pharmaceutically effective amount of a composition of any one of claims 1 to 21.

34. A method of treating a disease or disorder in a subject in need of, comprising
i) detecting the expression levels of a target antigen expressed by a host cell related to the disease or disorder in the subject, wherein the target antigen is recognizable by a T-cell receptor (TCR) polypeptide and/or a chimeric antigen receptor (CAR) polypeptide; and
ii) if the expression levels of the target antigen is lower than a control level, administering to the subject a pharmaceutically effective amount of T cells expressing the T-cell receptor (TCR) polypeptide and/or a chimeric antigen receptor (CAR) polypeptide and expressing a SLP-76 polypeptide.

35. The method of claim 34, further comprising a step, prior to step ii), of comparing the expression levels of the target antigen by the host cell to a control level.

36. The method of claim 34 or 35, further comprising a step, prior to step ii), of overexpressing the SLP-76 polypeptide in T cells expressing the T-cell receptor (TCR) polypeptide and/or a chimeric antigen receptor (CAR) polypeptide.

37. The method of any one of claims 34 to 36, wherein the SLP-76 polypeptide is membrane-bound.

38. The method of any one of claims 34 to 37, wherein the T cells expressing the T-cell receptor (TCR) polypeptide and/or a chimeric antigen receptor (CAR) polypeptide but not the SLP-76 polypeptide cannot treat or can only ineffectively or insufficiently treat the disease or disorder.

39. A method of treating a disease or disorder in a subject in need of, comprising
i) isolating at least one T cell from the subject, wherein the at least one T cell expresses a T-cell receptor (TCR) polypeptide and/or a chimeric antigen receptor (CAR) polypeptide, wherein the TCR polypeptide and/or the CAR polypeptide specifically recognizes a target antigen expressed by a host cell related to the disease or disorder in the subject;
ii) expressing a SLP-76 polypeptide in the isolated at least one T cell; and
iii) administering to the subject a pharmaceutically effective amount of the isolated T cell expressing the SLP-76 polypeptide.

40. The method of claim 39, wherein the levels of the target antigen expressed by the host cell is less than a control level.

41. The method of claim 39 or 40, further comprising a step, prior to step i), of comparing the expression levels of the target antigen by the host cell to a control level.

42. The method of any one of claims 39 to 41, wherein the SLP-76 polypeptide is membrane-bound.

43. The method of any one of claims 39 to 42, wherein the at least one T cell in the subject is a tumor infiltrating lymphocyte (TIL).

44. The method of any one of claims 39 to 43, wherein expressing the SLP-76 polypeptide enhances the activity of the at least one T cell to inhibit or kill the host cell.

45. The method of any one of claims 39 to 44, wherein the host cell is a cancer or tumor cell.
